(19) United States
(12) Patent Application Publication
(10) Pub. No.: US 2011/0082555 A1
(43) Pub. Date: Apr. 7, 2011

(54) Transforaminal Lumbar Interbody Fusion (TLIF) Implant, Surgical Procedure and Instruments for Insertion of Spinal Implant in a Spinal Disc Space

(76) Inventors: Erik O. Martz, Savage, MN (US); John Kuras, Red Bank, NJ (US); John Winterbottom, Howell, NJ (US); Craig J. Stratton, Forked River, NJ (US); David S. Marques, Morganville, NJ (US); Lawrene A. Shimp, Morganville, NJ (US)

(21) Appl. No.: 12/942,299

(22) Filed: Nov. 9, 2010

Related U.S. Application Data
(63) Continuation of application No. 10/912,667, filed on Aug. 5, 2004, now abandoned.
(60) Provisional application No. 60/495,646, filed on Aug. 15, 2003.

(51) Int. Cl. A61F 2/44

(52) U.S. Cl. 623/17.16

(57) ABSTRACT

Various instrumentation, implants and methodology are disclosed for implanting bone implants in the TLIF approach. The implants are preferably cortical bone of various shapes. The instruments include chisels, rasps, trials, inserters, spreaders, adjustors, curettes, rongeurs, and impactors. The instruments have straight and bent shafts. The implants may have recesses or notches in their sides for receipt of a mating insertion instrument. Some of the implants have a threaded hole for receiving a mating threaded stud of an implant insertion instrument. The implants may have saw tooth vertebral gripping surfaces which are lordotic or parallel, may be C-shaped, multi-faceted or annular.

This application is a Continuation of U.S. application Ser. No. 10/912,667, filed Aug. 5, 2004, which claims the benefit of provisional application No. 60/495,646 filed Aug. 15, 2003, entitled “Transforaminal Lumbar Interbody Fusion (TLIF) Implant, Surgical Procedure and Instruments for Insertion of Spinal Implant in a Spinal Disc Space”, the contents of all of which are hereby incorporated in their entirety by reference.

### CROSS REFERENCE TO RELATED ART

Of interest is commonly owned U.S. provisional application Ser. No. 60/495,646 filed Aug. 15, 2003 entitled Transforaminal Lumbar Interbody Fusion (TLIF) Implant, Surgical Procedure and Instruments for Insertion of Spinal Implant in a Spinal Disc Space, and U.S. patent application Ser. Nos. 60/340,734 filed Oct. 30, 2001 and 60/372,972 filed Apr. 16, 2002 entitled spinal Intervertebral Implant and insertion tools, corresponding PCT application serial no. PCT/US02/34466 and corresponding U.S. patent application Ser. No. 10/282,552 entitled Bone Implant and Insertion Tools, U.S. Patent application Ser. No. 60/425,941 filed Nov. 13, 2002 entitled Spinal Implant Insertion Adjustment Instrument and Implants for use therewith, now U.S. application Ser. No. 10/700,200 filed Nov. 3, 2003, in the name of Jo-Wen Lin and U.S. Pat. No. 6,277,149 entitled Ramp-Shaped Intervertebral implant to Boyle et al. which discloses a spinal implant and commonly owned US Published application no. 2002/0016633 to Lin entitled intervertebral spacer and implant insertion instrumentation which discloses an intervertebral spacer, all incorporated in their entirety by reference herein.

### FIELD OF THE INVENTION

This invention relates to spinal implants, instruments and method of preparing the spinal intervertebral disc space employing such instruments for insertion of implants into a intervertebral disc space.

### BACKGROUND OF THE INVENTION

Spinal implants, sometimes referred to as grafts, inserts or spacers, are in wide use and typically comprise non-bone physiologically compatible metal or other non-bone materials or bone. Reference is made to the aforementioned patent applications for description of bone implants. The method of preparing the site of the spine for spinal implant insertion involves a variety of tools and individual processes. The prior art is replete with different implants, implant insertion tools and procedures for insertion of spinal implants with such tools.

Patents which may reflect the state of the prior art include, for example, U.S. Pat. No. 6,096,038 to Michelson which discloses distraction tools for distraction of adjacent vertebrae, implants for insertion into the spine, drills for drilling the intervertebral site to prepare the site for implant insertion, other tools used for preparing the disc space by cutting bone, a driver extraction instrument for extracting an implant driver tool from the spinal disc space and generally discloses surgery for providing an integrated discectomy, fusion and interbody internal spinal fixation.

U.S. Pat. No. 6,174,311 to Branch discloses implants formed from donor bone for use in lumbar interbody fusion procedures and instruments for performing such procedures. Specific implants and instruments are disclosed for inserting the implants and for preparing the intervertebral space to receive the implants. Disclosed is a box chisel that has a hollow core that is somewhat rectangular.

Also disclosed is a plane scraper and a rotatable cutter. This latter cutter has multiple cutting arms defining a cavity therebetween for receiving cutting debris. Each arm has at least two cutting blades. The blades extend axially between the handle and the cutting end. A box chisel cutting edges are normal to the axial direction of the tool in a direction from the handle to the box cutter, whereas the rotating cutter cutting edges are parallel to the axial direction. In use, this rotating cutter tool cuts bone by rotation of the tool about its longitudinal axis.

U.S. Pat. No. 4,697,586 to Ganzale discloses a combined chisel-guide surgical instrument. The instrument is for performing osteotomy and other procedures on the human vertebra and comprises at least one longitudinally directed and movable chisel each including at least one front cutting edge for penetrating into the vertebra, a longitudinally directed guide including a front guide tip being locatable within intervertebral space for accommodating and directing the motion of the chisel cutting edges into the vertebra, a handle fixedly secured to rear extension of the guide for directing and placing the guide tip into the intervertebral space, a front impact block member connected to the rear extension of the chisel, an intermediate longitudinally directed cylindrical member connected to the rear end of the front impact block member, a rear impact cylindrical member fixedly connected to the rear end of the intermediate cylindrical member, and a longitudinally movable impact hammer accommodated by the intermediate cylindrical member.

U.S. Pat. No. 4,736,738 to Lipovsek et al. discloses an instrument kit and method for performing posterior lumbar interbody fusion. The kit includes first and second chisels and first and second shafts, a retaining ring with a set screw, an extraction hammer, a tamper and a hook.

U.S. Pat. No. 695,783 discloses a coping tool or chisel having a contour of molding to be cut and comprises a double chisel.

U.S. Pat. No. 740,937 discloses a chisel with a forward end with projecting spurs having rounded cutting edges. A forward end portion has a cutting edge.

U.S. Pat. No. 3,848,601 to Ma et al. discloses an interbody fusion apparatus including an intervertebral mortising chisel with an inner drill bit. The sides of the chisel have stops.

U.S. Pat. No. 5,722,977 to Wilhelmy discloses a quadrilateral osteotome (box chisel) for use with a guide spacer.

U.S. Pat. No. 6,224,607 to Michelson discloses an instrument set that includes an extended guard for providing protected access to the disc space, and the adjacent surfaces of the adjacent vertebral bodies, a guide insertable into the guard, and a bone removal device such as a drill insertable into the guide.

U.S. Pat. No. 6,436,101 to Hamada discloses a rasp for spine surgery.

U.S. Pat. No. 6,425,920 to Hamada discloses a spinal fusion implant for use in spine surgery.

US Published application no. 2003/0036764 to Hamada discloses spinal fusion implants, instrumentation and method. The instruments include a distractor, a rasp, a notcher, a chisel, a curette, a femoral ring bone implant, an implant holder and impact tool, a sizing tool, and a vertebral spreading device.

US Published application no. 2003/0130737 to McGahan discloses an anterior impacted bone graft and driver instruments.

US Published application no. 2003/0028249 to Bacelli et al. discloses an Intervertebral Implant with toothed faces.

US Published application no. 2003/0040798 to Michelson discloses lordotic interbody spinal fusion implants.

US Published application no. 2003/0060886 to Van Hoeck et al. discloses intervertebral spacers.

U.S. Design Pat. No. Des. 312,309 discloses a lumbar interbody graft driver.

US Published application no. 2003/0125739 discloses bio-active spinal implants and method of manufacture thereof.

US Published application no. 2003/0139815 to Grooms et al. discloses cortical bone-based composite implants.

US Published application no. 2002/0068941 to Hanson et al. discloses bone preparation instruments and methods.

U.S. Pat. No. 5,522,899 to Michelson discloses artificial spinal fusion implants and method for replacing spinal disc.

US Published application no. 2002/0077632 to Tsou discloses method and apparatus for spinal surgery. Disclosed are a spreader, a debrider, an obturator, a beveled cannula, and an intertebral implant.

US Published application no. 2002/0156530 to Lambrecht et al. discloses intervertebral diagnostic and manipulation device.

US Published applications no. 2001/0010001 and 0010002 to Michelson discloses instrumentation and methods for creating an intervetebral space for receiving an implant.

US Published application no. 2002/0019637 to Frey et al. discloses devices and techniques for a posterior lateral disc space approach. Disclosed are spreaders, a distractor, a reamer curved and straight, a rotary cutter, a push and a pull scraper, a straight chisel, a guided chisel, an implant sizing guide, an insertion guide, a curved implant inserter, an implant impaction tool, a guided implant inserter, implants bilaterally implanted in the disc space, an intradiscal rasp, an implant and instrument set for implanting the implant, and implants.

US Published application no. 2002/0138143 to Grooms et al. discloses cortical bone cervical Smith-Robinson fusion implant.

US Published application no. 2002/0068941 to Hanson et al. discloses bone preparation instruments and methods.

US Published application no. 2002/0072752 to Zucherman et al. discloses interspinous process implant sizer and distractor with a split head and size indicator and method.

US Published application no. 2002/0111679 to Zucherman et al. discloses apparatus and method for determining implant size.

US Published application no. 2002/0107523 to Naughton et al. discloses medical impacting device and system.

US Published application no. 2002/0165612 to Gerber et al. discloses intervertebral implant for transforaminal posterior lumbar interbody procedure and instrumentation.

US Published application no. 2002/0065560 and 2002/0065558 to Varga et al. disclose an intervertebral spacing implant system and a method of implanting an intervertebral spacer and U.S. Pat. No. 6,579,318 to Varga discloses an intervertebral spacer.

U.S. Pat. No. 6,500,206 to Bryan discloses instruments for inserting a spinal vertebral implant.

U.S. Pat. No. 6,261,296 to Aebi et al. discloses a spinal disc space distractor.

U.S. Pat. No. 6,511,509 to Ford et al. discloses a textured bone allograft and method of making and using same.

U.S. Pat. No. Des. 439,338 to Huttner discloses a curette tip.

The present inventors recognize a need for an improved TLIF implants, instrumentation and procedure for preparing and insertion of a spinal fusion implant into a spinal intervertebral disc space.

### SUMMARY OF THE INVENTION

According to an aspect of the present invention, a spinal bone implant is provided having a body made of bone. The body defines a longitudinal axis, and has opposing end walls transverse to the longitudinal axis, substantially parallel top and bottom surfaces, and opposing side walls in communication with the top and bottom surfaces and end walls. One of the side walls has a concave portion and the other side wall has a convex portion in opposing relation to the concave portion. The top and bottom surfaces are for bearing against respective adjacent vertebrae defining a disc space therebetween. Spaced parallel ridges are on the top and bottom surfaces transverse to the longitudinal axis. The body has a bore in one of the end surfaces and side walls in communication with a body outer peripheral surface extending inwardly towards a central region of the body.

In another aspect at least one of the top and bottom surfaces includes a plurality of spaced concentric ridges, and preferably, both the top and bottom surfaces of the body include a plurality of spaced concentric ridges.

In a further aspect, each of the plurality of spaced concentric ridges has two inclined side walls defining an angular relation to each other and converging at an edge. Each of the side walls of each of the ridges has substantially the same angular relation to each other to provide symmetrical ridging along the top and bottom surfaces.

In a further aspect, one of the side walls of the implant defines at least two notches in spaced relation to each other. The notches have internal side walls in angular relation to each other.

In a further aspect, the internal side walls of each of the notches are at an acute angle to each other.

In another aspect of the present invention a spinal bone implant comprises a body made of bone and has top and bottom surfaces defining top and bottom planes, respectively, and opposing side walls. The body defines a substantially central hole therethrough having a radius from a central longitudinal axis. The top and bottom surfaces are for bearing against respective adjacent vertebrae defining a disc space therebetween. Both top and bottom surfaces include a plurality of spaced linear ridges where the ridges on the top surface extend transverse to the ridges on the bottom surface.

In a further aspect, the ridges on the top and bottom surfaces are oriented orthogonal relative to each other.

In a further aspect, one of the side walls of the implant defines at least two notches in spaced relation to each other, the notches having two internal side walls inclined relative to each other.

In another aspect of the present invention, a spinal bone implant comprises

a body made of bone having an outer peripheral surface, top and bottom surfaces, the outer peripheral surface including a side wall. A cavity passes through the body in communication with the top and bottom surfaces. The top and bottom surfaces are for bearing against respective adjacent vertebrae defining a disc space therebetween. A bore is in the body in communication with an outer peripheral surface extending inwardly towards the cavity. The side wall defines at least two notches in spaced relation to each other where each notch has an internal wall transverse to the bore for receiving an implant insertion impact force.

In a further aspect, the peripheral surface has at least two notches in spaced relation to each other, and having internal side walls inclined to each other.

In a further aspect, the peripheral surface of the implant has an opening in communication with the notch, and the peripheral surface extends about the opening and has an arcuate section.

In a further aspect the perimeter has a planar section contiguous with the arcuate section.

In another aspect of the present invention, a chisel for preparing adjacent vertebrae for insertion of a spinal implant into the disc space defined by the vertebrae comprises a shaft having a longitudinal axis and distal and proximal ends, the shaft having a shaft head portion having a second longitudinal axis inclined relative to the shaft longitudinal axis and located at the shaft distal end. A handle is coupled to the shaft at the shaft proximal end. A cutting head is coupled to the shaft head portion extending distally from the shaft head portion, the cutting head terminating at a linear cutting edge extending inclined to the shaft axis and normal to the shaft head portion second longitudinal axis.

In a further aspect, the shaft portion second axis is at an angle of about 10 to 60 degrees to the shaft longitudinal axis.

In another aspect of the present invention, a curette for preparing adjacent vertebrae for insertion of a spinal implant into a disc space defined by the vertebrae comprises a shaft having a central longitudinal axis and distal and proximal ends. The curette includes a handle coupled to the shaft at a proximal end of the shaft. The curette head is coupled to the shaft at the shaft distal end, the curette head having a cutting surface that is oriented inclined to the longitudinal axis for scraping vertebral material, the cutting surface defining a perimeter of the curette head, the surface including spaced serrations.

The curette head is has a surface for scraping vertebral material inclined relative to the longitudinal axis. The surface defines a perimeter of the curette head, and the surface includes spaced serrations.

In a further aspect, the serrations include a plurality of coplanar linear teeth separated by notches.

In a further aspect, the shaft includes a distal shaft head portion terminating with the curette head. The distal shaft head portion is inclined to the longitudinal axis.

In a further aspect, the distal shaft head portion is at an angle of about 50.degree. to 60.degree. to the shaft the longitudinal axis.

In another aspect of the present invention, the distal shaft head portion of the curette is inclined to the longitudinal axis in plan and side elevation views.

In a further aspect, the shaft head portion is displaced from the longitudinal axis in one of plan and elevation views at an angle of about 50.degree. to 60.degree.

In a further aspect, the curette shaft head portion extends inclined to the longitudinal axis in elevation side view.

In a further aspect, the curette head is a loop curette.

In another aspect of the present invention, a lamina spreader for separating adjacent vertebrae for insertion and manipulation of a spinal implant in a disc space comprises an upper arm and a lower arm pivotally interconnected. The upper and lower arms each have a handle portion at a proximal end of the arms in spaced relation to each other and resiliently biased in opposite directions by a biasing device attached to the handle portions. The upper and lower arms each have a jaw at a distal arm end in spaced relation to each other forming spaced upper and lower jaws, each jaw terminating in a tip. A locking device is attached to the upper and lower handle portions for locking the handle portions in spaced relation against the bias.

In a further aspect, the upper and lower jaws are substantially parallel.

In a further aspect, the upper and lower jaw portions define a space therebetween between about 21 to 25 millimeters.

In another aspect of the present invention, a rasp for preparing vertebrae and a disc space between adjacent vertebrae during a spinal implant surgical procedure comprises a shaft having a longitudinal axis and distal and proximal ends. The rasp includes a handle coupled to the shaft proximal end, and a rasp head coupled to the shaft distal end. The rasp head has top and bottom surfaces and a side wall. At least one of the top and bottom surfaces includes a plurality of spaced teeth extending inclined to the top or bottom surface and toward the shaft proximal end.

In a further aspect, the teeth include a first side wall perpendicular to the top surface and a second side wall inclined to a first side wall, and the first and second side walls converge at a crest of each tooth.

In a further aspect, the crest is about 0.04 mm to the top or bottom surface.

In a further aspect, both the top and bottom surfaces include a plurality of symmetrical teeth symmetrically spaced.

In a further aspect, the shaft includes a distal shaft head portion terminating in the rasp head. The distal shaft head portion being inclined in one of side and plan views relative to the shaft longitudinal axis.

In a further aspect, the distal shaft head portion is inclined in the range of about 50.degree. to 60.degree. to the longitudinal axis.

In another aspect of the present invention, a tamp for manipulating and seating a spinal implant inserted into a disc space defined by two adjacent vertebrae comprises a shaft having a longitudinal axis and distal and proximal ends. A handle is coupled to the shaft at the shaft proximal end. The handle has an impact surface at the handle proximal end distal the shaft. A tamp head is coupled to the distal end of the shaft and has a planar distal end wall, a planar first side wall parallel to the shaft longitudinal axis, the distal end wall having an impact surface normal to the shaft longitudinal axis, and a second side wall inclined relative to the shaft longitudinal axis and relative to the head first side wall.

Preferably, the inclined side wall is at an angle of about 40.degree. to about 50.degree. to the shaft longitudinal axis.

In a further aspect, at least a portion of the impact surface includes knurls.

In a further aspect, the first side wall is normal to the impact surface and both include knurls.

In a further aspect, the knurls have inclined walls that are inclined to the surface at an angle of about 45.degree.

In a further aspect, the shaft has a distal inclined head portion terminating in the tamp head.

In a further aspect, the distal inclined shaft portion is inclined relative to the longitudinal axis in the range of about 40.degree. to 50.degree.

In another aspect of the present invention, a trial for measuring in a disc space defined by two adjacent vertebrae where the space is between adjacent vertebrae to size a spinal implant which comprises a shaft having a longitudinal axis and distal and proximal ends. A handle is coupled to the shaft proximal end. The shaft has a distal shaft portion terminating in the trial head, the distal shaft portion being inclined relative to the shaft longitudinal axis.

In a further aspect, the angle of inclination of the shaft portion is in the range of about 50.degree. and 60.degree.

In another aspect of the present invention, an implant insertion instrument comprises elongated first and second arms each having proximal and distal ends and a longitudinal axis and pivotally connected in a scissor-like arrangement. The first and second arms include opposing handle portions normally resiliently biased apart in spaced relation to each other at the arm proximal ends. The first and second arms terminate at opposing respective jaws at the arm distal ends. Displacement of the handle portions toward one another displaces the opposing jaws toward one another. Both the opposing jaws terminate with implant gripping tips, tips each have a planar end wall normal to the longitudinal axis and a side wall inclined to the longitudinal axis in opposing mirror image relation to each other. A locking device locks the handle portions in adjustable spaced relation against the bias.

In a further aspect, the angle between the tip side and end walls is about 30.degree.

In another aspect of the present invention, a method of inserting a spinal implant into an intervertebral disc space defined by adjacent vertebrae of a spine comprises:

a) distracting the disc space;

b) forming an opening in the perimeter of the disc space on a lateral or contralateral side of the disc space;

c) preparing the disc space on the lateral and contralateral sides of the space through the opening using at least one of a cup curette and a loop curette including a curette with a shaft head portion inclined to the longitudinal axis of the shaft;

d) inserting a rasp including a rasp with a shaft head portion inclined to the longitudinal axis of the shaft through the opening to further prepare the disc space first on one of and then on the other of the lateral and contralateral sides;

e) measuring the size of the lateral and contralateral sides with a trial including a trial with a shaft head portion inclined to the longitudinal axis of the trial shaft inserted through the opening;

f) repeating steps d-e until the disc space matches the size of an implant for insertion into that disc space; and then

h) inserting the at least one matched implant into the disc space.

In a further aspect, the method includes rotating the implant after it is inserted during or prior to displacing the implant.

In a further aspect, the method includes inserting a trial on a first of the sides to measure the size of the first of the sides, then displacing the trial from the first of the sides to a second of the sides, and inserting the trial in the second of the sides to measure the size of the second of the sides, and displacing the trial from the second of the sides.

In a further aspect, the method includes inserting an implant through an opening in a perimeter of the intervertebral disc space defined by adjacent vertebrae, and manipulating the implant in the disc space to a final implant position.

In a further aspect, the method includes orienting the implant inclined at an angle to the spinal anterior-posterior axis.

In a further aspect, the angle is such that a first side of the implant faces in either of two opposite directions transverse to the anterior-posterior axis.

In a further aspect, the angle is such that the implant is along a posterior side of the disc space.

In a further aspect, includes inserting a plurality of corresponding implants with corresponding sides facing the same direction.

In a further aspect, a plurality of corresponding implants have corresponding first sides facing each other.

In a further aspect, a plurality of corresponding implants have corresponding first sides facing in opposite directions.

In a further aspect, the method includes adjusting the position of the implant in the disc space with an L-shaped impact tool and impacting the implant into the final implant position.

In a further aspect, the method includes positioning the implant in the intervertebral disc space along an anterior wall defining the disc space and substantially perpendicular to the disc space anterior-posterior axis.

In a further aspect, the method includes positioning the implant in the intervertebral disc space diagonally across the disc space.

In a further aspect, the method includes positioning the implant in the intervertebral disc space along a posterior-anterior axis.

### BRIEF DESCRIPTION OF THE DRAWINGS

FIG. 1 is a plan view of a prior art posterior ramp insert;

FIG. 2 is a cross sectional side elevation view of the implant of FIG. 1 taken along line AA;

FIG. 3 is an end view of the implant of FIGS. 1 and 2 depicting a bore for receiving an implant insertion instrument prong;

FIG. 4 is a detail view of the ridges on the top and bottom surfaces of the implant of FIG. 2;

FIG. 5 is an end view of the implant opposite the end shown in FIG. 3;

FIG. 6 is a plan view of a C-shaped posterior implant according to an embodiment of the present invention;

FIG. 7 is a cross sectional side elevation view of the implant of FIG. 6 along line B-B;

FIG. 8 is an end view of the implant shown in FIGS. 6 and 7 depicting a bore for receiving an implant insertion instrument prong;

FIG. 9 is a detail view of the ridges shown in FIG. 7;

FIG. 10 is an end view of the opposite end of the implant end shown in FIG. 8;

FIG. 11 is a plan view of another embodiment of an implant according to an embodiment of the present invention having concentric ridges on opposite vertebrae engaging surfaces;

FIG. 12 is a cross-sectional side elevation view of the implant shown in FIG. 11 taken along line C-C;

FIG. 13 is an isometric view of the implant shown in FIGS. 11 and 12;

FIG. 14 is an end view of the implant shown in FIGS. 11-13 depicting a hole;

FIG. 14a is a detailed view of the ridges shown in FIG. 12;

FIG. 15 is a plan view of another embodiment of an implant according to an embodiment of the present invention where the array of parallel lines depict ridging on both the top and bottom surfaces, which ridges are oriented 90 degrees relative to each other;

FIG. 16 is a isometric view of the implant shown in FIG. 15 depicting the ridged surfaces;

FIG. 17 is a side elevation view of the implant shown in FIGS. 15 and 16;

FIG. 18 is an end view of the implant shown in FIGS. 15-17 depicting a bore for receiving an implant insertion instrument prong;

FIG. 19 is a detail view of the ridges shown in FIG. 16;

FIG. 20 is an isometric view of another embodiment of an implant according to an embodiment of the present invention;

FIG. 21 is a cross sectional view along line G-G in FIG. 24 depicting insertion tool receiving notches;

FIG. 22 is a cross sectional side elevation view along line F-F of the implant of FIG. 24 depicting ridges on top and bottom surfaces of the implant;

FIG. 23 is a detail view of the ridges shown in FIG. 22;

FIG. 24 is an end view of the implant shown in FIG. 20 depicting the notches;

FIG. 25 is a detail view of one of the notches shown in FIGS. 21 and 24;

FIG. 26 is a side elevation view of a prior art straight chisel depicting an inclined cutting edge four millimeters wide;

FIG. 27 is a plan view of the prior art chisel shown in FIG. 26 turned ninety degrees;

FIG. 28 is a side elevation view of a prior art straight chisel depicting an inclined cutting edge eight millimeters wide;

FIG. 29 is a plan view of the prior art chisel shown in FIG. 28 turned ninety degrees;

FIGS. 30 and 30a are respective side elevation and more detailed side elevation fragmented views of a chisel with an inclined shaft portion according to an embodiment of the present invention;

FIG. 31 is a plan view of the inclined chisel shown in FIG. 30 rotated ninety degrees;

FIG. 32 is a side elevation view of an inclined chisel according to an embodiment of the present invention which may be eight millimeters wide;

FIG. 33 is a plan view of the inclined chisel shown in FIG. 32 rotated ninety degrees;

FIG. 34 is a plan view of a surgical curette instrument according to an embodiment of the present invention;

FIG. 35 is a side elevation view of the instrument shown in FIG. 34 rotated ninety degrees;

FIG. 36 is a plan view of the shaft and curette of the instrument shown in FIGS. 34 and 35;

FIG. 37 is a side elevation view of the shaft shown in FIG. 36 rotated ninety degrees;

FIG. 38 is detail plan view of the curette shaft head portion of the instrument shown in FIGS. 34-37;

FIG. 39 is a detail plan view of the curette surface shown in FIG. 38 rotated ninety degrees;

FIG. 40 is a detail view of serrations shown in FIGS. 38 and 39;

FIG. 41 is a isometric view of a prior art handle for a surgical instrument;

FIG. 42 is side elevation view of the prior art handle shown in FIG. 41;

FIG. 43 is an end view of the prior art handle shown in FIGS. 41 and 42;

FIG. 44 is a plan view of a surgical instrument according to an embodiment of the present invention having an inclined shaft with a curette in a right orientation;

FIG. 45 is a side elevation view of the instrument shown in FIG. 44 depicting the upwardly inclined curette;

FIG. 46 is a plan view of the shaft of the instrument shown in FIGS. 44 and 45;

FIG. 47 is a side elevation view of the shaft shown in FIG. 46;

FIG. 48 is a detail view of the curette shaft head portion of the shaft shown in FIG. 47;

FIG. 49 is a detail view of the serrated teeth of the cup curette of the instrument shown in

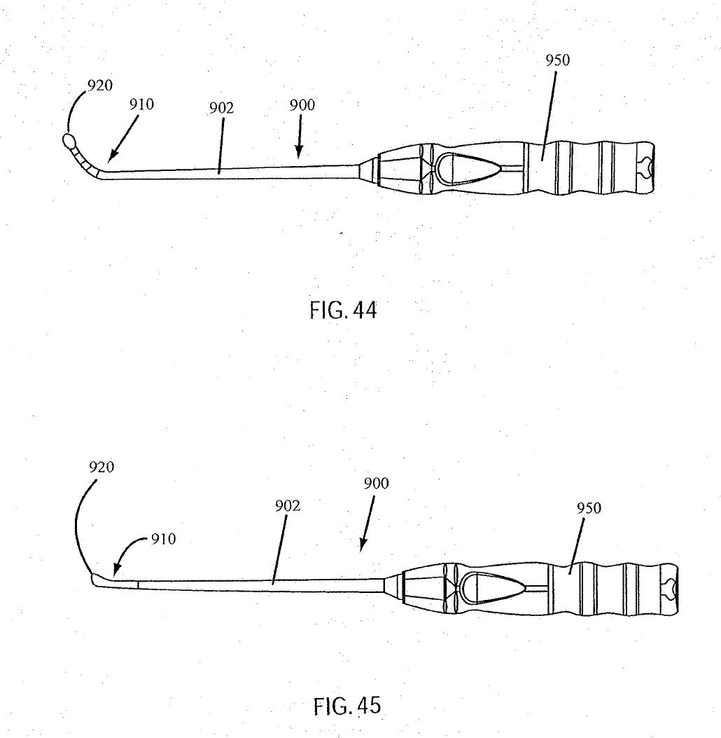

FIGS. 44-48;

FIG. 50 is a detail view of the inclined curette shaft head portion of the instrument shown in FIGS. 44-47;

FIG. 51 is a plan view of a surgical curette instrument according to an embodiment of the present invention having a left inclined shaft and curette;

FIGS. 51a and 52b are more detailed plan views of the curette tip portion of the instrument of FIG. 51 wherein in FIG. 51a the tip is shown inclined to the plane of the drawing sheet and in FIG. 52a the plane of the tip and drawing sheet are parallel;

FIG. 52 is a side elevation view of the instrument shown in FIG. 51 depicting the upwardly inclined curette;

FIG. 52a is a more detailed plan view of the tip portion of the instrument of FIG. 51;

FIG. 53 is a plan view of the shaft of the instrument shown in FIGS. 51 and 52;

FIG. 54 is a side elevation view of the shaft of the instrument shown in FIG. 53;

FIG. 55 is a detail view of the upwardly inclined head shown in FIG. 54;

FIG. 56 is a detail view of the serrated teeth of the cup curette shown in FIGS. 53-55;

FIG. 57 is a detail view of the inclined shaft head portion of the instrument shown in FIGS. 53 and 54;

FIGS. 58 and 59 are respective plan and side elevation views of a prior art surgical instrument having a straight loop curette;

FIG. 60 is a plan view of a prior art shaft of the surgical instrument shown in FIGS. 58 and 59;

FIG. 61 is a plan view of the prior art shaft shown in FIG. 60;

FIG. 62 is a detail view of the prior art curette shown in FIGS. 58-61;

FIG. 63 is a plan view of a surgical instrument according to an embodiment of the present invention having a left and downwardly inclined shaft head portion which includes a loop curette;

FIG. 64 is a side elevation view of the instrument shown in FIG. 63;

FIG. 65 is plan view of the shaft of the instrument shown in FIGS. 63 and 64;

FIG. 66 is a side elevation view of the shaft shown in FIG. 65;

FIG. 67 is a detail view of the shaft head portion including the loop curette shown in

FIGS. 63-66;

FIG. 68 is a plan view of a surgical instrument according to an embodiment of the present invention having a shaft head portion inclined to the right and downwardly;

FIG. 69 is a side elevation view of the instrument shown in FIG. 68;

FIG. 70 is a plan view of a shaft of the instrument shown in FIG. 68;

FIG. 71 is a side elevation view of the shaft shown in FIG. 70;

FIG. 72 is a detail view of the shaft head portion shown in FIGS. 68-72;

FIG. 73 is a side elevation view of a prior art surgical instrument having a shaft with a jaw actuation handle at one end and a pair of straight jaws at the opposite end;

FIG. 74 is a detail side elevation view of the prior art jaws shown in FIG. 73;

FIG. 75 is a detail plan view of a tip of one of the jaws shown in FIG. 74;

FIG. 76 is a side elevation view of a prior art surgical instrument having a shaft with a jaw actuation handle at one end and upwardly extending jaws at an opposite end;

FIG. 77 is a detailed side elevation view of the prior art jaws shown in FIG. 76;

FIG. 78 is a detail plan view of a tip of one of the jaws shown in FIG. 77;

FIGS. 79 and 80 are respective side elevation and plan views of a prior art lamina spreader having a pair of actuating handles opposite its jaws;

FIG. 81 is a detail view of the tips of the jaw portion shown in FIGS. 79 and 80;

FIGS. 82 and 83 are respective side elevation and plan views of a lamina spreader according to an embodiment of the present invention including offset jaws;

FIG. 84 is a detail view of the offset jaws of FIG. 82;

FIGS. 85 and 86 are respective side elevation and plan views of a surgical instrument according to an embodiment of the present invention including a shaft having a handle attached at one end and a rasp attached at an opposite end;

FIG. 86a is a side elevation view of a shaft of the surgical instrument shown in FIGS. 85 and 86;

FIG. 86b is a plan view of the shaft shown in FIG. 86a;

FIG. 86c is a detail isometric view of the rasp head shown in FIG. 86b;

FIG. 86d is a cross sectional view of the rasp taken along line A-A of FIG. 86b;

FIG. 86e is a cross sectional view of the rasp taken along line B-B of FIG. 86b;

FIGS. 87 and 88 are respective side elevation and plan views of a surgical instrument according to an embodiment of the present invention having a rasp portion at a distal end;

FIGS. 87a and 88a are more detailed views of the instrument tip portions of respective FIGS. 87 and 88;

FIGS. 89 and 90 are respective side elevation and plan views of a shaft of the surgical instrument shown in FIGS. 87-88;

FIG. 91 is a cross-sectional view of the rasp taken along line B-B in FIG. 90;

FIGS. 92 and 93 are detailed isometric and cross-sectional views of the rasp shown in FIG. 90;

FIGS. 94 and 95 are respective side elevation and plan views of a surgical tamp according to an embodiment of the present invention;

FIG. 96 is a detail perspective view of the rasp shown in FIGS. 94 and 95, depicting front and side impact surfaces;

FIG. 97 is a detail view of the side of the rasp shown in FIG. 96;

FIG. 98 is a plan view of the rasp shown in FIG. 96;

FIG. 99 is a plan view of a surgical tamp according to an embodiment of the present invention having an inclined tamp portion;

FIG. 100 is a side elevation view partially in section of the surgical tamp shown in FIG. 99;

FIG. 101 is a isometric view of the inclined tamp portion of the surgical tamp shown in FIG. 99 depicting the tamp head having a diamond patterned distal end;

FIG. 102 is a side elevation view of the tamp head shown in FIG. 101;

FIG. 103 is a plan view of the tamp head shown in FIG. 101;

FIG. 104 is an end view of the tamp head shown in FIGS. 101-103 depicting the diamond patterned front end of the tamp head;

FIG. 105 is a plan view of a trial (TLIF) having an inclined shaft head portion;

FIG. 106 is a detail view of the inclined shaft head portion of the trial (TLIF) shown in FIG. 105 accessing a disc space;

FIG. 107 is a partial cross sectional side elevation view along line D-D of FIG. 108 of the (TLIF) surgical trial instrument having a trial head on an inclined shaft portion;

FIG. 108 is a plan view of the trial (TLIF) surgical trial instrument shown in FIG. 107;

FIG. 109 is a detail view of the inclined head shown in FIG. 108;

FIG. 110 is a side elevation view of a prior art slap hammer instrument;

FIG. 111 is a side elevation view of a prior art surgical instrument for inserting a spinal implant;

FIG. 112 is a side elevation view of another TLIF surgical instrument according to an embodiment of the present invention holding a humeral implant;

FIG. 112a is a plan view of the TLIF surgical instrument shown in FIG. 112 depicting the implant in cross section along line E-E;

FIG. 112b is a partial detail view of the implant and partial view of the jaw members of the instrument as shown in FIG. 112a;

FIG. 112c is a detail cross sectional view of the implant shown in FIG. 112b depicting notches in the implant;

FIG. 112d is a isometric view of the TLIF surgical instrument and implant shown in

FIGS. 112 and 112a;

FIG. 113 is a plan view of a spinal implant for insertion in the disc space between adjacent vertebrae;

FIG. 114 is an end elevation view of the implant of FIG. 113;

FIG. 115 is a sectional side elevation view of the implant of FIG. 114 taken along line F-F;

FIGS. 116 and 117 are detailed views of the ridges on the top and bottom surfaces of the respective implants of FIGS. 115 and 114;

FIG. 118 is a side elevation view of the lumbar region of a spinal column having attached a pedicle screw system;

FIG. 119 is a rear view of the pedicle screw system shown in FIG. 118;

FIG. 120 is a rear view of the spinal column shown in FIGS. 118 and 119 without the pedicle screw system and with a facet joint removed;

FIG. 121 is a plan sectional view of a vertebral disc space having an opening;

FIG. 122 is a side elevation view of the spinal column shown in FIGS. 118 and 119 with an instrument representative of an offset lamina spreader instrument and another instrument representative of a pedicle screw distractor;

FIG. 123 is a side elevation view of the spinal column shown in FIG. 122 with an instrument representative of a scissor-type spreader;

FIG. 124 is a side elevation view of the spinal column shown in FIGS. 122 and 123 depicting the use of a chisel;

FIG. 125 is a plan sectional view of intervertebral disc space of FIG. 124 depicting the use of the chisel;

FIG. 126 is a plan sectional view of the vertebral disc space shown in FIG. 125 depicting the use of a representative instrument such as a curette;

FIG. 127 is a plan view of the vertebral disc space shown in FIG. 126 depicting the representative curette instrument with an inclined shaft portion including the curette and accessing the contralateral side of the disc space;

FIG. 128 is a plan view of the vertebral disc space shown in FIG. 127 depicting a rasp instrument;

FIG. 129 is a plan view of the vertebral disc space shown in FIG. 128 depicting the representative instrument having an inclined shaft portion and rasp head and accessing the lateral side of the disc space from a contralateral opening;

FIG. 130 is a plan sectional view of the vertebral disc space shown in FIG. 129 depicting an instrument representing a lordotic P-Ramp trial;

FIG. 131 is a plan sectional view of the vertebral disc space shown in FIG. 130 depicting an instrument representing inclined humeral spacer trials;

FIG. 132 is a plan sectional view of the vertebral disc space shown in FIG. 131 depicting the inclined instrument rotated 180 degrees and accessing the contralateral side of the disc space;

FIG. 133 is a plan sectional view of the vertebral disc space shown in FIG. 132 depicting an instrument inserting an implant into the disc space;

FIG. 134 is a plan sectional view of the vertebral disc space shown in FIG. 133 depicting the implant being inserted and rotated 180 degrees;

FIG. 135 is a plan sectional view of the vertebral disc space shown in FIG. 134 depicting an implant seated at the contralateral side of the disc space by a bone tamp instrument;

FIG. 136 is a plan view of the vertebral disc space shown in FIG. 135 depicting an implant seated at the anterior of the disc space by a bone tamp instrument;

FIG. 137 is a plan sectional view of the vertebral disc space shown in FIGS. 135 and 136 depicting inserts positioned contralaterally and medially within the disc space in facing spaced relationship and a bone tamp instrument;

FIG. 138 is a plan view of the vertebral disc space as shown in FIG. 137 depicting an implant positioned at the anterior of the disc space and another implant adjacent to the anterior implant and toward the posterior of the disc space in parallel spaced relationship;

FIG. 139 is a plan sectional view of the vertebral disc space as shown in FIG. 138 depicting an implant facing toward the lateral side of the disc space and another implant toward the medial side of the disc space where the implants are rotated 180 degrees from the orientation of the implants shown in FIG. 137;

FIG. 140 is a plan sectional view of the vertebral disc space as shown in FIGS. 131-139 depicting an inserter instrument inserting an implant into the disc space;

FIG. 141 is a plan sectional view of the vertebral disc space as shown in FIG. 140 depicting an implant positioned at the anterior side of the disc space with a bone tamp in the disc space;

FIG. 142 is a plan sectional view of the vertebral disc space as shown in FIG. 141 depicting an implant positioned diagonally along an anterior-medial plane;

FIG. 143 is a plan sectional view of the vertebral disc space as shown in FIG. 142 depicting an implant positioned along an anterior-posterior plane;

FIG. 144 is a side elevation partially in section view of the lumbar region of the spinal column shown in FIGS. 122-124 depicting the pedicle screw system locked in place;

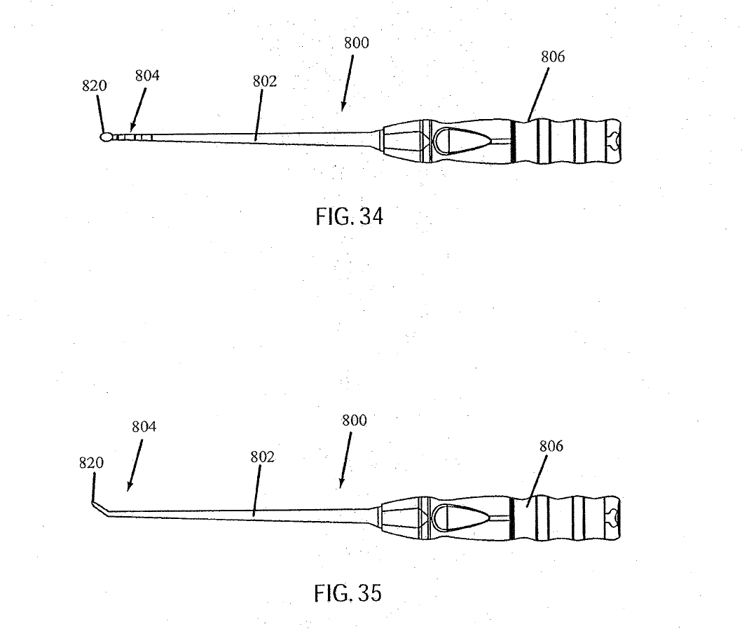

FIGS. 145 and 146 are respective plan and side elevation views of an alternative shaft and curette without serrations for use with the instrument shown in FIGS. 34-37;

FIG. 146a is a detail plan view of the shaft head portion of the shaft shown in FIG. 145;

FIG. 147 is a plan view of an alternative shaft and curette without serrations and inclined to the right for the instrument shown in FIGS. 44 and 45;

FIG. 148 is a side elevation view of the shaft and curette shown in FIG. 147;

FIG. 149 is a detail view of the curette head of the shaft shown in FIG. 148;

FIG. 149a is a detail view of the curette head and shaft portion shown in FIG. 147;

FIG. 150 is a plan view of an alternative shaft and loop curette similar to the shaft shown in FIGS. 65 and 66;

FIG. 151 is a side elevation view of the shaft shown in FIG. 150;

FIGS. 152 and 153 are respective side elevation and plan views of an alternative rasp shaft similar to the rasp shaft shown in FIGS. 89 and 90;

FIG. 154 is a side elevation view of an alternative trial having a tapered tip portion;

FIG. 155 is a detail side elevation view of the trial head of FIG. 154;

FIG. 156 is a plan view of the trial instrument of FIG. 154; and

FIG. 157 is a detail view of the inclined portion of the trial shown in FIG. 156.

### DETAILED DESCRIPTION OF THE PREFERRED EMBODIMENT(S)

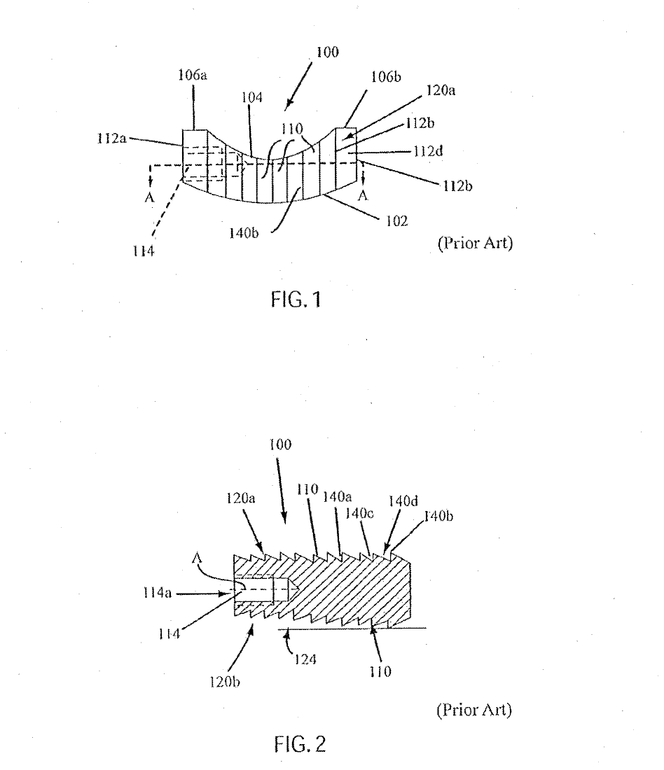

In FIGS. 1-5, a prior art posterior ramp implant 100 has a convex side wall 102 opposite a concave side wall 104. The term implant as used herein is sometimes referred to in this art as an insert, a ramp, a spacer or a graft, the term ramp referring to an implant that is ramp shaped, i.e., is elongated and has inclined vertebral gripping surfaces that define lordotic angles such as implant 100. Two coplanar walls 106a and 106b are in spaced relation along the same plane with the concave side wall 104 therebetween. The implant has two opposite end walls 112a and 112b. The implant 100 further has top and bottom surfaces 120a and 120b, respectively, as shown in FIG. 2. The top and bottom surfaces 120a, 120b include teeth 110 extending the length of the top and bottom surfaces 120a and 120b. The teeth 110 include a wall 140a perpendicular to the top and bottom surfaces 120a, 120b and longitudinal axis A, and a wall 140c inclined to the longitudinal axis A. The walls 140a and 140c converge at a linear edge 140b. A space 140d is defined between the inclined surface 140c and an adjacent perpendicular wall 140a. The teeth 110 on top surface 120a are representative of the teeth on the bottom surface 120b.

The implant 100 further includes a bore 114 having an opening 14a in side wall 112a. The opening 114a and bore 114 are configured to receive a mating prong of an implant insertion instrument (not shown) for positioning the implant into the spinal disc space between adjacent vertebrae.

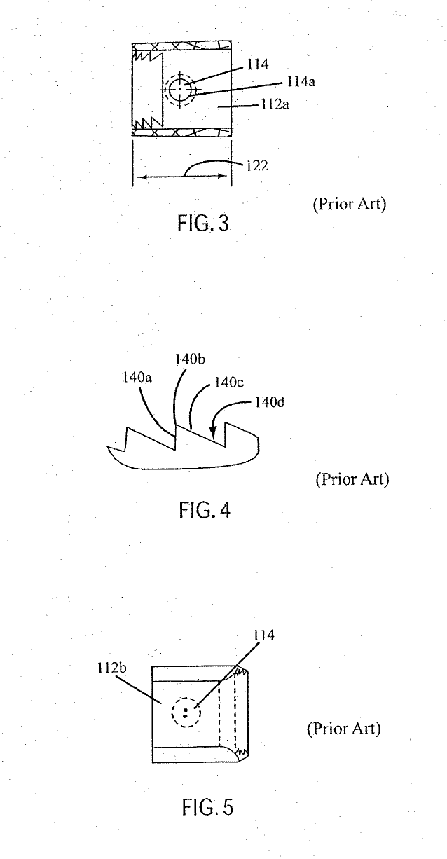

The posterior ramp implant 100 may have a width 122 of about 8.5 mm as shown in FIG. 3. The posterior ramp implant 100 is shaped to correct lordosis by having a lordotic angle 124 of about 2.5.degree.+−.0.3.degre-e., FIG. 2. The angle restores and maintains sagittal balance for proper loading. The angle 124 is representative of an angle between the plane of the top edges of the teeth 110 and a horizontal plane through the longitudinal axis A such that the top and bottom surfaces 120a and 120b are inclined relative to each other and to axis A.

The implant 100 is placed between the vertebrae so that the curved portion shown in FIG. 1 may be faces the spinal column, but may have other orientations as well such as facing one another wherein two implants are in the disc space in opposing relationship, for example. The ridged surfaces are shown in FIG. 2, face the upper and lower vertebrae after the implant is inserted, tend to dig into the adjacent vertebrae to retain the implant after insertion in the disc space.

Other posterior implants may have lordotic angles of about 5.degree., and an anterior implant may have 8.degree. lordotic angle.

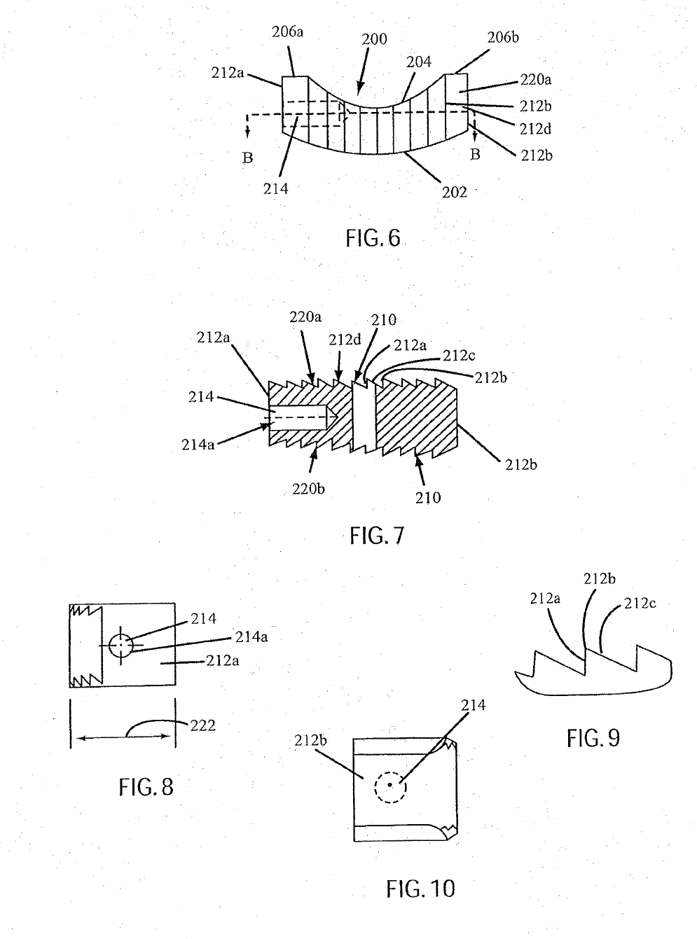

An embodiment of a non-lordotic posterior block implant 200 according to an embodiment of the present invention is preferably made of cortical bone, but may be other bone, shown in FIGS. 6-10. The implant 200 has a convex side wall 202 opposite a concave side wall 204. Two walls 206a and 206b have coplanar surfaces and are in spaced relation to each other with the concave side wall 204 therebetween. Two end walls 212a and 212b oppose each other. The implant 200 has top and bottom surfaces 220a and 220b, respectively. The top and bottom surfaces 220a, 220b include parallel linear teeth 210 in an array along the top and bottom surfaces 220a and 220b. The teeth 210 on top surface 220a are representative of the teeth on the bottom surface 220b. The teeth 210 include a wall 212a perpendicular to the top and bottom surfaces 220a, 220b and to the longitudinal axis of the implant corresponding to axis A FIG. 2. The wall 212a terminates at edge 212b contiguous with inclined surface 212c inclined relative to the longitudinal axis. A space 212d is defined between the inclined surface 212c and an adjacent perpendicular wall 212a.

The implant 200 includes a bore 214 having an opening 214a in side wall 212a. The opening 214a and bore 214 are configured to receive a mating prong of an implant insertion instrument (not shown) for positioning the implant into the spinal disc space between adjacent vertebrae.

The posterior ramp implant 200 may have a width 222 of 8.5 mm as shown in FIG. 8. The posterior ramp implant 200 is non-lordotic, i.e., the sides 220a and 220b are parallel, as shown in FIG. 7. The implant 200 is placed between the vertebrae so that the concave wall 204 shown in FIG. 6 faces the spinal column in one embodiment. In the alternative, it may be in other orientations as well and may also be located in the anterior region. The toothed surfaces 220a and 220b, FIG. 7, face the upper and lower vertebrae when the implant is inserted.

When the implant is inserted between adjacent vertebrae, top and bottom surfaces of adjacent vertebrae engage the implant toothed bottom and top surfaces 220a and 220b, respectively.

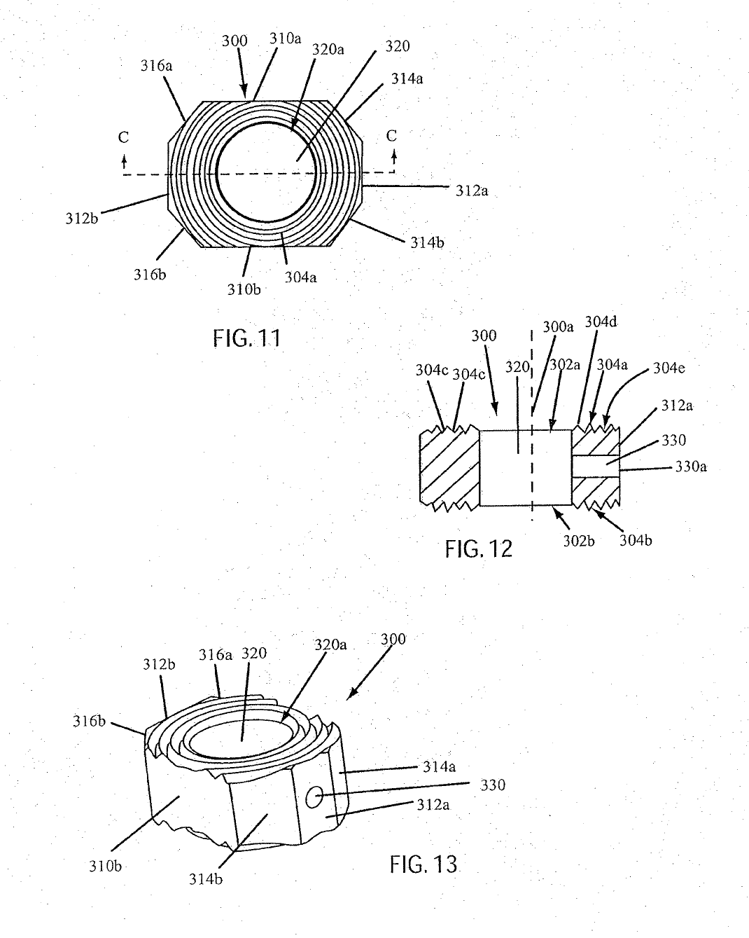

Another embodiment of an implant 300 according to an embodiment of the present invention is shown in FIGS. 11-14. The implant 300 is preferably cortical bone, but may be other bone, and is a transforaminal lumbar interbody fusion (hereinafter TLIF) implant. The implant has preferably parallel top and bottom surfaces 302a and 302b, FIG. 12. The top and bottom surfaces 302a, 302b, respectively, are farmed with concentric circular ridges 304a, 304b, respectively. The ridges on the top surface 302a, FIG. 11, is representative of circular ridges on the bottom surface 302b.

The circular ridges 304a are saw tooth in shape and extend radially equally spaced from each other from a central longitudinal axis 300a of the implant, FIG. 12. The ridges 304a each have two side walls 304c inclined relative to a plane normal to the axis 300a and whose inclination extends radially outwardly and converging at an upper crest edge 304d. Each pair of facing inclined side walls 304c of the corresponding ridge 304a defines a “V” shaped root or trough 304e therebetween. The circular ridges 304a resist motion in all directions of the implant 300 after it is inserted between adjacent vertebrae.

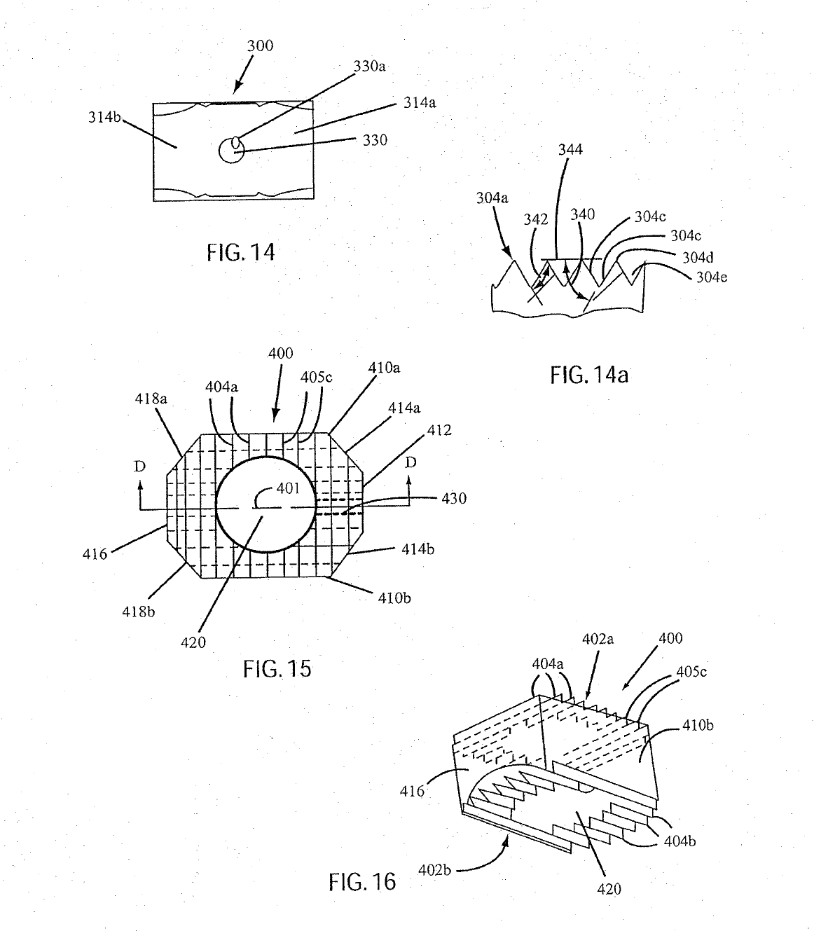

The ridges 304a are shown in greater detail in FIG. 14a. The inclined side walls 304c are preferably 60.degree. from a plane 344 defined by with the tooth crests 304d of the ridges 304a, as shown by arrows 340 and 342 in FIG. 14a.

In FIGS. 11 and 13, contiguous side walls circumscribe the perimeter of the implant 300 and include opposing planar side walls 310a and 310b, and opposing planar end walls 312a and 312b. Inclined intermediate walls 314a and 314b are on opposite sides of the wall 312a, and inclined intermediate walls 316a and 316b are on opposite sides of the wall 312b.

A circular cylindrical cavity 320 is centrally positioned in the implant in communication with the top and bottom ridged surfaces.

The wall 312a includes an a bore 330 extending into the implant as shown in FIG. 12. The bore 330 is configured to mate with a prong on an implant insertion tool (not shown).

Humeral spacer implant 400 according to a further embodiment, FIGS. 15-19, is for TLIF and is preferably cortical bone, but may be other bone, and has a shape similar to the implant 300, FIG. 13. It is preferably made from the transverse slice of a long bone. The implant 400 has respective top and bottom surfaces 402a and 402b which have parallel arrays of saw tooth shaped ridges 404a and 404b, respectively. The arrays of ridges 404a and 404b on the respective top and bottom surfaces are oriented 90 degrees from each other, FIGS. 15-16. In an alternative embodiment, the ridges may be symmetrical. The 90.degree. orientation of the ridges results in the top surface resisting motion in a first direction in a first plane, e.g., left to right in FIG. 15, and the bottom surface resisting motion in a second direction rotated 90 degrees from the first direction in a second plane parallel to the first plane, e.g., top to bottom of the drawing figure.

The ridges 404a on the top surface 402a are representative of the ridges 404b on the bottom surface 402b in regard to their geometry. The ridge 404a includes a first side wall 405a perpendicular to the plane of the top surface 402a and a second inclined side wall 405b with inclined respect to the plane of the top surface. The walls 405a and 405b intersect to form edge at crest 405c.

The implant 400 has contiguous side and end walls 410a, 414a, 412, 414b, 410b, 418b, 416, and 418a, defining the side-end wall perimeter of the implant. Side walls 410a and 410b are opposite one another, and end walls 412 and 416 are opposite one another and perpendicular to the plane of the walls 410a and 410b. Two intermediate walls 418a and 418b inclined to the side and end walls are on opposite edges of wall 416, and two intermediate walls 414a and 414b inclined to the side and end walls are on opposite edges of wall 412. The implant further includes a substantially central through cavity 420, which may be a machined medullary canal of a long bone, in communication with the top surface 402a and bottom surface 402b. The implant further includes a bore 430 extending into the implant 400 at wall 412 along the longitudinal implant axis 401. The bore 430 is configured to receive a mating prong on an implant insertion instrument (not shown) for inserting the implant into the disc space between adjacent vertebrae.

A ridge 404b is shown in greater detail in FIG. 19. Each ridge 404b includes the inclined wall 405b, perpendicular wall 405a and the crest edge 405c. Angle 440 is about 60.degree. between the perpendicular wall 405a and the inclined wall 405b.

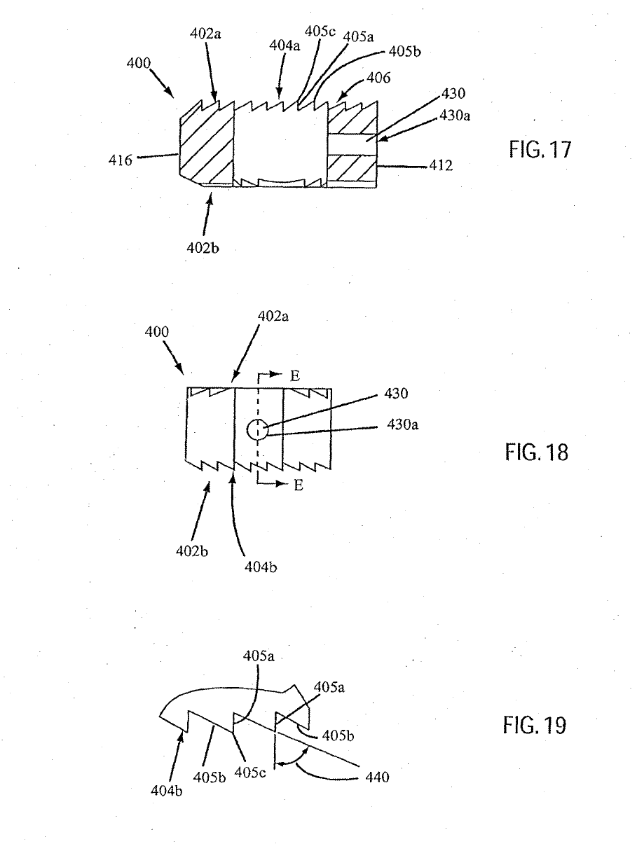

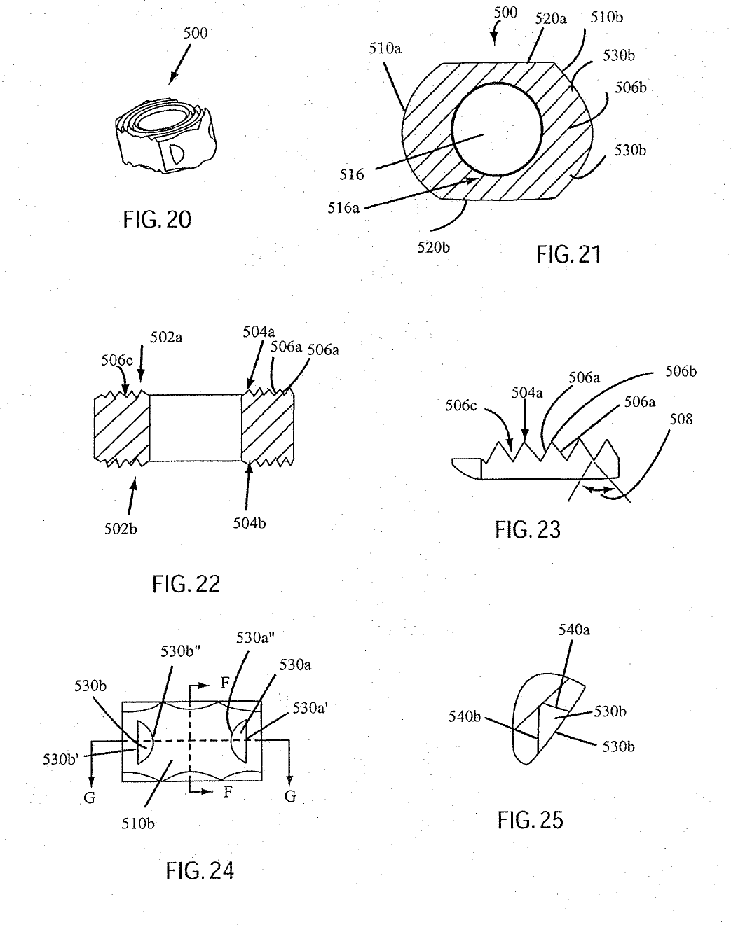

A bone implant 500 according to an embodiment of the present invention is shown in FIGS. 20-25 and also is preferably cortical bone made from a transverse slice of the long bone of an animal, preferably a human bone. The implant 500 has parallel top and bottom surfaces 502a and 502b. The top and bottom surfaces 502a, 502b include preferably circular concentric saw tooth shaped ridges 504a and 504b, respectively, as shown in FIGS. 20 and 22. The circular ridges 504a, 504b, include opposing inclined walls 506a extending outwardly and intersecting at a crest edge 506b, as shown in FIGS. 21 and 23. Adjacent inclined walls 506a define a gap 506c therebetween. The ridges 504a, 504b are preferably circular as shown in FIG. 20, however, the ridges on the upper and lower surfaces may also be linear or offset by 90.degree. relative to each other as in the implant embodiment 400 shown in FIGS. 16 and 17. The implant includes contiguous side walls consisting of opposing planar side walls 520a and 520b, and opposing convex side walls 510a and 510b. A substantially central cavity 516 passes through the implant in communication with the top surface 502a and bottom surface 502b and may be formed from the medullary canal of a bone.

The ridges 504a on the top surface 502a, FIG. 23, are representative of the ridges 504b on the bottom surface 502b. The ridges 504a include the inclined side walls 506a, crest edge 506, and root gap 506c formed between adjacent tooth walls 506a. The angle 508 between adjacent ridges is preferably about 60.degree.

The implant also includes oppositely disposed notches 530a and 530b in convex side wall 510b, FIGS. 21 and 24. The notch 530b has a planar wall 530b′ contiguous with an arcuate wall 530b″. Similarly, the notch 530a has a planar wall 530a′ and is contiguous with an arcuate wall 530a″. The two notches 530a and 530b are positioned in the convex side wall 510b. The notches 530a, 530b are in mirror image spaced relation to one another in the convex side wall 510b. The tips of jaws of a surgical instrument can be positioned in the notches to grasp the implant. The notch 530b is shown in greater detail in FIG. 25. The notch 530b has a vertical planar wall 540b angularly abutting with a planar wall 540a. The angle between the vertical planar wall 540b and planar wall 540a is preferably acute, i.e., less than 90 degrees. The angular relation of the walls 540a and 540b provides more positive gripping by the jaw tips of an instrument because the inclined wall 540a provides greater resistive forces to an exiting jaw tip in contact with the wall 540a. See copending PCT application serial no. PCT/US02/34466 published as WO 03/037228 and corresponding U.S. application Ser. No. 10/282,552 filed Oct. 29, 2002 all incorporated by reference herein.

A surgical instrument such as the TLIF humeral inserter shown in FIGS. 112-112b can be used to grasp the implant 500 and position it between adjacent vertebrae. The tips 2430a and 2430b of the TLIF humeral inserter fit into the notches 530a and 530b as shown in FIG. 112b, and are described in greater detail below.

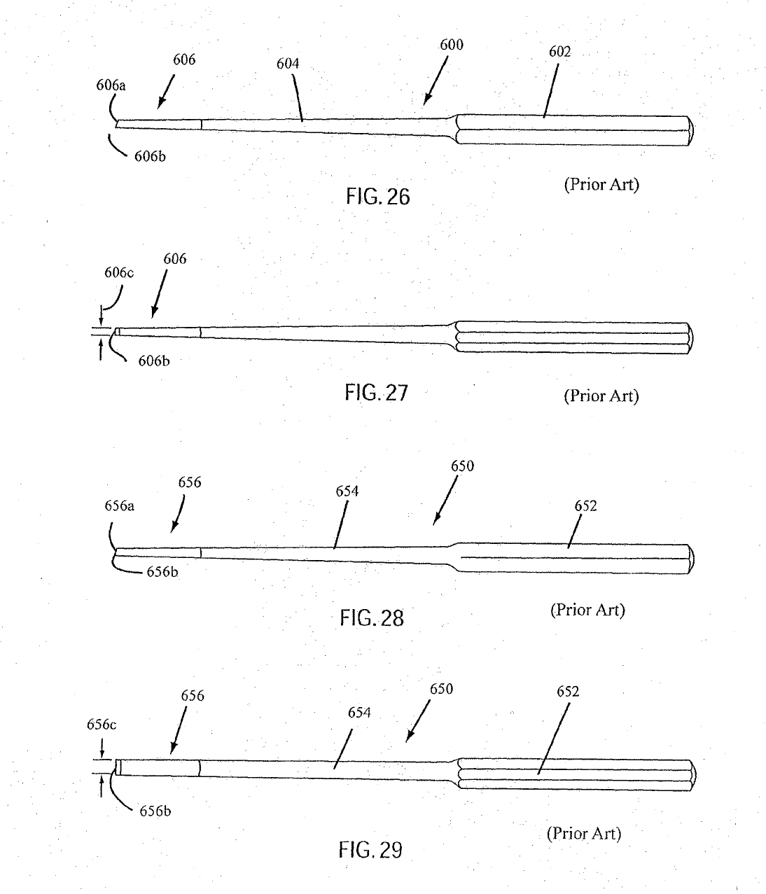

Prior art straight chisels are shown in FIGS. 26-29. The chisel 600 shown in FIGS. 26 and 27 includes a handle 602, shaft 604 and head 606 which are contiguous. The distal tip of the head 606 has an inclined surface 606a relative to the shaft longitudinal axis terminating in an edge 606b. The transverse width 606c of the edge 606b is preferably about 4 mm.

Another prior art straight chisel 650 is shown in FIGS. 28 and 29. The chisel 650, similar to chisel 600, also has a handle 652, shaft 654 and head 656 which are contiguous. The distal tip of the cutting head 656 has an inclined surface 656a relative to the shaft axis terminating in a cutting edge 656b. The width 656c of the edge 656b is preferably about s8 mm.

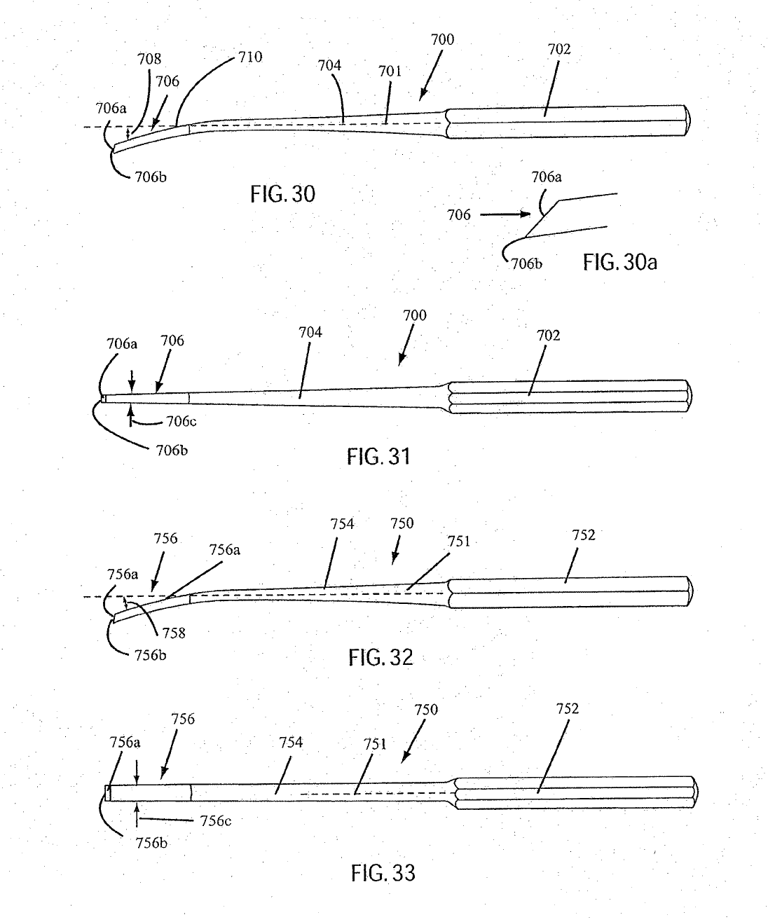

An chisel instrument 700 with an inclined head portion relative to the shaft longitudinal axis according to an embodiment of the present invention is shown in FIGS. 30, 30a and 31. The chisel 700 includes a generally frusto-conical tapered handle 702 with flat handle surfaces for ease of gripping. A head 706 is formed in one end of the shaft, the shaft being contiguous with the handle 702. The head 706 has an inclined distal surface 706a relative to the shaft axis terminating at cutting edge 706b for cutting bone and cartilage in the spinal vertebrae area. The width 706c of the edge 706b is preferably about 4 mm.

The head 706 shaft portion in an orientation as used by a surgeon is inclined downwardly from a longitudinal center axis 701 of the shaft 704. The inclination angle 708 is between the center axis 701 and an outer surface 710 of the shaft head portion. Preferably the angle 708 is about 150 with a tolerance of .+−0.5.degree. The inclined shaft portion 706 enables a surgeon to access areas of a vertebral cavity with greater ease. A surgeon determines the use of the chisel instrument 700 and predetermined width such as the 4 mm or other size cutting edge in a particular procedure.

A chisel instrument 750 according to a further embodiment of the present invention is shown in FIGS. 32 and 33, which is similar to the instrument 700 shown in FIGS. 30 and 31, however, the chisel instrument 750 has a wider cutting edge. The chisel 750 includes a handle 752 similar to the handle 702, and a shaft 754 which has an inclined portion 754a relative to the handle 752 longitudinal axis and extends toward a distal end. The shaft 754 has an inclined shaft head portion 756 which positions the chisel cutting head 756a at the desired angle from a center axis 751, FIG. 32. The handle 752, shaft 754 and head portion 756 are contiguous. The head 756a has an inclined distal cutting edge side surface terminating at a cutting edge 756b. The width 756c of the edge 756b is preferably about 8 mm, as shown in FIG. 33. The shaft head portion 756 when in use by a surgeon is inclined vertically downwardly from the center axis 751 of the shaft 754. The angle of inclination 758 is between the center axis 751 and a top surface of the head 756a and the angle 758 is preferably about 15.degree. with a tolerance of .+−0.5.degree. A surgeon may prefer to use the instrument 750 with the inclined shaft head portion 756 and the 8 mm cutting edge to access areas of a vertebral cavity.

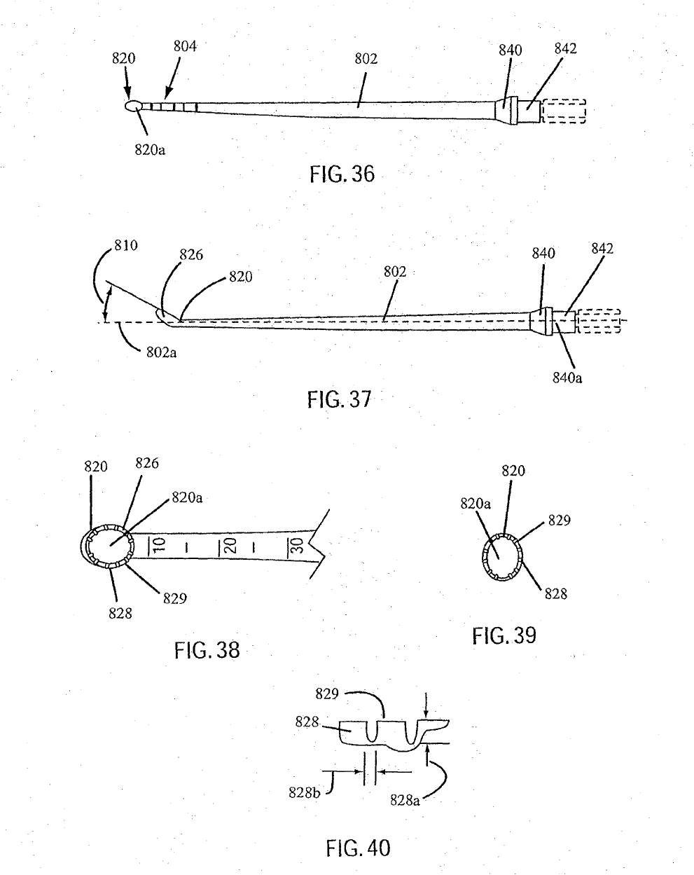

A curette surgical instrument 800 according to a further embodiment of the present invention is shown in FIGS. 34 and 35. The instrument 800 includes a frusto-conical tapered shaft 802 having a curette head portion 804 at a distal end of the shaft 802 terminating with a curette head 820. The curette head 820 has a serrated edges in the form of an oval shaped ring-like scraping or cutting edge 826, FIG. 38. The shaft 802 is connected to a prior art cylindrical handle 806 at a proximal end of the shaft 802. A more detailed depiction of the shaft 802 is shown in FIGS. 36-38. The shaft 802 has a frusto-conical portion 840 connected to a collar 842. The collar 842 connects to a receiving hole in the handle 806 such that an end wall of the handle abuts an end wall 840a of the frusto-conical portion 840. The shaft portion 804 is inclined upwardly relative to the handle longitudinal axis in the use orientation by an angle 810 preferably about 30.degree.+−0.5.degree. between a central axis 802a of the shaft 802 and the surface 826 of the curette head 820. The curette head 820 is substantially oval shaped and the head 820 and cutting surface 826 is shown in more detail in FIGS. 38-40. The cutting surface 826 of the head 820 defines a perimeter about a hollow through core region 820a. The cutting surface 826 includes serrations having grooves 828 separated by planar surfaces 829 along the curette head surface 826. The surface 826 enhances the ability to scrape and remove material from a vertebral area. In the preferred embodiment shown in FIGS. 38-40 of the serrated curette head 820, the depth 828a of the groove 828 is preferably about 1 mm, and the width 828b of the groove 828 is preferably 0.5 mm.

Alternatively, the curette instrument 800 may have an elongated tapered shaft 3350 shown in FIGS. 145, 146. The shaft 3350 has a curette shaft head portion 3354 terminating with a curette head 3358 at a distal end of the shaft 3350. The curette head 3358 is oval shaped and has a substantially smooth scraping surface 3360. The scraping surface 3360 is along a perimeter of the curette head 3358 defining through opening 3358a. The shaft head portion 3354 may include depth markings as shown in FIG. 146a. Each marking 3370a-3370e is from the end of the curette head 3358 as indicated by corresponding dimension lines 3380, FIG. 146a. Marking 3370a indicates a depth of 10 mm, marking 3370b is for 15 mm, marking 3370c is for 20 mm, marking 3370d is for 25 mm, and marking 3370 is for 30 mm. The markings 3370a-3370e indicate to a surgeon the depth of the curette in a vertebral disc space.

The shaft 3350 includes a frusto-conical portion 3362 connected to a cylindrical collar 3362a similar to the shaft 802 shown in FIGS. 36 and 37. The collar 3362a connects to a receiving bore in the handle 806 such that an end wall of the handle abuts an end wall 3362b of the frusto-conical portion 3362. The head 3358 is inclined upwardly relative to the shaft portion 3354 by an angle 3356a preferably about 30.degree.+−0.5.degree. between a central axis 3356 of the shaft 3350 and the cutting surface 3360 of the curette head 3358.

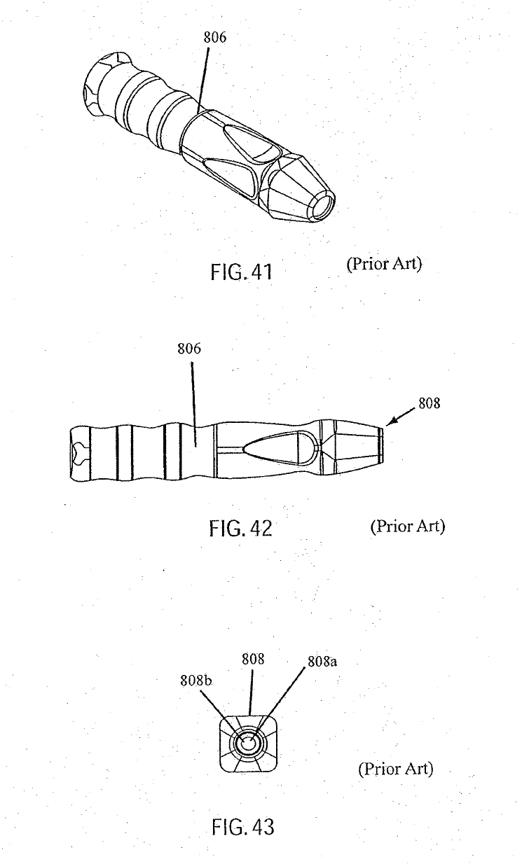

The prior art handle 806 shown in FIGS. 34 and 35 is shown in greater detail in FIGS. 41-43. The handle 806 has a receiving end 808 for mating with a shaft, such as shaft 3350 shown in FIG. 145 or shaft 802 shown in FIG. 36. The handle defines a bore 808a with an opening 808b at the receiving end 808. The bore 808a is adapted to receive the cylindrical collar of a shaft. The handle may have a silicone outer material and an aluminum core.

In FIGS. 44 and 45, serrated cup curette instrument 900 includes a shaft 902 having a shaft head portion 910 terminating with a curette head 920 at a distal end. The shaft 902 is also connected to a prior art handle 950 which is essentially the same as handle 850, and is essentially the same as prior art handle 806 shown in FIG. 41.

The shaft head portion 910 of the shaft 902 is inclined upwardly at an angle 975 between a central longitudinal axis 905 through the shaft 902 and a scraping or cutting surface 922, as shown in FIGS. 47 and 48. The angle 975 is preferably about 30.degree.+−0.5.degree. The shaft head portion 910 is also inclined toward the right at an angle 925 between the central longitudinal axis 905 of the shaft 902 and an axis 905a passing through the center of the curette head 920. The angle 925 is preferably about 55.degree.+−0.5.degree. The angle of the shaft head portion of the curette allows the surgeon to access the contralateral side (the side opposite an opening to the disc space) of the disc space through a single opening to the disc space at one side of the disc space.

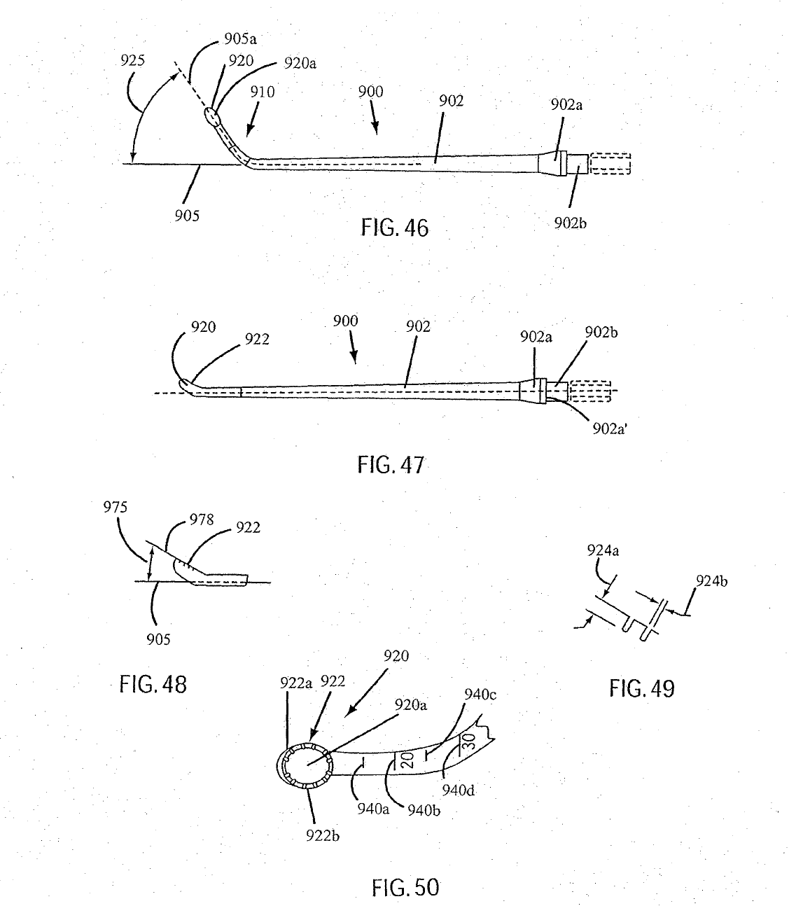

The proximal end of the shaft 902 shown in more detail in FIGS. 46 and 47 is similar to the shaft 802 shown in FIGS. 36 and 37. The shaft 902 includes a frusto-conical portion 902a connected to a cylindrical collar 902b. The collar 902b connects to a receiving hole in the handle 950 such that an end wall of the handle abuts an end wall 902a′ of the frusto-conical portion 902a.

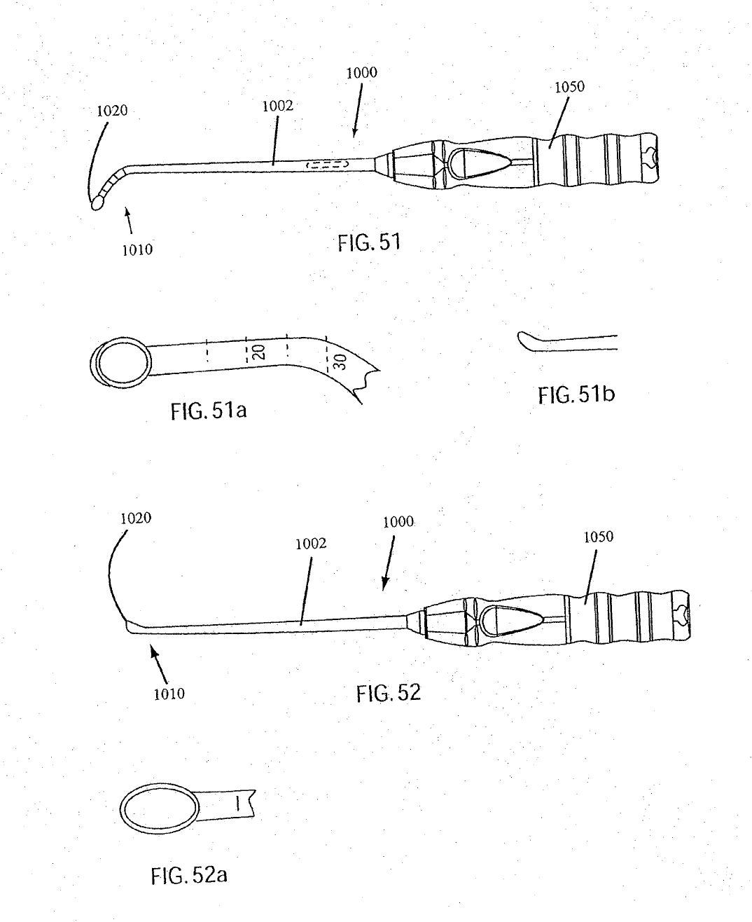

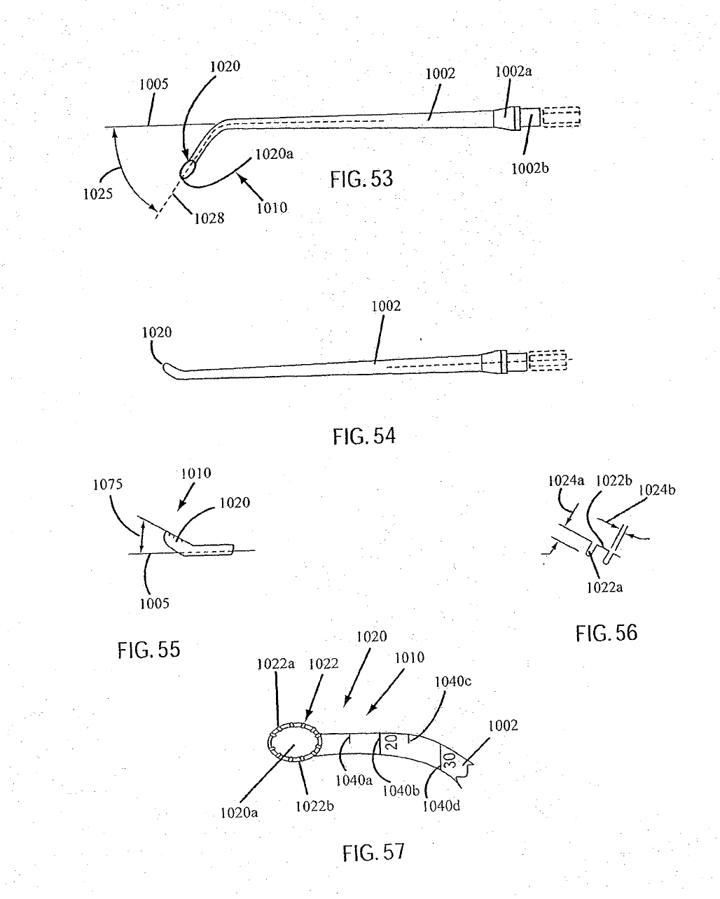

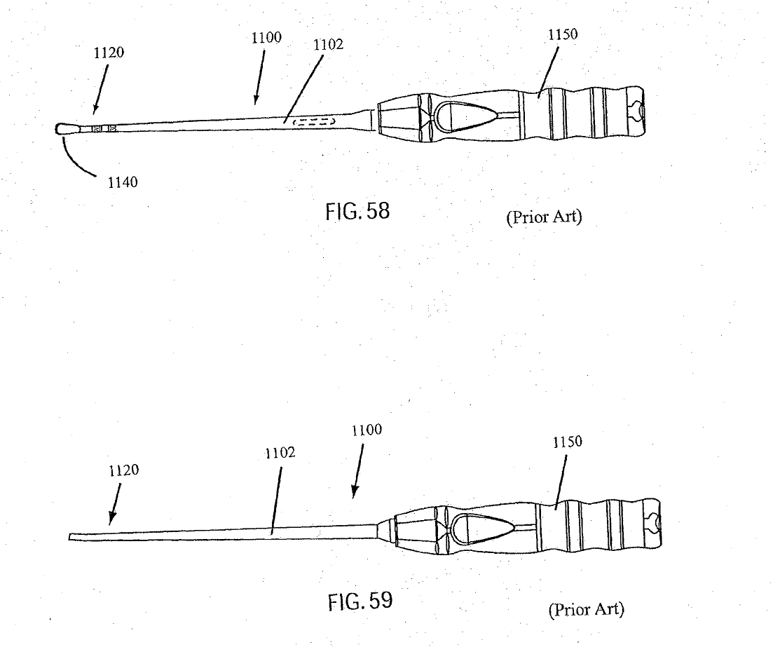

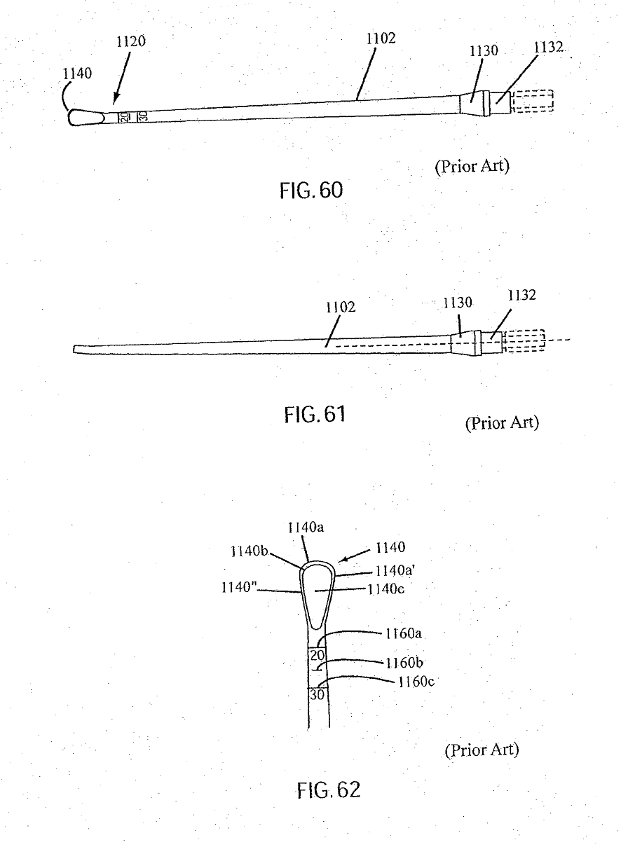

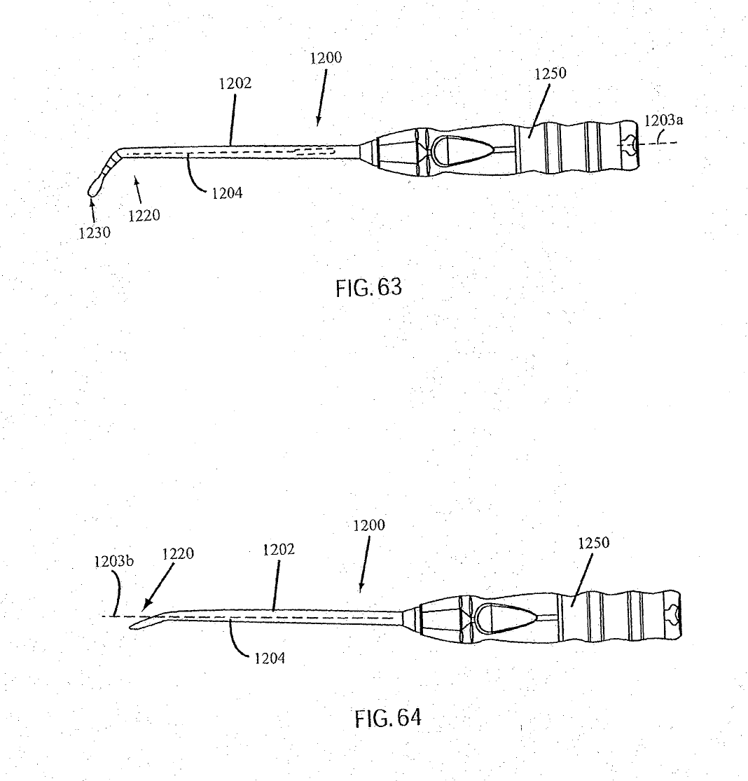

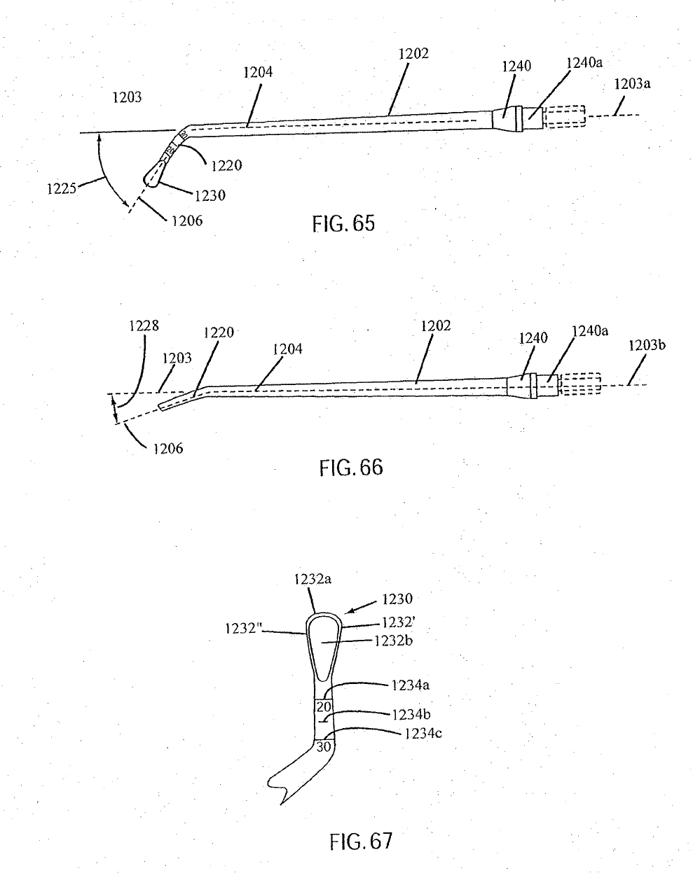

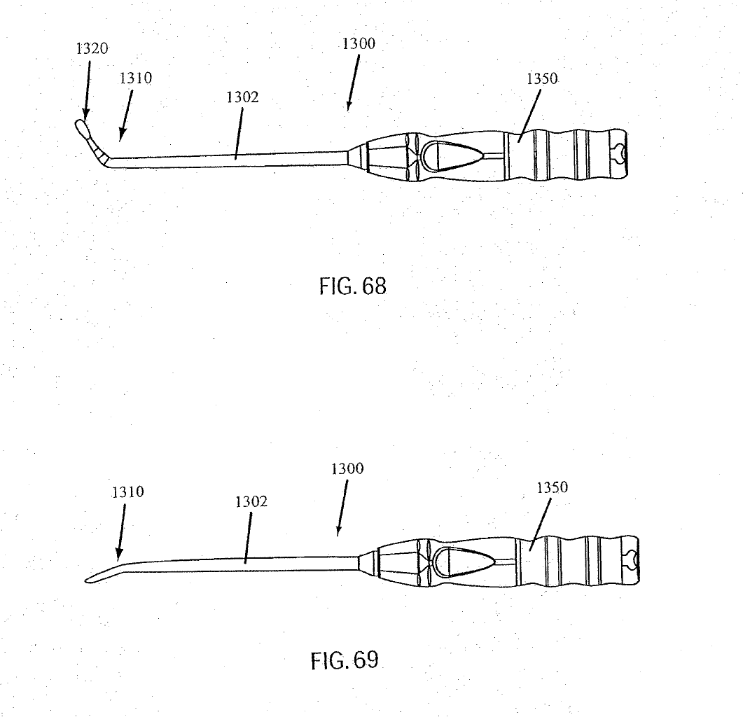

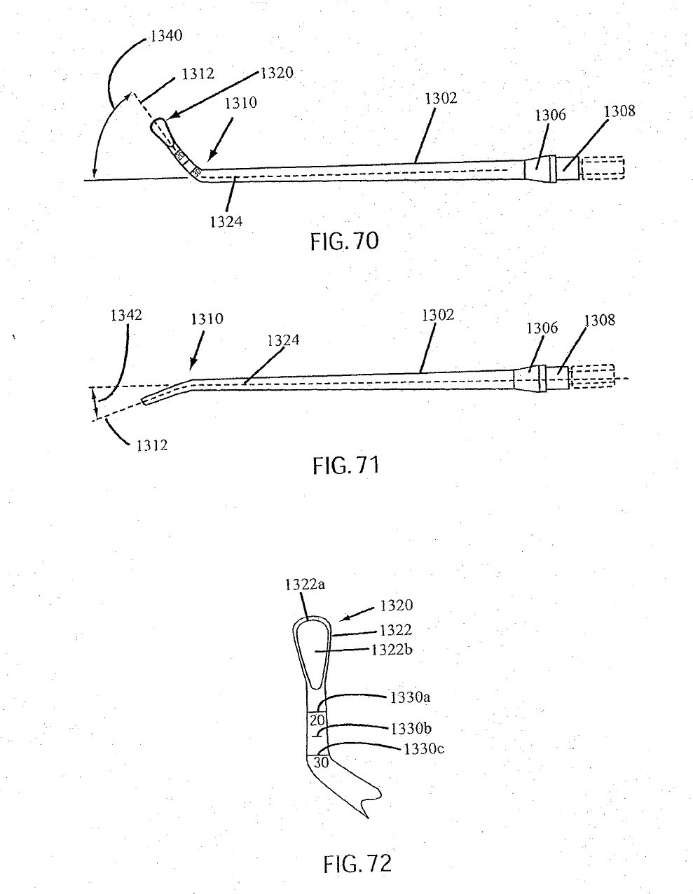

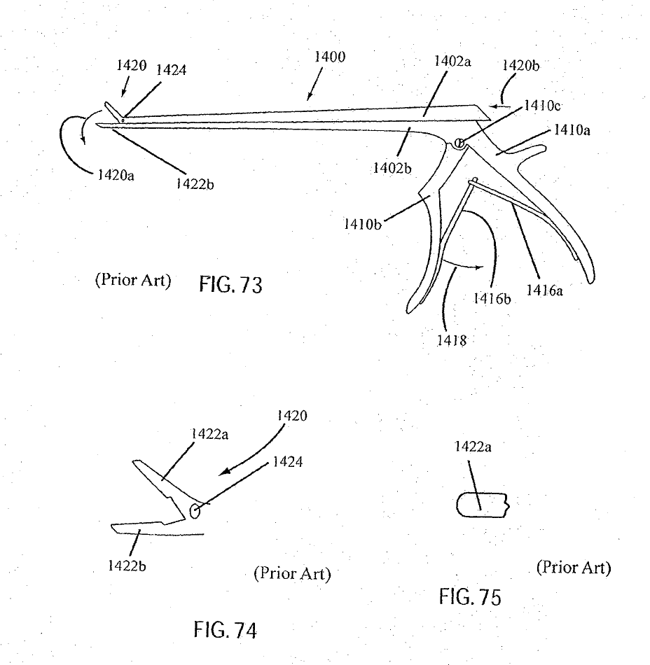

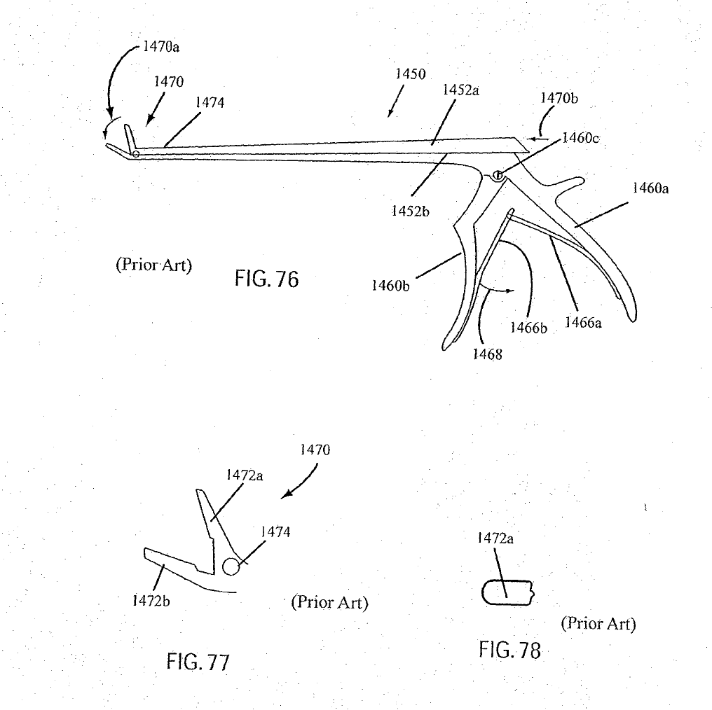

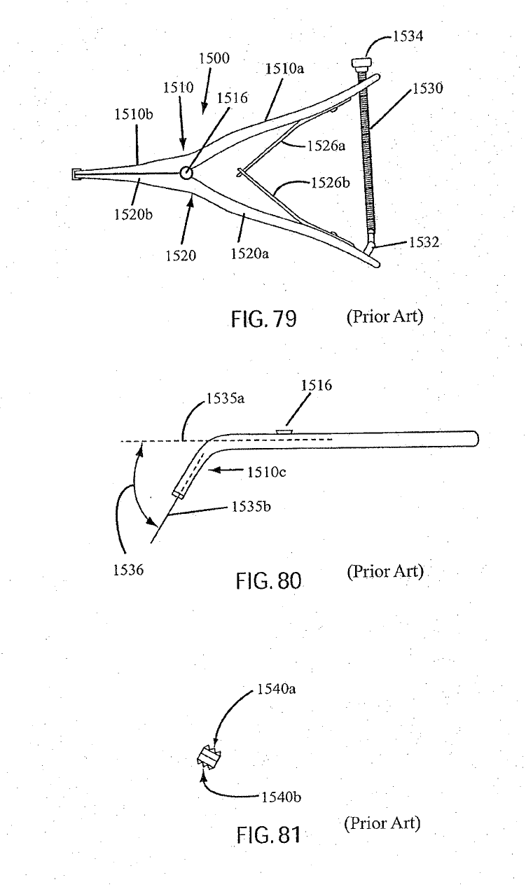

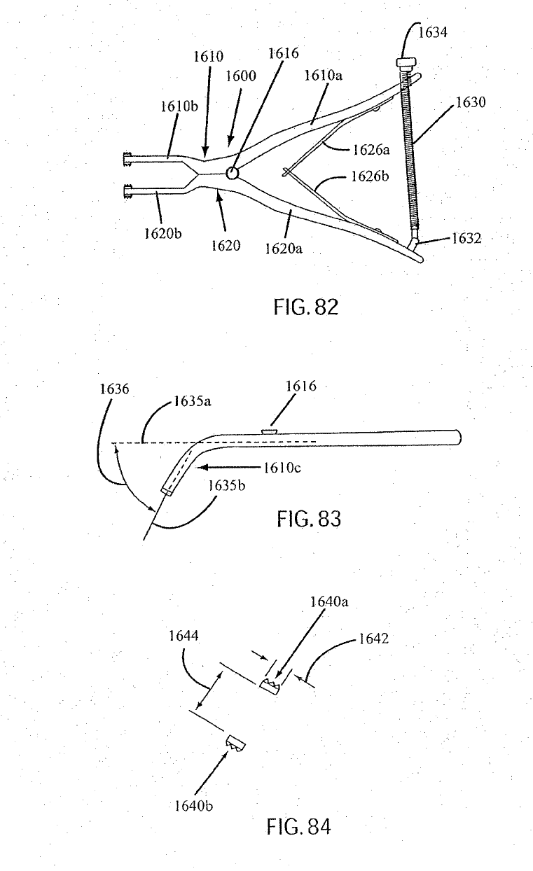

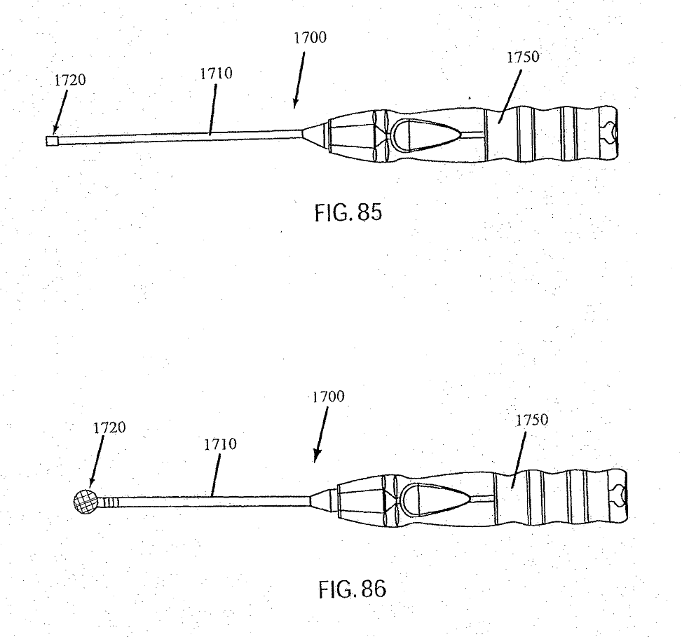

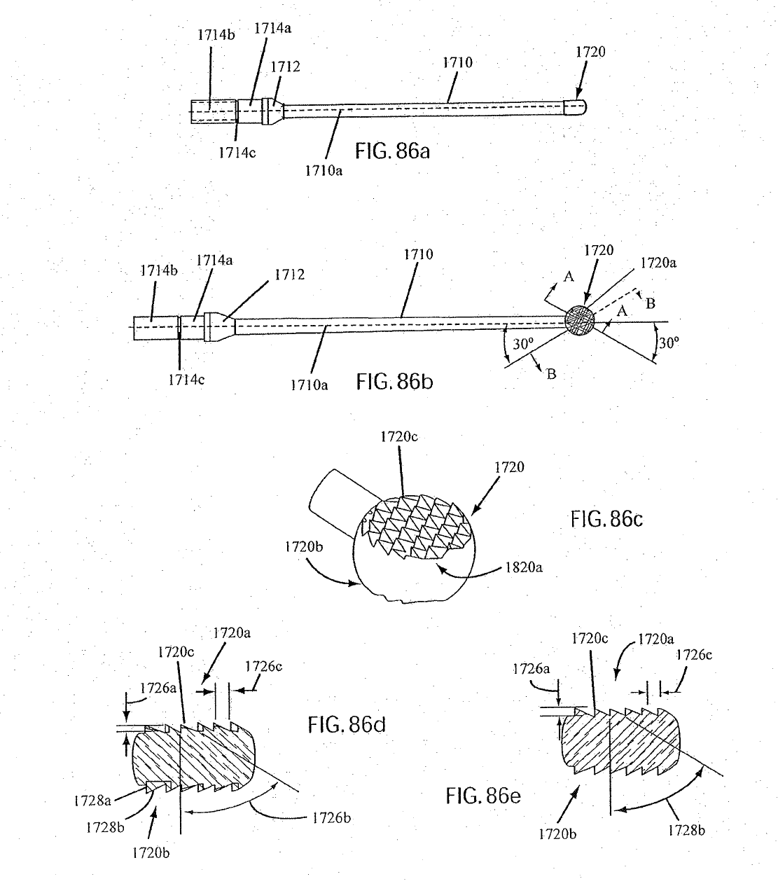

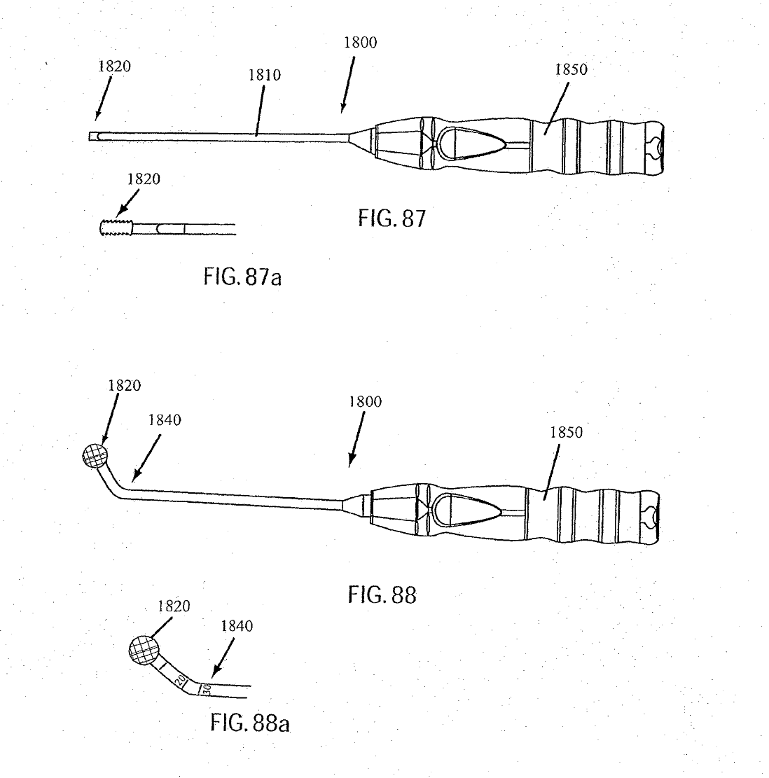

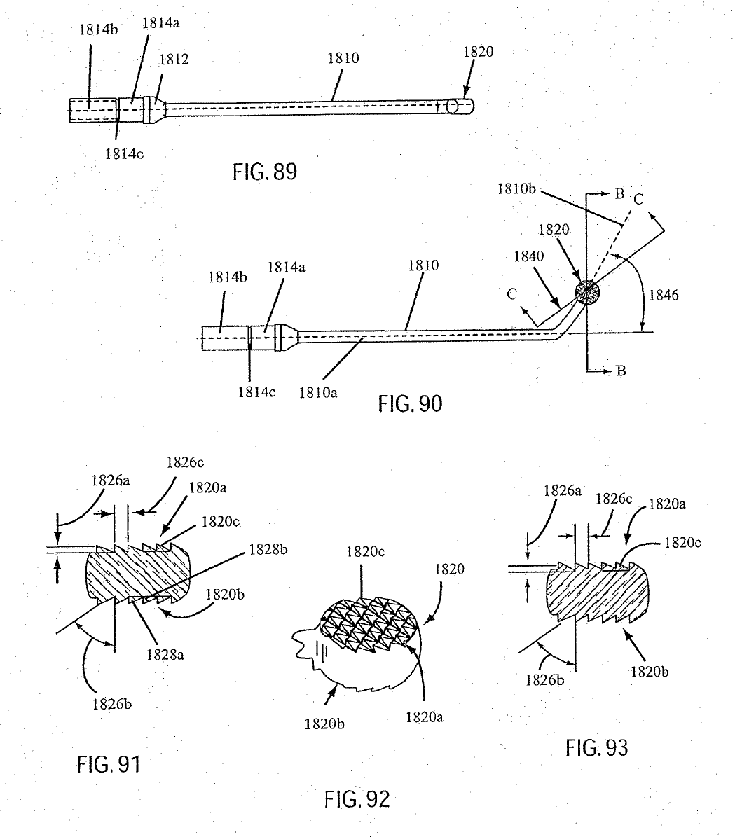

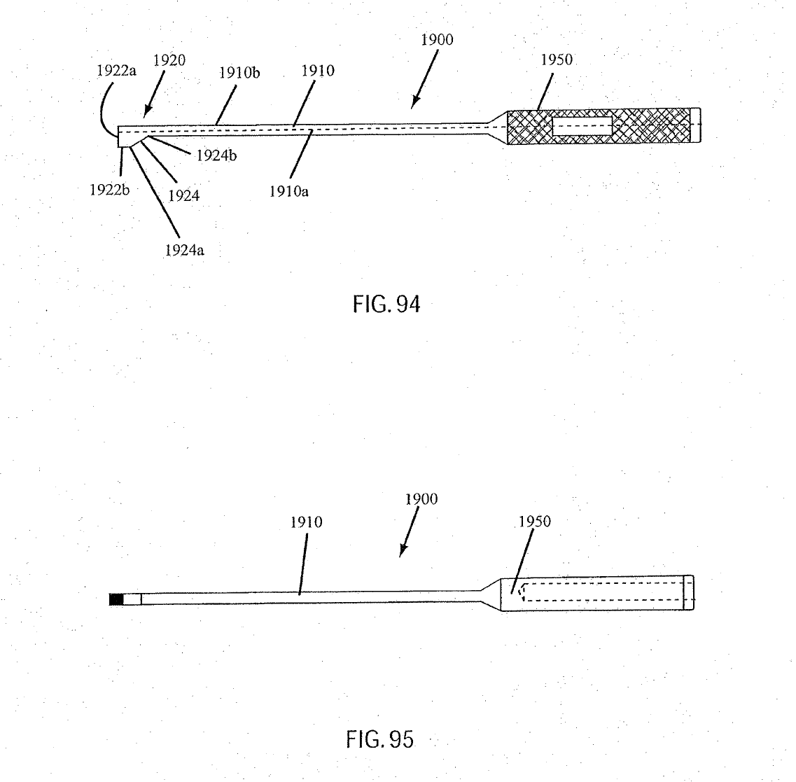

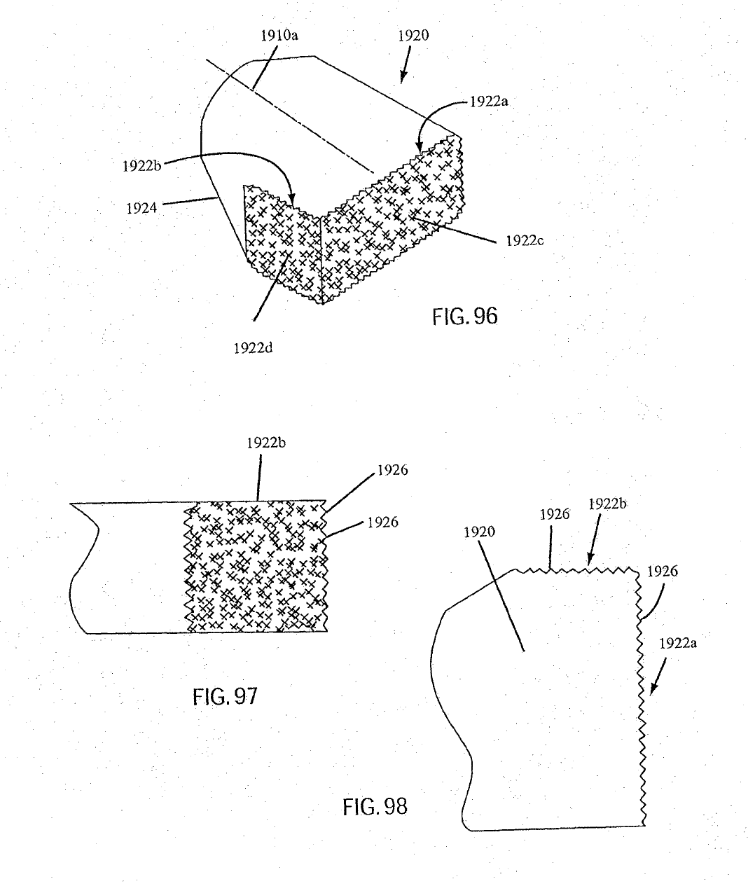

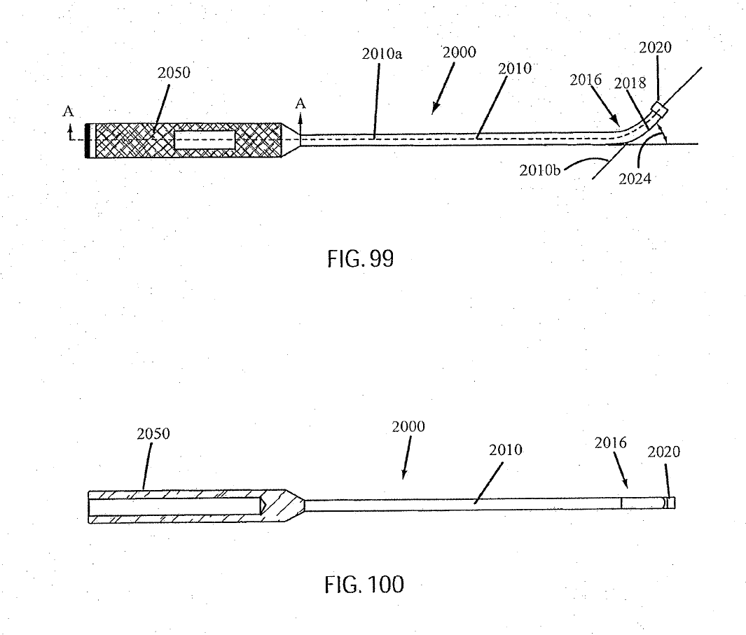

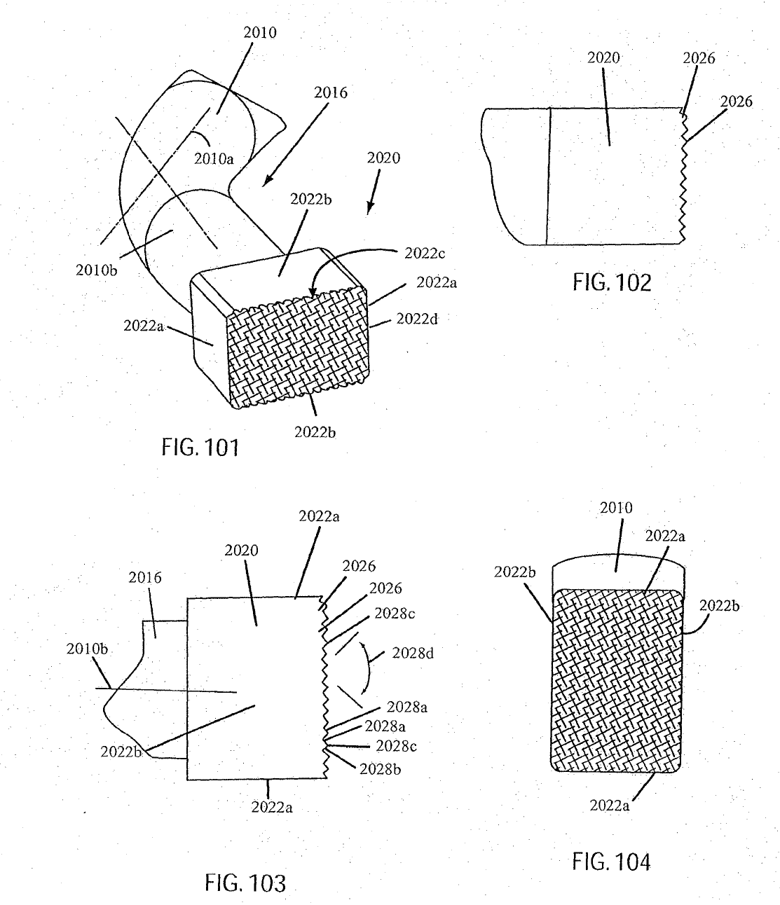

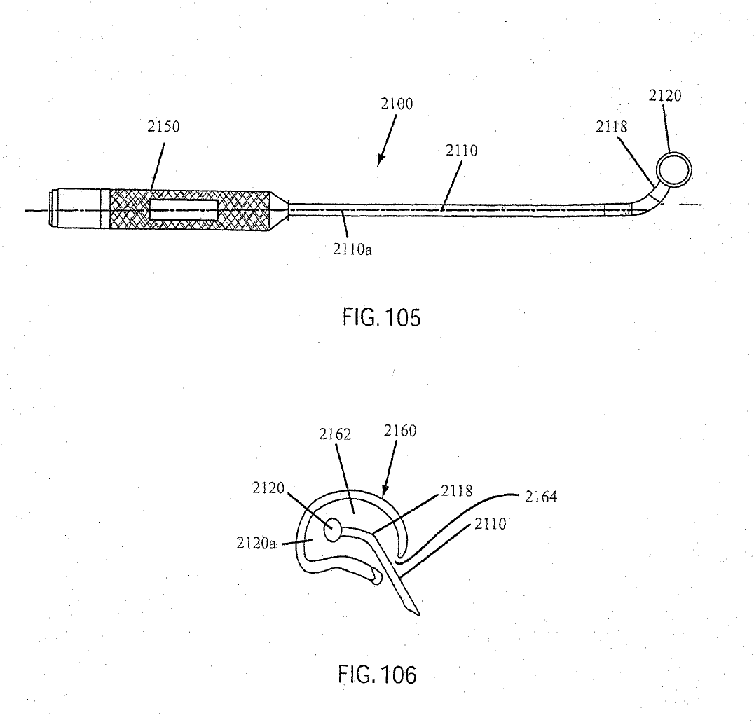

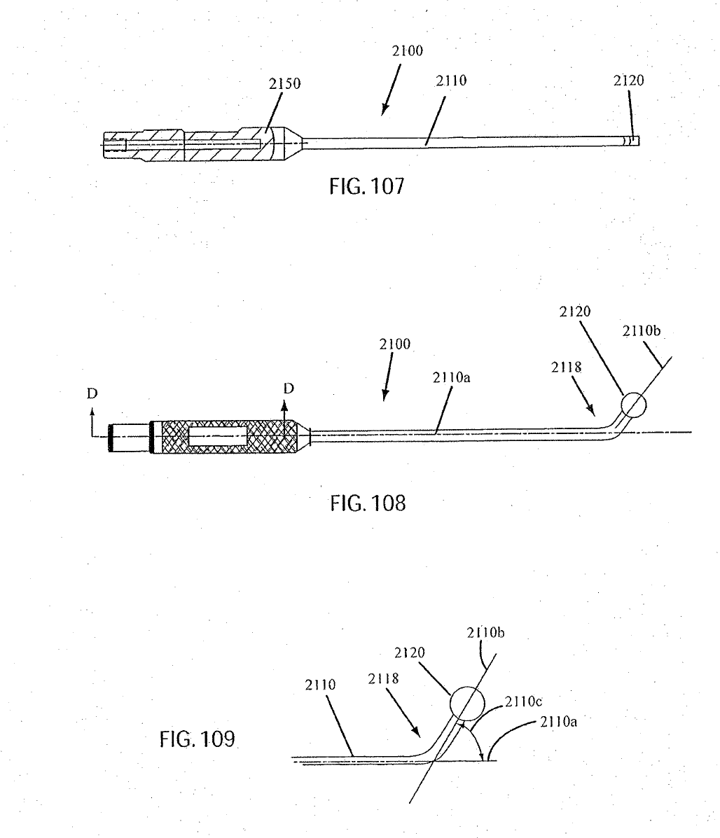

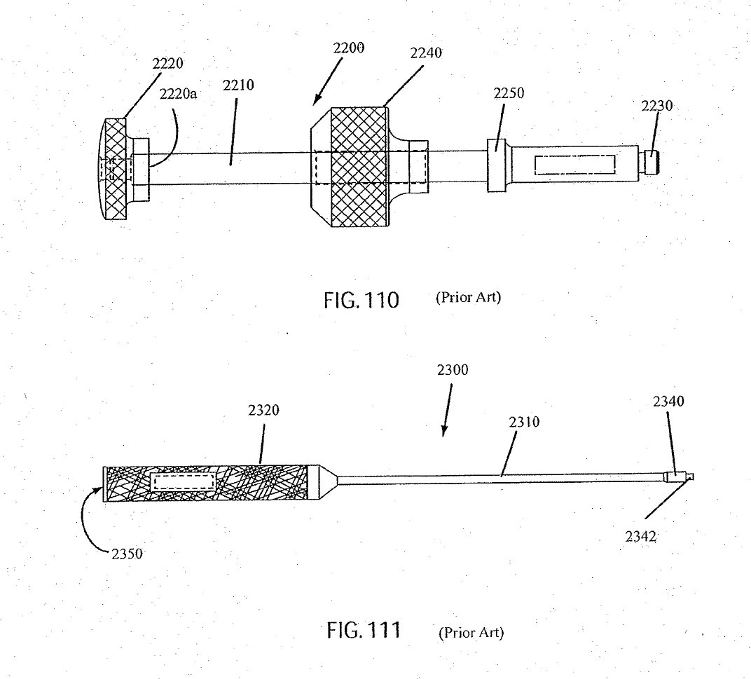

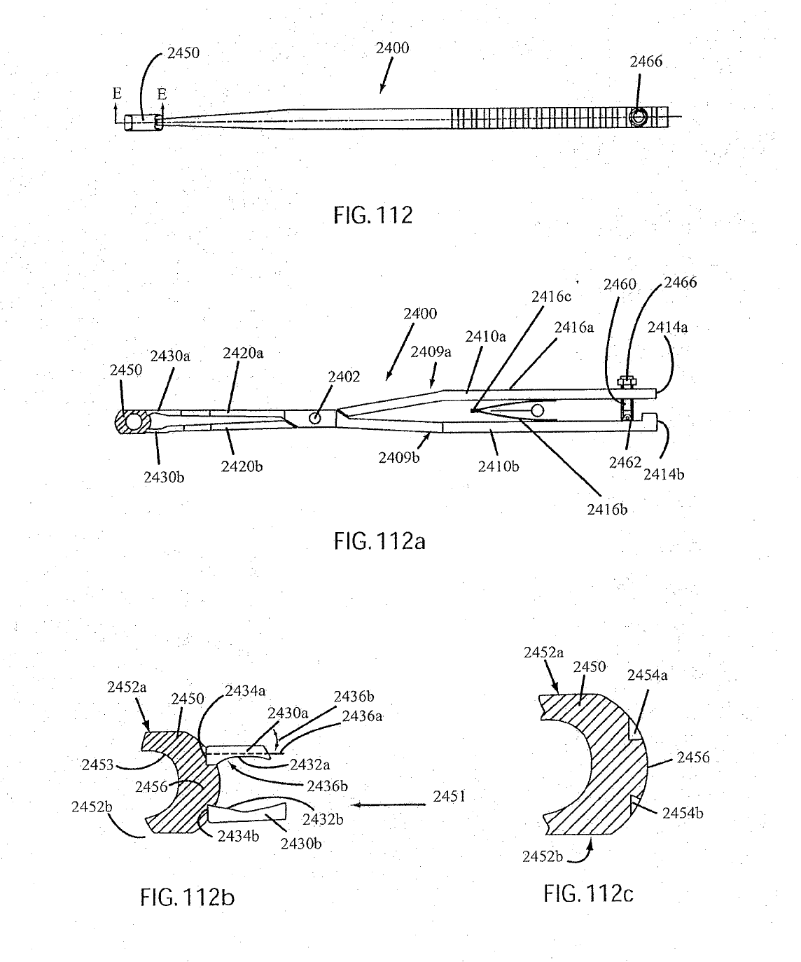

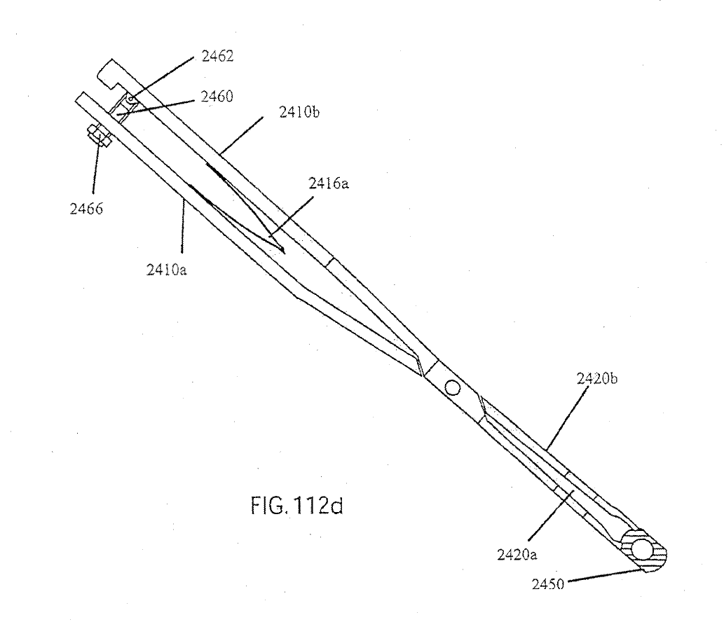

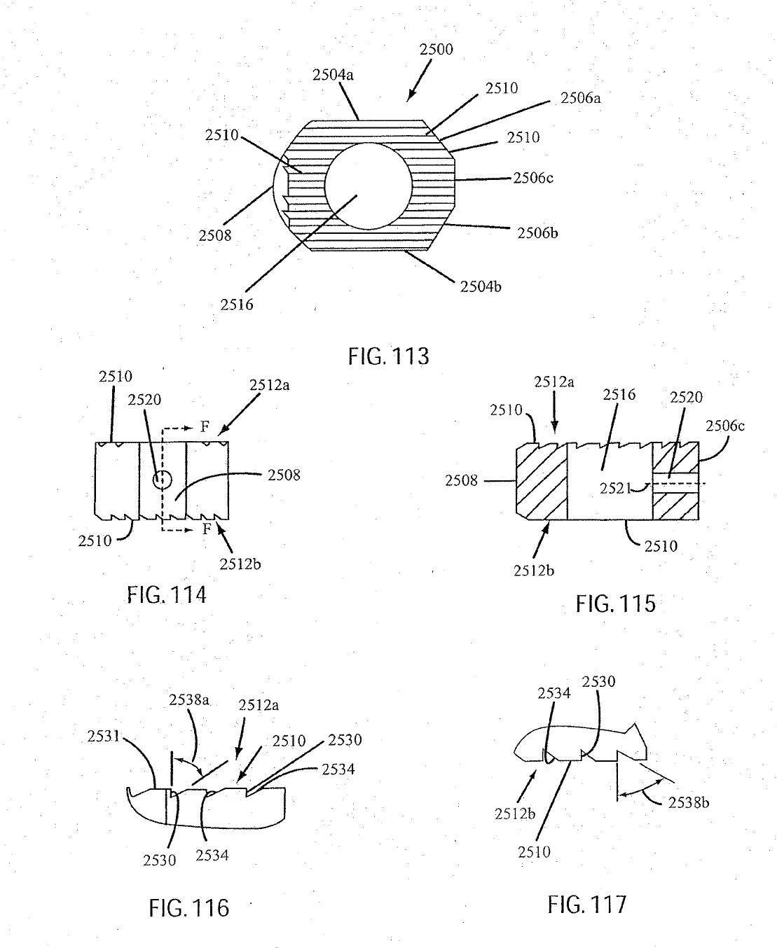

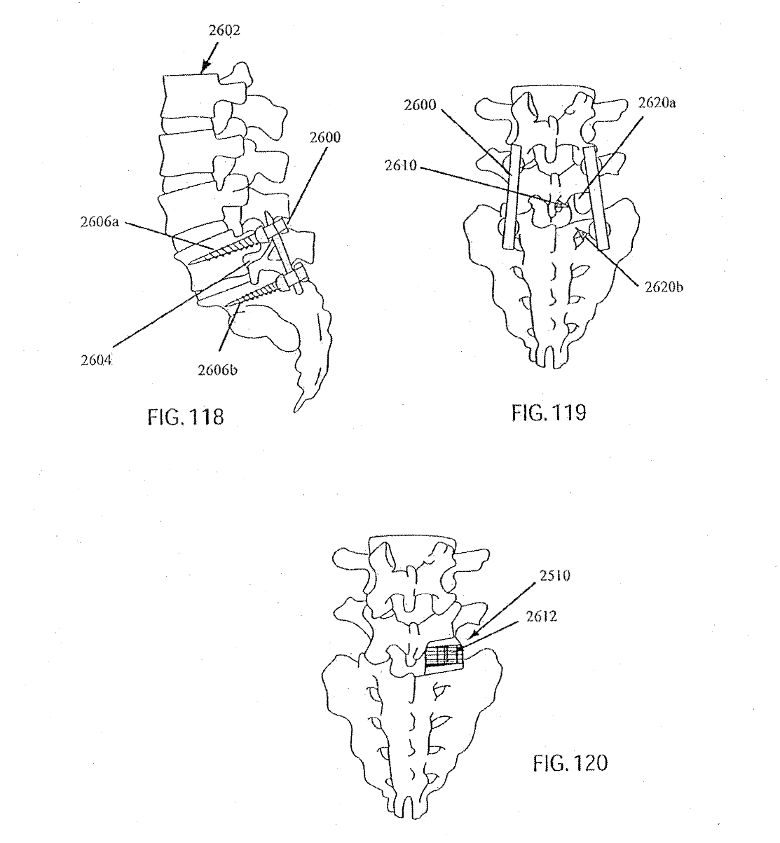

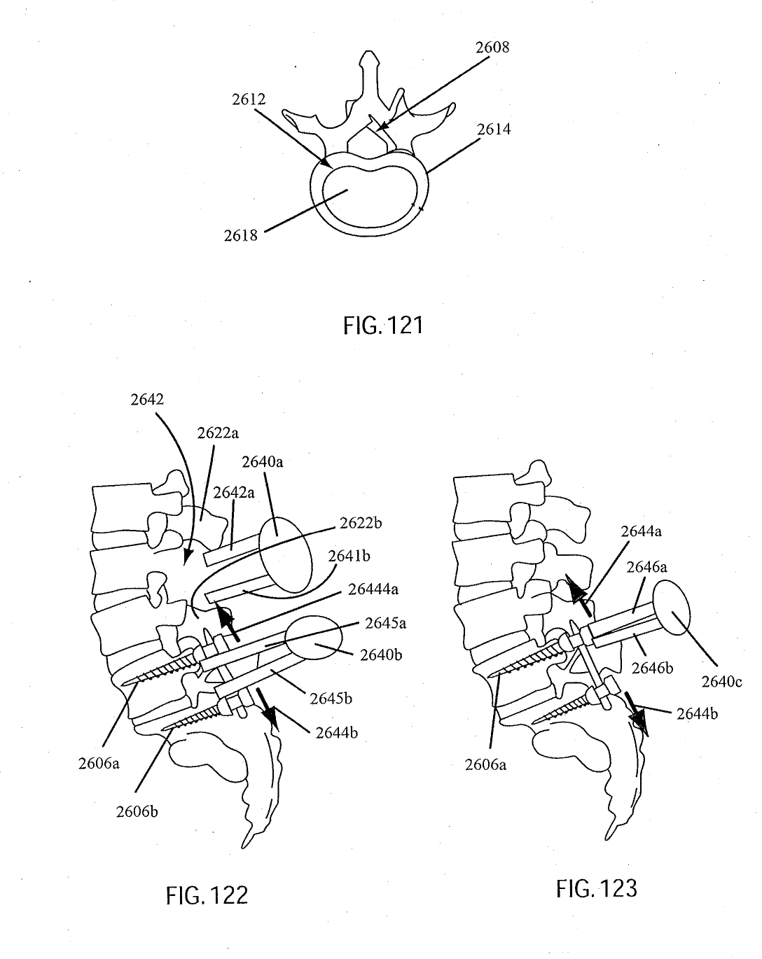

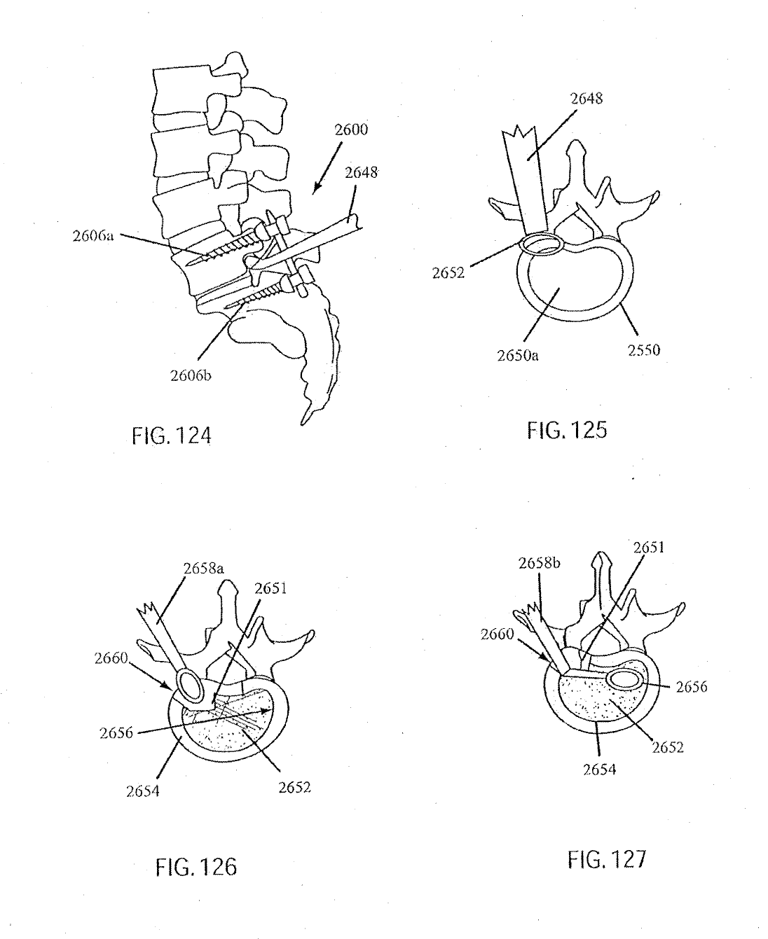

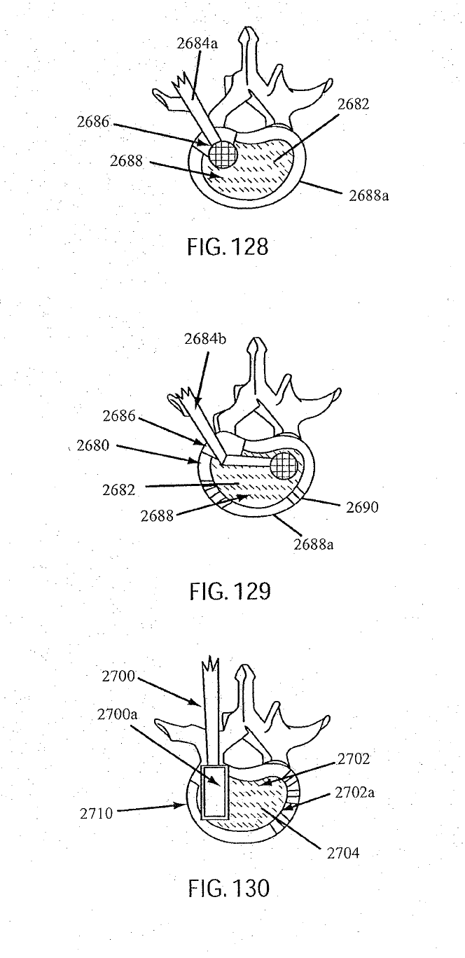

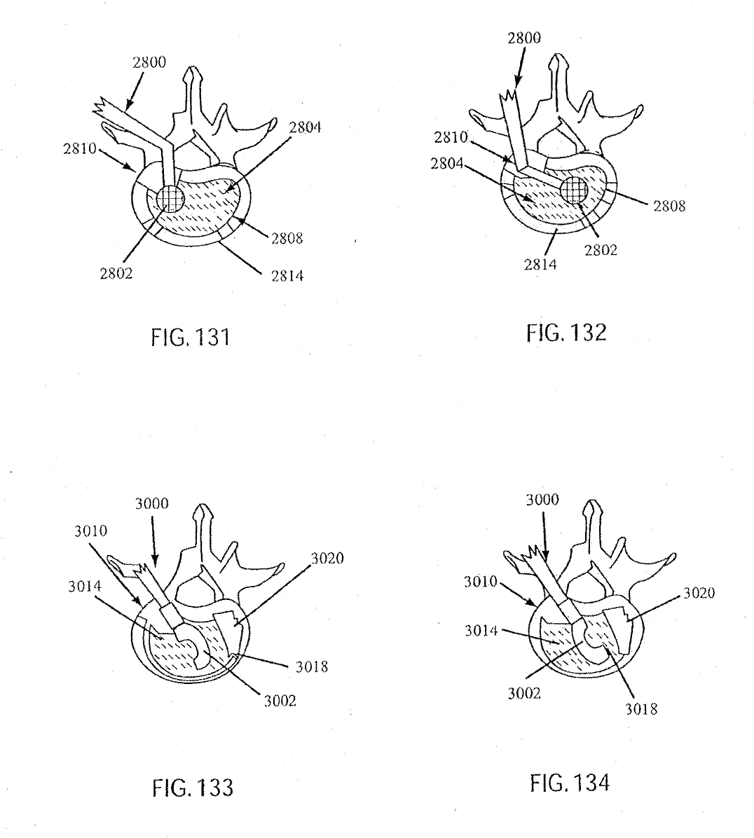

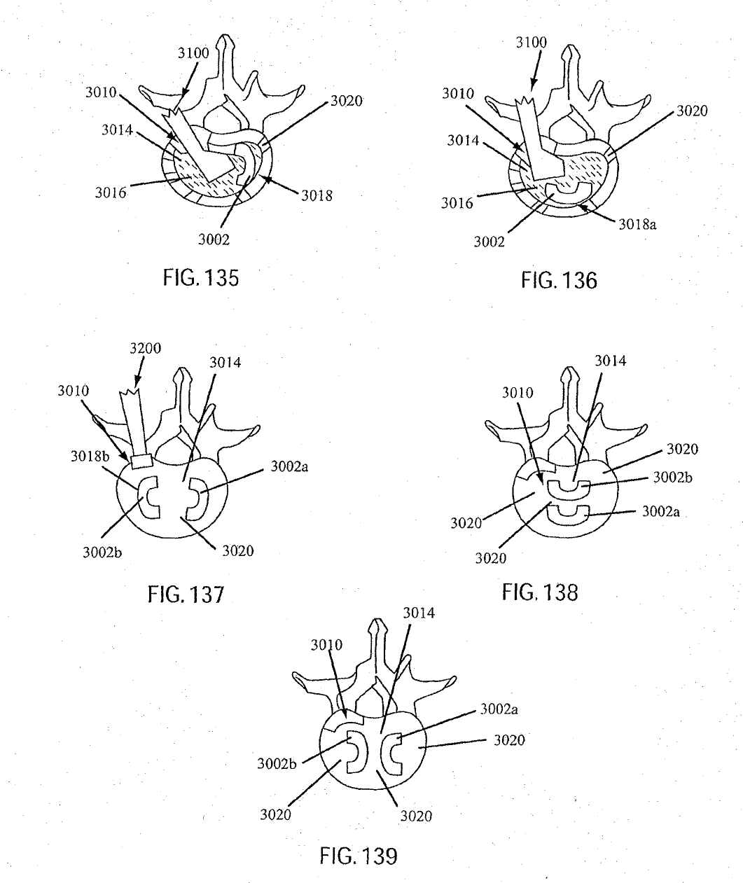

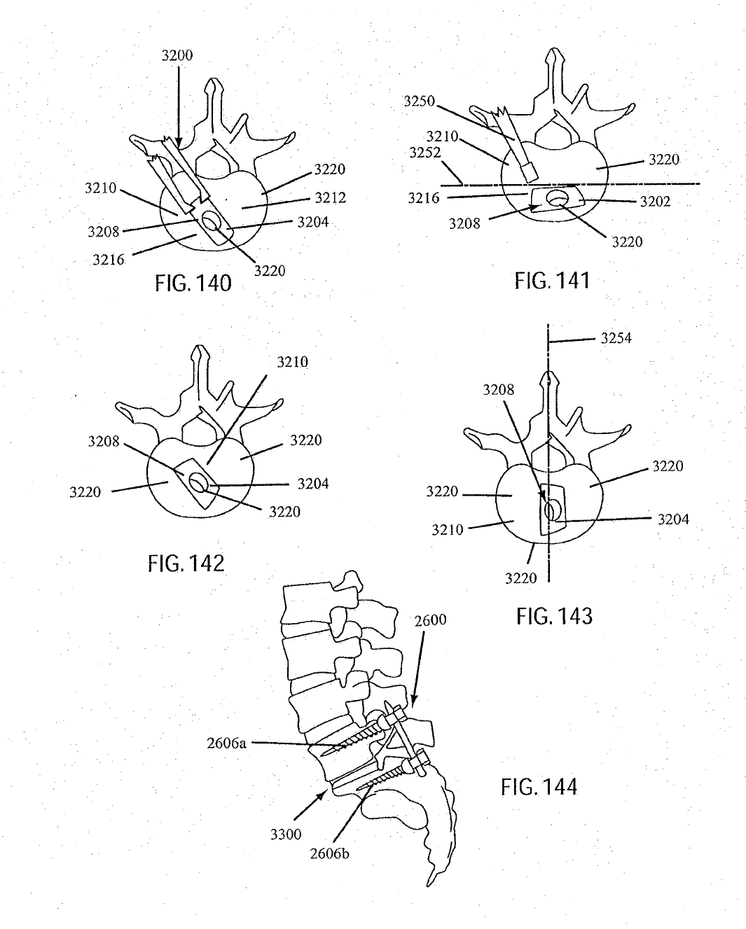

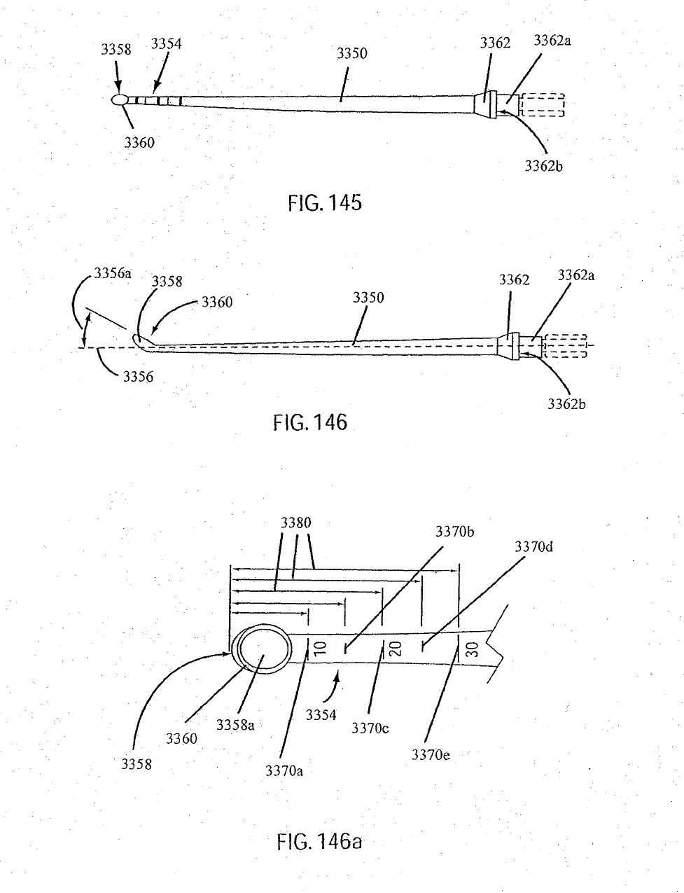

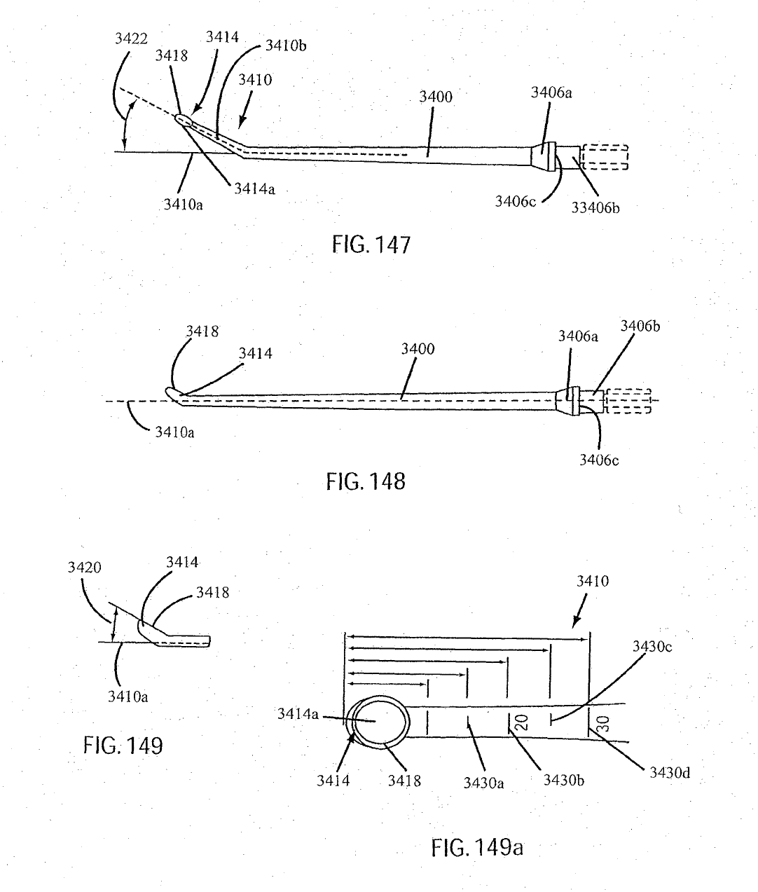

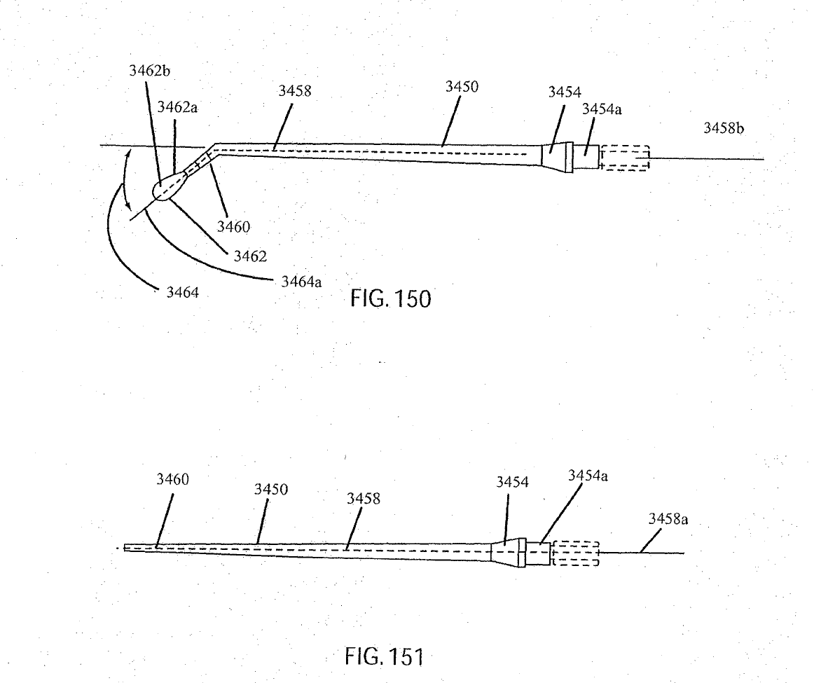

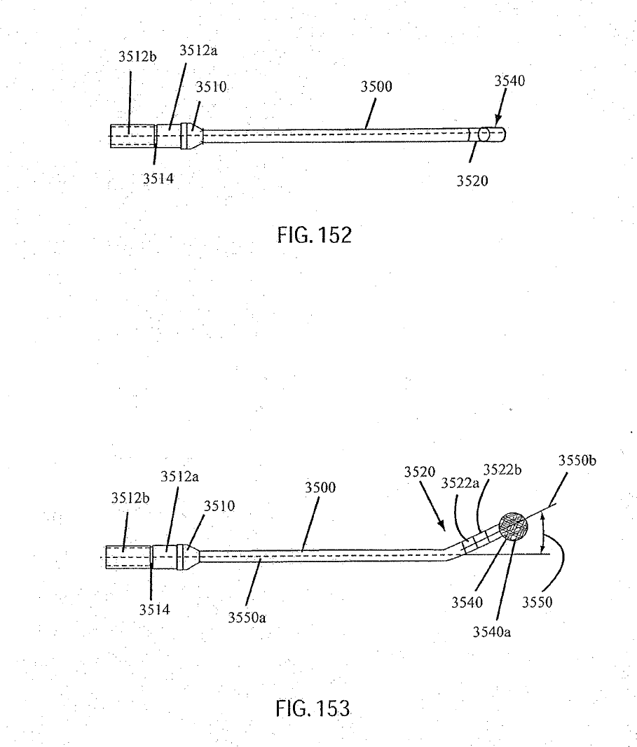

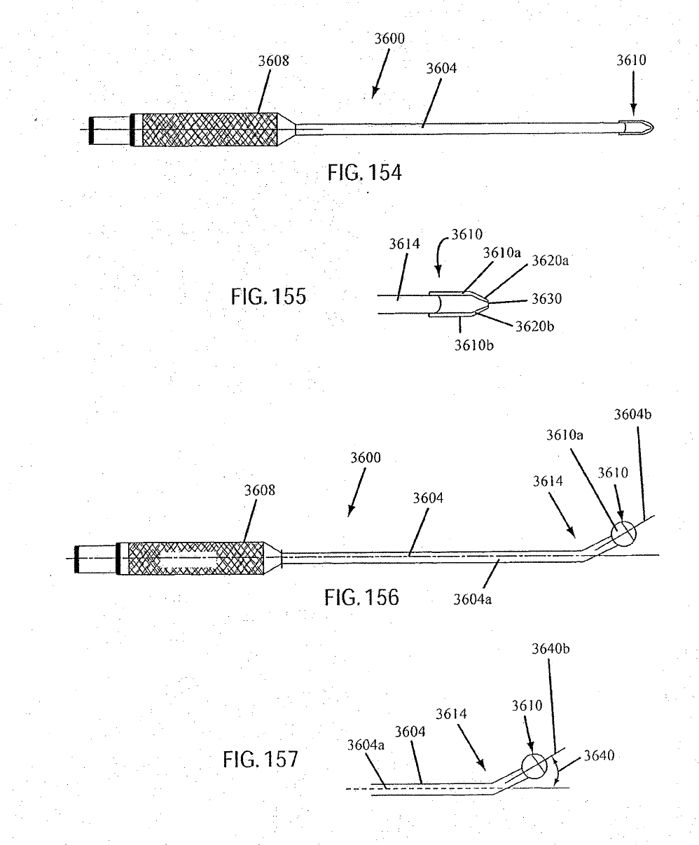

The curette head 920 is oval shaped and has a scraping or cutting surface 922 around the perimeter of the head defining a central through hollow core 920a. The curette head 920 is serrated as shown in detail in FIGS. 49 and 50. The serrations are similar to those on the curette head 800 in the previous embodiment. The serrated surface includes grooves 922a separated by planar surface areas 922b, as shown in FIGS. 49 and 50. The depth 924a of the groove is preferably about 1 mm, and the width 924b of the groove is preferably about 0.5 mm. The shaft head portion 910 includes markings 940a, 940b, 940c and 940d with associated depth numbers for indicating the depth of the shaft head portion 910 into a vertebral cavity.

An alternative embodiment of an inclined curette shaft 3400 is shown in FIGS. 147-149. The curette head 3414 is oval shaped and has a scraping surface 3418 around the perimeter of the curette head defining a central hollow 3414a. The scraping surface 3418 of the curette head 3414 is substantially smooth, as shown in FIGS. 147 and 148. An inclined shaft head portion 3410 of the shaft 3400 is inclined upwardly at an angle 3420 between a central longitudinal axis 3410a through the shaft 3400 and the scraping surface 3418. The angle 3420 is preferably about 30.degree.+−0.5.degree. The head shaft portion 3410 is also inclined toward the right at an angle 3422 between the central longitudinal axis 3410a of the shaft and an axis 3410b passing through the center of the curette head 3414. The angle 3422 is preferably about 45.degree. or less, in contrast to the shaft shown in FIGS. 46-47. The angle of the head shaft portion 3410 of the curette also allows the surgeon to access the contralateral side (the side opposite an opening to a disc space) of a disc space when there is a singular opening to the disc space. A surgeon can choose which instrument's inclined shaft head portion is most desirable with respect to a particular procedure.

The shaft 3400 includes a frusto-conical portion 3406a connected to a cylindrical collar 3406b. The stem 3406b connects to a receiving hole in a handle, such as handle 950 shown in FIGS. 44-45. Similar to the shaft 902 and handle 950 connection shown in FIG. 44, an end wall of the handle 950 abuts an end wall 3406c of the frusto-conical portion 3406a of the shaft 3400.

The inclined head shaft portion 3410 of the curette instrument may also have depth markings 3430a-3430d as shown in FIG. 149a. The depth markings 3430a-3430d permit a surgeon to assess the depth in a disc space while manipulating the curette instrument, or other instruments such as a trial or a rasp. Marking 3430a indicates a 15 mm depth of the curette shaft head portion, similarly, marking 3430b is for 20 mm, marking 3430c is for 25 mm, and marking 3430d is for 30 mm depths. The markings 3430a-3430d indicate to a surgeon the depth of the curette in a vertebral disc space from the distal end of the curette head 3414 to the marking, as indicated by the dimension lines in FIG. 149a.

The shaft shown in FIGS. 147 and 148 may also have a head shaft portion inclined to the left as used by a surgeon with similar features as the shaft 3400 shown in FIGS. 147-148. The left inclined shaft may also have an oval shaped curette head having a substantially smooth scraping surface around the perimeter of the curette head, and a shaft head portion inclined upwardly. The shaft inclined to the left may also be inclined 45 degrees or less similar to the inclined right shaft head portion 3410 at angle 3422 shown in FIG. 147.

A serrated cup curette instrument 1000 having a left inclined head shaft portion 1010 as would be used by a surgeon is shown in FIGS. 51-57. Similar to the previous embodiment of the right inclined curette instrument 900 shown in FIGS. 44-50, the present left inclined embodiment 1000 includes a shaft 1002 connected to a handle 1050 at a proximal end of the instrument, which is essentially the same as prior art handle 806 shown in FIG. 41, and the shaft head portion 1010 at a distal end opposite the handle 1050. The shaft head portion 1010 includes a serrated cup curette 1020. The left inclined curette 1020 allows the surgeon to access the contralateral side of a disc space through a single opening in the disc space.

The shaft head portion 1010 is inclined from a central axis 1005 of the shaft 1002 at a angle 1025 between the axis 1005 and an axis 1028 along the center of the shaft head portion and curette 1020. The angle 1025, as shown in FIG. 53, is preferably about 55.degree.+−0.5.degree. The curette head 1020 is inclined upwardly as shown in FIG. 55. The angle 1075 is between the axis 1005 and a scraping or cutting surface 1022 of the curette head 1020. The angle 1075 is preferably about 30.degree.+−0.5.degree.

The curette head 1020 is oval shaped and includes a serrated surface 1022 defining a perimeter of a central through hollow core 1022b. The surface 1022 has a plurality of grooves 1022a spaced from one another by planar surface areas 1022b. The grooves 1022a have a depth 1024a which is preferably about 1 mm, and a width 1024b which is preferably about 0.5 mm. The shaft head portion 1010 of the shaft 1002 includes indicator markings 1040a, 1040b, 1040c and 1040d with associated depth numbers for indicating the depth of the shaft head portion 1010 into a vertebrae cavity.

A prior art surgical instrument 1100 includes a shaft 1102 having a shaft head portion 1120 at a distal end thereof, FIGS. 58-62. The shaft 1102 is connected to a handle 1150 at a proximate end, which is essentially the same as prior art handle 806 shown in FIG. 41, opposite the shaft head portion 1120. The shaft head portion 1120 includes a curette 1140 having an arcuate wall 1140a contiguous with side walls 1140a′ and 1140a″ defining a perimeter of a closed loop and a central through opening 1140c. The walls 1140a, 1140a′ and 1140a″ define a top surface 1140b, FIG. 62, and a bottom surface (not shown). The top surface 1140b is representative of the bottom surface of the loop curette 1140. Both the top surface 1140b and the bottom surface may be used for scraping and cutting vertebrae disc material. The head shaft portion 1120 of the shaft 1102 includes markings 1160a, 1160b and 1160c with associated depth numbers for measuring the depth of the shaft head portion 1120 in a vertebral cavity. The shaft 1102 includes a frusto-conical proximal end 1130 with a cylindrical collar 1132 extending therefrom for connecting to the handle 1150 as described in previous instrument embodiments.

A loop curette surgical instrument 1200 is inclined in two different directions relative to planes defined by the shaft longitudinal axis 1204, FIGS. 63-67, according to an embodiment of the present invention. The instrument includes a tapered shaft 1202 that narrows as it approaches the head portion 1220 and having a handle 1250 attached at a proximal end, which handle is essentially the same as prior art handle 806 shown in FIG. 41, and a shaft head portion 1220 at a distal end of the shaft 1202. The shaft head portion 1220 includes a loop curette 1230 which is inclined relative to the axis 1204 in two directions in two different planes 1203a and 1203b, FIGS. 63 and 64, respectively. In FIG. 63, the shaft 1202 has a longitudinal axis 1204. The axis 1204 defines a first plane 1203a that is normal to the plane of the drawing sheet. In FIG. 64 the plane 1203a is in the plane of the drawing sheet normal to the orientation of the plane 1203a in FIG. 63. In FIG. 64, the axis 1204 defines a second different plane 1203b normal to the drawing sheet and normal to the plane 1203a. In FIG. 63, plane 1203b lies in the plane of the drawing sheet normal to the plane 1203a. The planes 1203a and 1203b thus are normal to each other. The terms “left” and “right” as used herein refer to relative orientation of the shaft head portion relative to the shaft longitudinal axis in a so called horizontal plane represented by plane 1203b, FIG. 66. This orientation is shown in FIG. 65 as being to the left when the shaft head portion is angled toward the bottom of the drawing sheet and to the right when the shaft head portion is angled toward the top of the drawing sheet, FIG. 65.

The term left refers to the relative orientation of the shaft head portion to the shaft longitudinal axis when looking along the longitudinal axis from the handle to the working head end of the shaft. Thus, in FIG. 65, the shaft head portion 1220 is bent at an angle that extends to the left of the axis 1204 and to the left of the plane 1203a (normal to the drawing sheet) when looking toward the head end. This bend of the shaft head portion is stated herein to be in a horizontal plane with respect to others of the embodiments described herein of tools with bent or angled shaft head portions.

The term “right” is meant to include the orientation of the shaft head portion that is angled to the right opposite to that of the head shaft portion 1220 of FIG. 65 or toward the top of the drawing sheet. The head angle to the left and right thus are both angled into and out of the plane of the drawing sheet in the plane 1203b, FIG. 66.

The term upwardly and downwardly as it is used with the various embodiments herein refer to the relative orientation of the shaft head portion to the plane 1203b, FIG. 66. The head portion 1220, FIG. 66 is angled downwardly in FIG. 66 toward the bottom of the drawing sheet. The plane 1203b thus represents a horizontal plane. These left, right and up and down terms represent orientations that are discussed herein in respect of the various embodiments employing angled shaft head portions of the instrument shafts.

The shaft 1202, FIGS. 65 and 66, is tapered toward it's distal end. The shaft has a frusto-conical portion 1240 attached to a cylindrical collar 1240a extending proximally and adapted to mate with the handle 1250. In FIG. 65, plane 1203a is normal to the drawing sheet and should be considered as a vertically oriented plane and plane 1203b is in the plane of the drawing sheet and should be considered as a horizontally oriented plane. In FIG. 66, plane 1203b is normal to the drawing sheet and plane 1203a is in the plane of the drawing sheet.

In FIG. 65, the shaft 1202 head portion 1220 is inclined to the left relative to the longitudinal axis 1204 (looking to the left in the figure) in the plane 1203b. The inclination is defined by angle 1225, preferably about 55.degree., of the curette 1230 longitudinal axis 1206, FIG. 65, to the shaft longitudinal axis 1204. The shaft head portion 1220 is also inclined downwardly relative to the plane 1203b (normal to the plane of the drawing figure) and to the axis 1204 by an angle 1228, FIG. 66. The angle 1228 of the shaft head portion longitudinal axis 1204 is preferably about 20.degree. to the shaft longitudinal axis 1206.

The curette 1230 has an arcuate wall 1232, and side walls 1232′ and 1232″ defining a tear drop shaped loop with a central opening 1232b, FIG. 67. A top surface 1232a of the wall 1232 is representative of a bottom surface (not shown) and lies in a plane defined by axis 1206 normal to the drawing sheet at angle 1228, FIG. 66. The top 1232a or bottom surface are cutting edges which are used to scrape or cut vertebral material. The shaft head portion 1220 includes markings 1234a, 1234b, and 1234c with associated depth measurements for indicating the depth the shaft head portion in a vertebral cavity.

The left angle 1225, FIG. 65, relative to plane 1203a, and downward angle 1228, FIG. 66, relative to plane 1203b of the looped curette 1230 assists the surgeon in the preparation of the L5/S1 disc space. The L5/S1 disc space has the greatest lordotic angle compared to the other disc spaces in the human vertebrae.

In FIGS. 150 and 151, an alternative shaft 3450 is shown which may be used with the handle 1250 of instrument 1200 shown In FIGS. 63 and 64. The shaft 3450 is frusto-conical tapering toward a distal end and has a frusto-conical portion 3454 at a proximal end attached to a cylindrical collar 3454a extending proximally and adapted to mate with the handle 1250 similar to the shaft 1202 shown in FIGS. 65-66.

The shaft 3450 head portion 3460 is inclined to the left relative to shaft longitudinal axis 3458 (looking toward the left of the drawing sheet along the axis 3458) on a horizontal plane 3458a, FIG. 151. The plane 3458a is defined by longitudinal axis 3458 and lies in the plane of the drawing figure in FIG. 150. This is the surgical use orientation, with the curette facing vertically upwardly. The shaft portion 3460 and the curette 3462 have a longitudinal axis 3464a that is at angle 3464 to the shaft longitudinal axis 3458 and to plane 3458b defined by the longitudinal axis and which plane 3458b is normal to the drawing sheet in FIG. 150. The angle 3464 is preferably about 45.degree. The shaft head portion 3460 extends along its longitudinal axis 3458, FIG. 151. The curette head 3462 has a cutting edge wall 3462a defining a loop with a central opening 3462b. The wall 3462a whose corresponding top and bottom cutting edge surfaces are used to scrape vertebral material as is the curette head 1206, FIGS. 65-67.

In FIGS. 68-72, a loop curette surgical instrument 1300 in an orientation as used by a surgeon has its shaft head portion 1310 inclined to the right and vertically down according to an embodiment of the present invention. The terms right and vertically down refer to planes as described above in connection with FIGS. 63-67. The instrument 1300 includes a shaft 1302 having a handle 1350 attached at a proximal end, and a shaft head portion 1310 at a distal end of the shaft portion 1310 terminating with a curette 1320. The instrument is similar to the previous instrument 1200 shown in FIGS. 63-67, however, the shaft head portion 1310 as used by a surgeon is inclined to the right as shown in FIGS. 68 and 70. The shaft head portion 1310 is also inclined vertically down as shown in FIGS. 69 and 71. In FIGS. 70 and 71, the shaft 1302 has a frusto-conical portion 1306 having a cylindrical collar 1308 extending proximally which mates with the handle 1350, which is essentially the same as prior art handle 806 shown in FIG. 41. The shaft head portion 1310 is inclined right and downwardly from a shaft portion longitudinal axis 1324 in its use orientation along the shaft 1302. The shaft head portion 1310 is at angle 1340, preferably about 55.degree. relative to the shaft 1302 longitudinal axis 1324 in a horizontal plane in its use orientation. The shaft head portion 1310 is also at an angle 1342, preferably about 20.degree., in a vertical plane between the axis 1324 and axis 1312 in its use orientation.

In FIG. 72, the curette head 1320 is tear drop shaped in plan view and includes a peripheral side wall 1322 forming a closed loop defining through opening 1322b. The side wall 1322 has a top cutting surface 1322a and a bottom cutting surface (not shown) for scraping and/or cutting vertebral materials. The shaft head portion 1310 includes markings 1330a, 1330b and 1330c, and associated numbers for indicating the depth of the shaft head portion 1310 from the tip of the wall 1322 when a surgeon inserts the instrument into a vertebral cavity.

In FIG. 73, prior art surgical rongeur instrument 1400 is a straight rongeur including a sliding member 1402a movably positioned on top of a fixed member 1402b. The fixed member 1402b terminates in a fixed lower jaw 1422b at a distal end of the instrument, and has a fixed handle member 1410c at a proximal end of the instrument. A moveable handle member 1410b is pivotally connected to the fixed member 1402b and to the fixed handle member 1410c by a pivot fastener 1410c. Two elongated leaf type springs 1416a and 1416b are between the moveable handle member 1410b and the fixed handle member 1410a to bias the movable handle member 1410b in an open position. The user encounters resilient resistance when squeezing the handles together such that the handles separate under the influence of the springs when released.

A jaw mechanism 1420 is located at the distal end of the instrument 1400 and includes a movable upper tissue cutting jaw 1422a and the fixed lower jaw 1422b. The movable upper jaw 1422a is pivotally connected to the upper member at pivot 1424. The jaw mechanism 1420 is in an open position when the upper jaw 1422a pivoted from the fixed lower jaw element 1422b while the handle member 1410b is at rest in the position shown in the figure. When the movable handle member 1410b is moved in the direction of arrow 1418, the sliding member 1402a moves in the direction of arrow 1420b and the movable upper jaw element 1422a is rotated in direction 1420a closing the jaws.

A prior art surgical rongeur instrument 1450 is shown in FIG. 76 which is similar to the instrument 1400 shown in FIG. 73, however, the instrument 1450 has a fixed lower jaw element which is inclined vertically upwardly relative to the longitudinal axis of the members 1452a and 1452b. The jaw mechanism 1470 includes the lower jaw 1472b and a movable upper jaw 1472a pivotally connected at pivot 1474, as shown in more detail in FIG. 77. The lower jaw is also inclined vertically upwardly. The jaw mechanism operates the same as the instrument 1400. The upper jaw 1472a is biased open in relation to the lower jaw element 1472b. When the movable handle member 1460b is displaced by a surgeon toward the fixed handle member 1460a in direction 1468 by pivoting about pivot fastener 1460c, upper sliding member 1452a slides on a lower member 1452b in direction 1470b. The sliding member 1452a closes the upper jaw element 1472a relative to the lower jaw 1472b.

In FIGS. 79-81, prior art lamina spreader surgical instrument 1500 includes elongated upper arm 1510 and lower arm 1520. The upper arm 1510 includes an upper handle 1510a and an upper jaw 1510b. The lower arm 1520 has a lower handle 1520a and a lower jaw 1520b juxtaposed with the respective upper handle 1510a and upper jaw 1510b. The upper arm 1510 and lower arm 1520 are pivotally connected to a pivot fastener 1516. The upper and lower handles 1510a and 1520a, respectively, are in spaced relation to each other. The upper and lower jaws 1510b and 1520b are adjustable in a spaced relation to each other and have a closed position where opposing jaw surfaces contact one another, FIG. 79. A threaded adjustment rod 1530 is attached to the lower handle portion 1520a at attachment point 1532, and passed through a hole (not shown) in the upper handle 1510a.

Two leaf springs 1526a and 1526b are attached at one end to the respective upper and lower handles 1510a, 1520a, and at an opposite end to each other. The springs 1526a and 1526b bias the handles 1510a and 1520a apart and the jaws 1520b and 1520b together in the closed position. When a surgeon squeezes the handles 1510a, 1520a together, the jaw members 1510b, 1520b are opened to a desired spaced position and then set to this position by rotating knob 1534 threaded to the rod 1530 until the knob abuts the handle 1510a. The jaws are thus locked in this open position for urging the engaged vertebrae apart. This locks the spreader instrument to the distracted vertebrae. Thus, the upper and lower jaw portions are spaced apart to a desired distraction distance as selected by the surgeon.

The upper and lower jaws 1510b and 1520b are inclined to the left relative to the upper and lower arm longitudinal axes in the use orientation, FIG. 80. This inclination of the arms allows improved access to a vertebral cavity. The angle 1536, which may be 60.degree.+−0.3.degree., is between a central axis 1535a along legs 1510, 1520 and a central axis 1535b along an inclined segment 1519c of the jaw portions 1510b and 1520b. A distal end of each of the jaws 1510b, 1520b have bone gripping roughness such as teeth 1540a and 1540b, respectively, extending outwardly and opposite one another to enhance gripping of the lamina.

In FIGS. 82-84, an embodiment of an offset spinal lamina spreader surgical instrument 1600 according to an embodiment of the present invention includes an upper arm 1610 and a lower arm 1620. The upper arm 1610 includes an upper handle 1610a and an upper jaw 1610b opposite the upper handle 1610a. The lower arm 1620 includes a lower handle 1620a and a lower jaw 1620b opposite the lower handle portion 1620a. The upper arm 1610 and lower arm 1620 are pivotally connected to a pivot fastener 1616. The upper and lower handles 1610a and 1620a, respectively, are spaced apart. The upper and lower jaws 1610b and 1620b are also spaced apart when the handles 1610a and 1620a are in their at rest state. The jaws, in their at rest position, are a distance 1644 from each other, FIG. 84. The distance 1644 is preferably 21 mm to 25 mm.

A threaded rod 1630 is attached to the lower handle 1620a at attachment point 1632, and passes through a hole (not shown) in the upper handle 1610a. Two leaf springs 1626a and 1626b are respectively attached at one end to the upper and lower handles 1610a, 1620a, and at an opposite end to each other. The springs 1626a and 1626b bias the handles 1610a and 1620a apart, and thereby bias the jaws 1610b and 1620b apart in their at rest position. When a surgeon squeezes the handles 1610a, 1620a together to position the jaw members 1610b, 1620b to a desired spaced relation, the knob 1634 is threaded on the rod 1630 and locked against the upper handle 1610a to hold the upper and lower jaws in their set open lamina spreading position. Thus, the upper and lower jaws 1610b, 1620b are spaced apart a distance selected by the surgeon.

The upper and lower jaws 1610b and 1620b are inclined to the left in the use orientation relative to the respective arms 1610a and 11610b longitudinal axes 1635a, FIG. 83, allowing better access to a vertebral cavity. The angle 1636 is between a central longitudinal axis 1635a along legs 1610, 1620 and a central axis 1635b along inclined segment 1610c of the jaws 1610b and 1620b. The angle 1636 is preferably about 60.degree.+−0.3.degree. A distal end of each of the jaw portions 1610b, 1620b have teeth 1640a and 1640b, respectively, extending outwardly and opposite one another to enhance gripping of a lamina. The offset lamina spreader according to an embodiment of the present invention may be used, for example, when vertebral or disc material has been removed.

In FIGS. 85-86e, a straight rasp instrument 1700 according to an embodiment of the present invention includes a shaft 1710 connected to a handle 1750 at a proximal shaft end, and a rasp head 1720 at a distal shaft end, opposite the handle 1750. In FIG. 86, the rasp surface 1720a is shown. In FIGS. 86a and 86b, the shaft 1710 proximal end, opposite the rasp head, has a frusto-conical portion 1712 and a cylindrical collar 1714a extending proximally from the frusto-conical portion 1712. A recess 1714c separates collar 1714a from a second cylindrical collar 1714b. Both collars 1714a and 1714b connect with a mating opening (not shown) in the handle 1750.

The rasp head 1720 is shown in greater detail in FIG. 86c. The rasp head has opposing top and bottom surfaces 1720a and 1720b, respectively. The top surface 1720a is shown in FIG. 86c and is representative of both surfaces 1720a, 1720b. A cross section of teeth 1720c is shown in FIGS. 86d and 86e which depicts a plurality of rearwardly facing teeth 1720c. The teeth 1720c are depicted in greater detail in FIGS. 86d and 86e. The teeth 1720c have a height measurement 1726a which is preferably about 0.039 mm. Each tooth is defined by a vertical wall 1728a at a 90 degree angle from a horizontal plane in the axis 1710a, and an inclined wall 1728b preferably at a 60.degree. angle 1726b from the vertical wall 1728a. The tooth pitch 1726c from one vertical wall to another is preferably about 0.067 mm. The inclined teeth 1720c of the rasp head 1720 provides enhanced scraping and filing of vertebral material.

In FIGS. 87-93, an inclined rasp surgical instrument 1800 according to an embodiment of the present invention is similar to the straight rasp 1700 shown in FIGS. 85 and 86. However, the inclined rasp instrument 1800 has an inclined shaft portion 1840 relative to the longitudinal axis of the shaft 1810, which portion enhances access to the contralateral side of a vertebral disc space. The shaft 1810 includes a handle 1850 attached at the shaft proximal end, and rasp portion 1820 is at the opposite distal shaft end. The shaft 1810 has an inclined portion 1840 relative to the shaft longitudinal axis and which portion terminates in the rasp head 1820. The rasp head 1820 extends at an angle 1846, preferably about 55.degree., from the shaft between a central axis 1810a along the shank 1810 and a central axis 1820b along the inclined portion 1840 of the shaft 1810. The rasp surface 1820a is shown in FIG. 92. The shaft 1810 is shown in more detail in FIGS. 89 and 90. The shaft 1810 proximal end, opposite the rasp head 1820, has a frusto-conical portion 1812 and a collar 1814a extending proximally from the frusto-conical portion 1812. A recess 1814c separates collar 1814a from a collar 1814b. Both collars 1814a and 1814b connect with a mating opening (not shown) in the handle 1850. The inclined teeth 1820c of the rasp head 1820 provides enhanced scraping and filing of vertebral material.

In FIG. 86c, the rasp head 1820 has opposing top and bottom surfaces 1820a and 1820b, respectively. The top surface 1820a is shown in FIGS. 91-93 and is representative of both surfaces 1820a, 1820b. A cross section of teeth 1820c is shown in FIG. 86d. The teeth 1820c are rearwardly facing toward the handle. The teeth 1820c are depicted in greater detail in FIGS. 91 and 93 and have a height measurement 1826a which is preferably about 0.039 mm. Each tooth is defined by a vertical wall 1828a at a 90.degree. angle from a plane through the axis 1810a, and an inclined wall 1828b preferably at a 60.degree. angle 1826b to the vertical wall 1828a. The pitch distance 1826c of the teeth from one vertical wall 1828a to another is preferably about 0.067 mm.

An alternative embodiment of a rasp shaft 3500 having an inclined shaft head portion 3520 is shown in FIGS. 152 and 153 and has a shaft similar to the shaft shown in FIGS. 89 and 90. The shaft 3500 may also be attached to the handle 1850 shown in FIGS. 87 and 88. The shaft 3500 has a proximal end, opposite the rasp head 3540, which has a frusto-conical portion 3510 and a cylindrical collar 3512a extending proximally from the frusto-conical portion 3510. An annular groove 3514 separates collar 3512a from a second cylindrical collar 3512b. Both collars 3512a and 3512b connect with a mating opening (not shown) in the handle 1850. The shaft 3500 inclined head portion 3520 terminates with rasp 3540. The rasp shaft portion 3520 extends at an angle 3550 from the shaft longitudinal axis. The angle 3550 is between a longitudinal axis 3550a along the shaft 3500 and a longitudinal axis 3550b along the inclined portion 3520 of the shaft 3500. Preferably, the angle 3550 is about 45.degree. or less. The rasp 3540 has a surface 3540a, FIG. 153, which is the same as the rasp surface shown in FIGS. 90-93.

The inclined portion 3520 of the shaft 3500 includes depth markings 3522a and 3522b, FIG. 153, representative of a plurality of incremental markings which may be similar to the markings on the inclined portion 3410 of the curette shaft 3400, FIG. 149a. Similarly, the depth markings 3522a and 3522b on the shaft 3500 indicate the depth of the inclined portion 3520 into the intervertebral disc space.

In FIGS. 94-98, a surgical instrument 1900 according to a further embodiment of the present invention has an L-shaped tamp head 1920. The instrument 1900 includes a handle 1950 attached to one end of a shaft 1910 opposite to the tamp head 1920. The tamp head 1920 has a planar end wall 1922a lying in a plane perpendicular to a central axis 1910a of the shaft 1910. A wall 1924 inclined to the central axis 1910a is between the shaft outer surface 1910b, FIG. 94, and a planar side wall 1922b which is preferably contiguous and perpendicular to the end wall 1922a. The junctions 1924a and 1924b between the inclined wall 1924 and the planar side wall 1922b and between the inclined wall 1924 and the outer surface of the shaft 1910 are preferably substantially smooth. The inclined wall 1924 forms a ramp that minimizes tissue catching on the tamp head 1920 during withdrawal from the disc space.

The end wall 1922a of the tamp head 1920 has a roughened surface formed of a diamond pattern grooves 1922c and peaks 1922d forming ridges 1926. A portion of the side wall 1922b, FIG. 96, also has the ridges 1926. The ridges 1926 enhance surface friction and gripping action between the tamp head 1920 and a spinal implant (not shown) to be inserted between adjacent vertebrae.

In FIGS. 99-104, a surgical tamp instrument 2000 according to a further embodiment is shown which includes a shaft 2010 having a handle 2050 attached to one shaft end and a tamp head 2020 attached at the opposite shaft end. The shaft 2010 has a longitudinal axis 2010a. The shaft 2010 has a shaft portion 2016 that has a longitudinal axis 2010b and terminates at tamp head 2020. Portion 2016 axis 2010b is inclined relative to axis 2010a. The angle of inclination 2024 of the shaft portion 2016 longitudinal axis 2010b to shaft 2010 axis 2010a is preferably about 45.degree. The angular relation of the tamp head 2020 orientation to the shaft 2010 facilitates seating a graft implant during spinal surgery providing access to the contralateral side of the vertebral disc space from a remotely located opening on the disc space opposite side.

The tamp head 2020 has two opposing side walls 2022a, and opposing top and bottom walls 2022b. An end wall 2022c of the tamp head 2020 has a rough implant gripping two dimensional array of diamond shaped pyramidal patterned teeth 2026 on surface 2022d, which surface is rectangular but may be square or other geometrical shapes, e.g., polygon such as hexagonal or circular cylindrical, for example. The teeth 2026 have side walls 2028a and 2028b inclined relative to each other and to the end surface plane, forming saw teeth, terminating at apex 2028c, FIG. 103. The angle 2028d subtended by the adjacent teeth side walls 2028a, 2028b, terminating at each root between the adjacent teeth is preferably about 90.degree. The teeth 2026 enhance gripping between the tamp surface 2022d and an implant to resist relative slippage of the tamp to the implant during implant insertion into the disc space.

In FIG. 105, a TLIF (transforaminal lumbar interbody fusion implant trial surgical instrument 2100 is shown. The instrument 2100 includes a shaft 2110 having a handle 2150 attached at a proximal end, and has a disc shaped trial head 2120 attached at a distal end at an inclined shaft portion 2118. The shaft portion 2118 terminates with the trial head 2120. The inclined shaft portion 2118 extends at an angle 2110c from the shaft longitudinal axis 2110a. The longitudinal axis 2110b of the inclined shaft portion 2118 is at angle 2110c, preferably about 55.degree., FIG. 109, to the shaft longitudinal axis 2110a and is. The inclined shaft portion can access a contralateral side 2120a of a vertebral disc space 2162 with respect to a disc space opening 2164.

The inclined shaft portion 2118 and the trial head 2120 are shown within the disc space 2162, FIG. 106. The trial is introduced into the disc space through opening 2164 to the contralateral disc space side 2120a. The trial head 2120 is used to measure the size of the gap between adjacent vertebrae. The fit and size of the trial head 2120 is matched to a corresponding implant.

An alternative embodiment of a trial instrument 3600 is shown in FIGS. 154-157. The instrument includes a shaft 3604 having a handle 3608 connected at a shaft proximal end and a trial head 3610 at the shaft 3604 distal end. The trial head 3610 has upper and lower disc shaped surfaces 3610a and 3610b. In FIG. 155, the head 3610 is chamfered at 3620a and 3620b at it's distal end. The chamfer on the head 3610 provides easier initial access between vertebrae before the full thickness of the trial is reached. A slap hammer (as shown in FIG. 110) may be used to withdraw the trial from the disc space.

In FIGS. 156 and 157, trial instrument 3600 has a shaft 3604 that includes a shaft portion 3614 inclined at an angle 3640 of about 45.degree. to the shaft 3604 longitudinal axis 3604a.

A prior art slap hammer assembly 2200, FIG. 110, includes a shaft 2210 having a knob 2220 attached at one end and a threaded stud 2230 at the opposite end. A sliding weight 2240 rides freely along the shaft 2210 between the knob 2220 and an abutment member enlarged collar 2250. The threaded stud engages a mating threaded end of a trial instrument or other spinal surgical instrument to aid in the insertion or removal of the instrument, if necessary. When the slap hammer and an instrument such as a trial are attached, the sliding weight member 2240 is slid along the shaft 2210 until it impacts the knob 2220. This force is a withdrawal force and is varied as controlled by the surgeon. A variable controlled insertion force is applied to the attached instrument by the surgeon sliding the weight 2240 against the collar 2250.

A prior art posterior implant insertion instrument 2300 is shown in FIG. 111. The instrument 2300 includes a shaft 2310 having a handle 2320 attached at one end. The handle 2320 has a planar impact surface 2350 at it's free end. The shaft 2310 includes a circular cylindrical head 2340 at its other end and which shaft terminates with a threaded stud 2342. The threaded stud 2342 threads into a mating threaded bore in a spinal implant, the head 2340 abutting the implant.

A TLIF spinal implant insertion instrument 2400 is shown in FIGS. 112, 112A, and 112C. Of interest is commonly owned application PCT/US02/34466 corresponding to commonly owned U.S. Patent Application Nos. 60/340,734, 60/372,972 and Ser. No. 10/282,552, all incorporated by reference herein. The instrument 2400 includes two mirror image arms 2409a and 2409b pivotally connected together by pivot fastener 2402 in a scissors configuration. A portion of each of the arms form spaced handles 2410a and 2410b. A distal portion of the arms form opposing jaw members 2420a and 2420b terminating with opposing jaws 2430a and 2430b, respectively, FIGS. 112a and 112b.

In FIG. 112a, the free ends of the handles 2410a and 2410b have insertion force receiving impact surfaces 2414a and 2414b, respectively. A pair of leaf springs 2416a and 2416b are attached to facing sides of the handles 2410a, 2410b, and extend toward one another terminating at a junction 2416c. The springs 2416a, 2416b bias the handles apart and thereby bias the jaws 2420a and 2420b apart. A threaded rod 2460 is attached to handle 2410b and passes through handle 2410a. A threaded knob 2466 mates with the threaded rod 2460 and is threaded to abut and lock with the handle 2410a to hold the jaws 2420a, 2420b and their tips 2430a, 2430b at any desired spaced apart position. Closing the handle spacing closes the spacing between the jaws 2420a and 2420b forming a clamp to thereby grip and clamp the implant 2450 held by the jaws.

The implant 2450 includes mirror image spaced notches 2454a and 2454b, FIG. 112c, which notches have complementary triangular cross sectional shapes as the male jaws to closely receive the jaws, FIG. 112a. The implant 2450 has planar side walls 2452a and 2452b in opposing spaced relation. The jaw tips 2430a and 2430b mate with the notches 2454a and 2454b, FIG. 112b, such that the implant is positively gripped as the jaws are displaced toward each other. Impact forces are transmitted by the free end surfaces 2434a and 2434b of the jaws in a plane normal to the impact force direction 2451. The notches have wall surfaces normal to direction 2451 parallel to the free end surfaces 2434a and 2434b of the insertion jaws to positively receive the impact forces thereon in direction 2451. The notches are aligned with the implant side walls 2452a and 2452b so that the impact forces are applied through the implant side walls 2452a and 2452b to preclude failure of the implant due to such forces, if applied at the central region aligned with the central opening 2453 This maximizes the strength of the implant during insertion.

As the implant is being inserted, the front wall thereof opposite the rear wall 2456 bears the greatest resistance forces as the implant is inserted between the adjacent vertebrae which form a tight fit for the implant in a known manner and which offer considerable resistance to such insertion of the implant. Transmission of the insertion forces through the side walls minimizes potential splintering of the bone during insertion due to stress concentration at the central opening 2453 which might otherwise occur if the forces were applied more centrally.

The jaw tip 2430a has an inclined wall 2432a and a planar end wall 2434a. The jaw tip 2430b similarly includes an inclined wall 2432b in opposing mirror image relation to inclined wall 2432a, and planar end wall 2434b preferably coplanar with wall 2434a. The inclined wall 2432a of jaw tip 2430a, representative of jaw tip 2430b, is at an angle between a longitudinal axis 2436a through the jaw tip 2430a and the inclined wall 2432a. The angle is preferably about 10-25.degree. with respect to the jaw longitudinal axis. Once the implant 2450 is grasped by the jaw tips, the knob 2466 is displaced into abutting relation with an outer surface of the handle 2410a to lock the jaws 2420a and 2420b into position and positively grip the implant.

In FIG. 113, an implant 2500 according to an embodiment of the present invention preferably includes parallel ridges 2510, along the respective top and bottom surfaces 2512a and 1512b, FIG. 114. The ridges on the top surface are oriented at right angles to the ridges on the bottom surface in a manner similar to the implant 400 of FIGS. 14 and 15. The ridges 2510 on the top surface 2512a are preferably at right angles to the ridges 2510 on the bottom surface 2512b, FIGS. 114 and 115. The implant has opposing parallel planar side walls 2504a and 2504b, and posterior end walls 2506a and 2506b inclined to and intermediate the side walls and posterior end wall 2506c. The latter wall is perpendicular to the side walls 2504a, 2504b. Opposite the side walls 2506a, 2506b, 2506c is an arcuate anterior end wall 2508 having a radius from the center of a substantially central circular opening 2516 in the implant. The opening 2516 may be formed by the medullary canal and further finished. The implant has an insertion tool prong receiving bore 2520 on the anterior-posterior axis in the end wall 2506c, FIGS. 114 and 115. The hole 2520 receives a mating insertion prong of an implant insertion tool head (not shown) and if threaded, such as the stud 2342 on insertion tool 2300, FIG. 111.

The ridged top surface 2512a is shown in greater detail in FIG. 116. The ridge 2510 has a planar top surface 2531 with a first wall 2530 extending perpendicular at one end of the surface 2531 to the longitudinal axis 2521 of the implant, FIG. 115, and a wall 2534 opposite to and spaced from the perpendicular wall inclined to the axis 2521. The root between the perpendicular and inclined walls is curved forming a radius. The perpendicular wall 2530 and the inclined wall 2534 define angle 2538a or preferably about 60.degree. therebetween.

The bottom surface 2512b is shown in greater detail in FIG. 117. The ridges on the top and bottom surfaces are the same. In FIG. 117, the planar ridge 2510 has the inclined wall 2534 and perpendicular wall 2530 at opposing ends. Angle 2538b is the same as angle 2538a, about 60.degree., and is between the inclined wall 2534 and the perpendicular wall 2530.

Surgical Technique

The surgical procedures using the devices described herein, relate to discectomy, distraction, endplate preparation, and initial insertion of an implant, such as a spacer or graft include the following steps. Steps 1-7 are common steps to all procedures. Steps 8-10 describe surgical techniques which may differ for each implant insertion and final seating of the implant(s).

Step 1: FIGS. 118 and 119 depict a representative pedicle screw system 2600 and includes inserting the pedicle screws into the spine. Pedicle screws 2606a and 2606b of the pedicle screw system 2600 are representative of the screws used in the selected technique by the surgeon. FIG. 118 shows a portion of a spinal column 2602. The pedicle screw system 2600 is shown attached to the spinal column 2602.

Step 2: The facet area 2610 is removed using chisels shown in FIGS. 26-33. The facet area 2610 is removed to gain access to disc space on one side of the spine only. After removing the facet joint 2604, access to the disc is provided and shown by darkened area 2612, in FIG. 120.

In FIG. 120, the vertebrae including the disc space 2618 are accessed through a single opening 2612 on one side of the annulus 2614, FIG. 121. The significant peripheral regions of the spine, FIG. 121, are labeled: anterior, posterior, medial, and lateral for reference, and a lamina 2608 is also shown.

Step 3: This step includes distracting the vertebrae 2622a and 2622b, for example, FIG. 122, with the lamina spreaders of FIGS. 79-84, represented by the spreader 2640a schematically representative of the spreader and which may be an offset spreader. The distraction also may be made by pedicle screw distractors. The spreader 2640a, of the offset type, spans an area 2642 in which the lamina has been removed. The pedicle screw system is from whatever system the surgeon has selected.

During the course of the procedure and specifically the “trialing” stage, the surgeon may elect to increase the amount of distraction using lamina spreaders or distractors to ascertain the most appropriate sized implant. Lamina spreaders or distractors are represented by instruments 2640a, 2640b, and 2640c shown in FIGS. 122, 123. A prior art lamina spreader is shown in FIGS. 79-81, which may be used as shown in FIGS. 122 and 123.

The offset lamina spreader 1600 according to an embodiment of the present invention, shown in FIGS. 82-84, may be used as representative spreader 2640a where the lamina has been previously removed as in area 2642. When using offset lamina spreader 1600, the offset gap between the jaws approximates the gap along the length of the spine (area 2642) where the removed lamina would have originally been.

Typically, each pedicle screw system has a pedicle screw distractor which the surgeon may choose. Distracting the vertebrae increases the height of the disc space using the lamina spreaders, or the distraction of a pedicle screw construct, or any means the surgeon deems appropriate.

For example, referring to FIG. 122, instrument 2640a represents an offset lamina spreader. The lamina has been removed from area 2642. The offset lamina spreader 2640a legs 2641a and 2641b span the space 2642 created by the removed lamina. Instrument 2640b is representative of a generic pedicle screw distractor which typically may include scissors, parallel action scissors, or other similar type instruments. The pedicle screw distractor 2640b, FIG. 122, includes legs 2645a and 2645b which span the distance between the screws 2606a and 2606b. Instrument 2640c is representative of lamina spreaders which may include a scissors-type spreader with a bend at the jaws to substantially remove the handles from the surgeon's field of view.

Step 4: This involves removing a posterior lip of vertebral bodies using chisels, if necessary, such as the prior art chisels shown in FIGS. 26-29, or the chisels according to an embodiment of the present invention shown in FIGS. 30-33. Removing the posterior lip facilitates the insertion of the trials and implant, and also will allow better assessment of disc height using the trials. A representative chisel 2648 is shown in FIGS. 124 and 125. The border 2650 (shown by the darkened border) of a disc space 2650a is shown in FIG. 125. A posterior lip area 2652 is shown in FIG. 125 with the chisel 2648 positioned adjacent to the posterior lip area 2652.

Step 5: The disc material is removed using rongeurs, serrated cup curettes and loop curettes. The rongeurs, prior art, are shown in FIGS. 73-78. Serrated cup curettes according to an embodiment of the present invention include straight serrated cup curettes 800, FIGS. 34-40, inclined right serrated cup curettes 900, FIGS. 44-50, and inclined left serrated cup curettes 1000, FIGS. 51-57. In FIGS. 58-62, prior art loop curettes are shown and in FIGS. 63-72, loop curettes 1200 and 1300 according to an embodiment of the present invention are shown.

In FIGS. 126 and 127, the technique of step 5 includes removing disc material in a disc space 2651 comprising a nucleus and inner annulus 2652 represented by the dotted area in FIGS. 126 and 127. As much of the outer annulus 2654, shown by the shaded area around the perimeter of the disc space 2654, is left intact. The disc material is removed using prior art rongeurs shown in FIGS. 73 and 76. Also, serrated cup or loop curettes may be used, such as for example, straight serrated cup curette 800, FIGS. 34 and 35, serrated cup curette 900 inclined right, FIGS. 44 and 45, and/or serrated cup curette 1000 inclined left, FIGS. 51 and 52. A straight loop curette may be used, FIGS. 58 and 59, and/or a loop curette 1200 inclined left and down, FIGS. 63-67, and/or a loop curette 1300 inclined right and down, FIGS. 68-71. Any of the curettes above may be used to remove the disc material 2652. The curettes that have bent shaft portions with the curettes are bent (inclined left and down or inclined right and down) to facilitate removal of disc material in the portion of the disc space furthest from the access opening to the disc space (contralateral side). The contralateral side 2656 of the disc space 2651, FIG. 126, is opposite the disc space opening 2660. Surgical instruments 2658a and 2658b are shown in FIGS. 126 and 127 which are representative of the configuration of the curettes (and also the rongeurs, not shown) described above. Instrument 2658a is straight, and instrument 2658b is bent and shown accessing the contralateral side 2656 of the disc space 2651 relative to opening 2660.

To prepare the endplates of adjacent vertebrae for fusion i.e., create bleeding bone, the surgeon may use rasps 1700 and 1800, FIGS. 85-93, and serrated cup and looped curettes described above. The surgeon may use the straight cup curettes 800, FIGS. 34-40, the serrated cup curette 900 inclined right, FIGS. 44-50, the serrated cup curette 1000 inclined left, FIGS. 51-57, a looped curette 1200 inclined left and down, FIGS. 63-67, and/or a looped curette 1300 inclined right and down, FIGS. 68-72, to obtain a bed of bleeding bone.

The bleeding bone 2682 is indicated by the hatched area within the disc space 2688 shown in FIGS. 128 and 129. The outer annulus 2688a extends along the perimeter of the disc space 2688 and is depicted by the darkened area in FIGS. 128, 129. The outer annulus 2688a is absent where an opening 2686 to the disc space is present. Representative instruments 2684a and 2684b are shown in FIGS. 128 and 129. The instrument 2684a may be, for example, the straight rasp 1700, FIGS. 85 and 86. The instrument 2684b may also be, for example, the inclined rasp 1800, FIGS. 87 and 88. The instrument 2684a, FIG. 128, is at the entrance to the disc space 2688 at the opening 2686. The instrument 2684b has traversed the disc space 2688, FIG. 129, and is in the disc space contralateral side 2690.

Step 6: The final discectomy and endplate preparation is completed using the serrated cup curettes 800 and 1000, FIGS. 34, 35, 51 and 52, or the loop curettes 1200, FIGS. 63-72, and/or rasps 1700, FIGS. 85-93.

Step 6 includes assessing the disc space for a proper size implant using lordotic trials 2100 shown in FIGS. 105-108, for both lordotic posterior implants, such as implant 100, FIG. 1 and non-lordotic posterior block implants, such as implant 200, FIG. 6. The size of the tip of the lordotic implant trial used corresponds to the overall height, width, and maximum length of the lordotic implant 100. The size of the tip of the lordotic implant trial used should also correspond to the overall width and maximum length of the non-lordotic posterior block implant 200, but not necessarily the height of the non-lordotic posterior block implant because the trial is lordotic. Also, removal of a trial can be aided by the use of a slap hammer such as slap hammer 2200, FIG. 110, whose threaded stud 2230 mates with the threaded bore in the trial instrument handle 2150, FIG. 107. The non-lordotic posterior block implants 200, FIGS. 6-10, have parallel sides that engage the vertebrae.

Surgical instrument 2700, FIG. 130, is representative of lordotic implant trials such as the trial 2100, FIG. 105. It is assumed that the height on the contralateral side 2702a of the disc space 2702 is equal to the height of the disc space where the trial head 2700a of the trial 2700 is located. The bleeding bone 2704 is indicated by the hatched area within the disc space 2702 of the vertebral disc 2710, FIG. 130.

The disc space is then assessed for proper size of the implant. Instrument 2800, FIGS. 131 and 132, is representative of inclined humeral spacer trials, such as, for example, inclined humeral spacer trial 2100, FIG. 105. The thickness size of the head 2802 of the representative trial 2800 corresponds to the overall height of the non-lordotic implants, i.e., the vertebral disc space height. The diameter of the representative trial 2800 corresponds to the maximum overall width, top to bottom of the figure, FIG. 113, of the humeral TLIF spacer 2500, FIGS. 113-117, the lordotic implants 100, FIG. 1, and non-lordotic block implants 200, FIG. 6, have a smaller transverse width than the humeral spacer. The trial head 2802 is cylindrical and does not necessarily correspond to the shape of the implant. The trial head 2802 shape is made universal for the various TLIF implants reducing the number of instruments required for the procedure. Removal of the trial can be aided by the use of a slap hammer, such as slap hammer 2200, FIG. 110, which threads into the back of the handle portion of the trial. These trials have angled shaft portions at the trial head and are intended to be used with feeler gauges to approximate the height of the disc space within the area that was prepared for implant insertion through the endplate preparation. The trial is moved about in the disc space.

In FIGS. 131 and 132, the trial 2800, with a bent shaft portion, can be flipped over, i.e., rotated 180.degree., to assess both sides of the disc space 2804. The instrument 2800, FIG. 131, enters the disc space 2804 through opening 2810 where the outer annulus 2814 (shown by the darkened area) around the perimeter of the disc space 2804 is removed. The instrument 2800, FIG. 131, is withdrawn from the disc space, and is flipped, i.e., rotated 180.degree., FIG. 132. The rotated instrument 2800 is then inserted into the disc space 2804 through opening 2810, flipped, i.e., rotated 180.degree., from the orientation of FIG. 131, such that the head 2802 is now positioned at the contralateral side 2808 of the disc space 2804.

In the alternative, lordotic trials may be used which do not have a bent shaft portion at the trial head (not shown). These trials can not be shifted to the contralateral side of the disc space and therefore are not as preferred as the trials with bent angled shaft head portions.

Step 7: The surgeon then selects an appropriately sized implant based on Step 6 above, as well as, any pre-operation planning. The size of the graft/implant should be sufficient to restore disc space, however, the implant should be inserted and subsequently shifted into its final position under minimal resistance. If necessary, distraction of the disc space may be increased to accomplish this.

In a case where straight lordotic trials are used by the surgeon, the surgeon cannot shift the trial to the contralateral side of the disc space. In this case, the surgeon inserts the straight trial into the disc space through a single opening and assumes that the height of the disc space will be uniform through a medial/lateral width of the prepared disc space. The trial 2100, FIGS. 105-109, has an inclined shaft head portion, and thus can be shifted inside the disc space to assess the height throughout the prepared disc space including the contralateral side of the disc space.

Step 8: The lordotic posterior ramp implant 100 is attached, or the non-lordotic posterior block implant 200, represented by implant 3002, FIGS. 133, 134, to a posterior ramp insertion instrument 3000, FIGS. 133, 134, such as instrument 2300, FIG. 111. The implant is then impacted into the disc space through the disc space opening using the insertion instrument. The insertion instrument 3000 is removed once the implant 3002 is in the disc space. Supplemental bone grafting material 3020 is shown by the darkened area, FIGS. 133 and 134. The bone grafting material 3020 may be packed as specified by the surgeon, into the disc space 3014 prior to inserting the first implant such as implant 3002, FIG. 133. Additionally, this bone grafting material 3020 may be packed in the anterior portion of the disc space 3014 or into any other portion of the disc space elected by the surgeon.

FIGS. 133 and 134 show two options for the final seating of the implant 3002a. In FIG. 133, the representative instrument 3000 is inserted through opening 3010 holding implant 3002. The implant 3002 can be turned into the desired position by the surgeon. In FIG. 134, the representative instrument 3000 has the graft 3002 positioned toward the contralateral side 3018 of the disc space 3014, and the graft 3002 is reversed in relation to it's orientation in FIG. 133. Because the implants will be shifted after initial placement, it is important to maintain distraction and choose an implant size that will be easily shifted within the disc space to its final seating after insertion into the disc space.

The surgical instrument 3100 shown in FIGS. 135 and 136 is representative of all bone tamps, for example, the “L” shaped bone tamp 1900, FIGS. 94, 95, and the inclined tamp 2000, FIGS. 99, 100. The implant 3002, FIGS. 135, 136, is moved to different positions by the instrument 3100. Supplementary bone grafting material 3020 is depicted by the darkened areas. Bleeding bone 3016 is indicated by the hatched area within the disc space 3014.

The surgeon may use an “L” bone tamp 1900 shown in FIGS. 94 and 95 or an inclined tamp 2000 shown in FIGS. 99 and 100 as desired. The tamps may be used to shift the implant 3002 to it's position of “final seating”. The implant 3002 final seating position may be the contralateral side 3018 of the disc space 3014 shown in FIG. 135, or along the anterior portion 3018a of the disc space 3014 shown in FIG. 136 or in any other position as set by the surgeon.

FIGS. 137, 138 and 139 show three options for the final seating of a first implant 3002a and a second implant 3002b according to the procedure disclosed in step 7 above. Supplemental bone grafting material 3020 is shown in different locations in FIGS. 137-139 as a darkened area. According to step 7 of the procedure, the first implant 3002a is shifted into its “final seating” position on the contralateral side, FIG. 135, or anterior side, FIG. 136, then any supplementary bone grafting material 3020 is packed around the implant 3002a, if desired. The grafting material is packed prior to placing a second implant 3002b, FIGS. 137-139. The first and second implants 3002a, 3002b are shown in the disc space 3014 in various positions. The final position of the second implant 3002b may be altered using a representative bone tamp 3200 shown in FIG. 137, and for example, the bone tamp 2000, FIGS. 99, 100.

The first and second implants 3002a, 3002b can be facing one another as positioned in FIG. 137. Also, the first and second implants 3002a, 3002b can be placed adjacent to each other along a posterior-anterior plane. In FIG. 139, the surgeon can also implant the first and second implants 3002a, 3002b such that their orientation with respect to each other is similar to an “I” beam. With the surgical option shown in FIG. 138, the anterior most implant 3002a may be larger in height than the more posterior implant 3002b, so that an overall lordosis is imparted to the disc space. Alternatively, also in the case shown in FIG. 138, to better conform to the natural concavity of the vertebral endplates, the anterior most implant 3002a may be smaller in height and the more posterior implant 3002b may be taller. The steps above are common to all surgical techniques.

Step 8: This step includes attaching an implant, such as for example, a humeral spacer or posterior implant to either the posterior insertion instrument 2300, FIG. 111, or the tweezer insertion instrument 2400 shown in FIGS. 112 and 112A. The spacer or implant can then be impacted into the disc space opening.

A surgical instrument 3200, FIG. 140, is representative of for example, the tweezer insertion instrument 2400, FIGS. 112 and 112a. The instrument 3200 grasps the implant 3202, FIG. 140, such as the humeral spacer. The humeral spacer 3202, FIGS. 140-143, has a substantially central opening 3208 with supplemental bone grafting material 3220 inside the opening 3208. The posterior ramp insertion instrument 2300, FIG. 111, may also be used to place the implant 3204 into position in the disc space 3210. Supplemental bone grafting material 3220 is shown by the darkened area in FIGS. 140-143. Bleeding bone 3216 is indicated by the hatched area within the disc space 3210.

Once the implant 3204 is in the disc space 3210, the instrument 3200 is removed by the surgeon. Supplemental bone grafting material 3220 may be packed into the disc space 3210 prior to inserting the implant 3204 or multiple implants, as well as, into the opening 3208 of the humeral spacer implant 3220 if the surgeon chooses, FIGS. 140-143.

A bone tamp instrument 3250, FIG. 141, is representative of bone tamps that may be used. The bone tamp 3250 is used to shift the humeral spacer 3204. An “L” shaped bone tamp 1900 is shown in FIGS. 94 and 95, and an inclined bone tamp 2000 is shown in FIGS. 99 and 100. Both tamps 1900 and 2000 can be used to shift the humeral spacer 3220 into the position shown in FIG. 141. In FIG. 141, the humeral spacer 3202 is shifted so that its length is aligned substantially parallel with a plane along a medial/lateral axis 3252, and thus the humeral spacer rests at the anterior portion of the disc space 3210, FIG. 141.

Alternatively, as shown in FIGS. 140, 142, the humeral spacer 3204 may be left in its initial diagonal position with bone grafting material 3220 on both sides of the implant 3204, as described in step 8. In a further alternative, the humeral spacer 3204 may be positioned on the anterior/posterior axis 3254, FIG. 143. After steps 1-7, the surgical techniques may differ for implant insertion and final seating of the implant(s) at the discretion of the surgeon. Steps 8-10 are employed by the surgeon as deemed necessary since they are not common to all the surgical techniques.

This step also includes positioning supplementary bone grafting material 3220 (shown as the darkened areas in FIGS. 142 and 143) around the implant 3204, after the implant 3204 has been shifted into its final position, as shown in FIGS. 142 and 143.

As discussed above, after the surgeon has selected the appropriately sized implant, the surgeon holds the implant using an insertion instrument. Preferably, the implants for the TLIF systems are shifted after their initial placement. It is therefore important, after the implant is inserted, to maintain distraction and to choose an implant that will shift within the disc space to its final seating position.

If a surgeon chooses to use a posterior ramp implant, the insertion instrument that should be used is the instrument 2300, FIG. 111. The surgeon may choose to insert one or more posterior ramp implants into the disc space at various angles and at a determined depth, after which, the surgeon unscrews the insertion instrument from the ramp implant. The surgeon may elect to adjust the location of the implant to a “final seating” position using any of the bone tamps, shown in FIGS. 94-104, such as bone tamps 1900 and 2000. Some typical locations are shown in FIGS. 133-139.

If the surgeon chooses to use a non-lordotic posterior block implant, such as implant 200, the surgeon will use the same procedure as step 7, FIGS. 133-139.

If the surgeon chooses to use the humeral spacer, the spacer may be secured to the insertion instrument, depending on the interface.

a. When using a ramp implant such as implant 100 threaded hole interface, the prior art insertion instrument 2300, FIG. 111, will be used. The surgeon can insert the implant into the disc space, unscrew the inserter, and use any of the bone tamps 1900 or 2000, FIG. 94 or 99, respectively, to adjust the implant into it's final seating position. The final seating may include shifting the implant to the anterior portion of a disc space endplate which is in-line with the overall medial/lateral width of the disc space, or, alternatively, shifting the implant to be along the length of the disc space which is in-line with the anterior/posterior plane of the disc space.

b. The tweezer insertion instrument 2400, FIGS. 112-112C, is used to grasp the dovetail undercut interface of an implant. The tweezer insertion instrument 2400 is used similarly to the prior art anterior ramp insertion instrument 2300, shown in FIG. 111. The tips of the tweezer insertion instrument 2400, FIGS. 112b-112c, engage the dovetail depressions or recesses of the implant as the handles of the inserter are squeezed together, as described above in reference to FIGS. 112a-112c. Gentle impaction may be applied to the back end of inserter 2400 to place the implant into its initial seating, such as diagonally within the disc space. When the knob 2466, FIG. 112a, is unscrewed, the springs 2416a, 2416b between the handles 2410a, 2410b urge the handles apart and, therefore, the jaws 2430a, 2430b disengage the implant 2450. The tamps 1900 and 2000, FIGS. 94 and 99, position the implant such as the humeral spacer into its “final seating” position. Additionally, bone graft material 3220, FIG. 140, can be packed within the opening 3208 in the humeral spacer 3204. Thereafter, the final seating procedure described above should be used.

Step 9: This includes packing additional bone grafting material within the disc space, after the implant(s) is/are in the “final position”, as described in Step 7.

Step 10: This includes the removal of the distraction instrument, such as lamina spreaders or pedicle screw distractors.

During steps 9 and 10, the surgeon finishes the placing of the pedicle screw system 2000, FIGS. 118 and 119. The placing of the pedicle screw system is completed in a manner consistent with the placement specifications used for the particular pedicle screw system. The pedicle screw system is used to ensure that stability has been restored to the affected disc space 3300 of the spine, FIG. 144.

Step 11: This includes applying compression via the pedicle screw system 2600, FIGS. 118, 144, to help provide lordosis to the disc space 3300. This includes compressing the pedicle screw system 2600 downwardly and locking the pedicle screw system in place. The locked pedicle screw system provides stability to the vertebrae levels of the spine being fused by the implant(s). In addition, the cantilever action of the compression of the pedicle screw system against the implant(s) 3300 allows the surgeon to provide some lordosis to the vertebrae level(s) being fused.

It will occur to one of ordinary skill that modifications may be made to the various embodiments disclosed herein. The instruments have been shown by way of example, and other instruments, not shown may also be used. For example, distractors, trials, insertion instruments, chisels and rasps may take a wide variety of shapes and configurations as known in this art. It is intended that the invention be defined by the appended claims. Where specific descriptions are given, these are given by way of example, and not limitation. There is no intent herein to define the specific terms to any particular definition that is given by way of example and that the ordinary and customary meaning as would interpreted by one of ordinary skill applies to such terms. Exemplary embodiments are shown and are intended to be representative of other alternative embodiments not shown.

What is claimed is:

1. A spinal bone implant comprising: a body made of bone, the body defining a longitudinal axis, and having opposing end walls transverse to the longitudinal axis, substantially parallel top and bottom surfaces, and opposing side walls in communication with the top and bottom surfaces and end walls, one of the side walls having a concave portion and the other side wall having a convex portion in opposing relation to the concave portion, the top and bottom surfaces for bearing against respective adjacent vertebrae defining a disc space therebetween; and spaced parallel ridges on the top and bottom surfaces transverse to the longitudinal axis; the body having a bore in one of the end surfaces and side walls in communication with a body outer peripheral surface extending inwardly towards a central region of the body.

2. A spinal bone implant comprising: a body made of bone; the body having top and bottom surfaces and opposing side walls transverse to the top and bottom surfaces; the body having an opening therethrough defining an axis and in communication with said top and bottom surfaces, the opening being defined by a radius from the axis, the top and bottom surfaces for bearing against respective adjacent vertebrae in a disc space therebetween, at least one of the top and bottom surfaces including a plurality of spaced concentric ridges; the body at least one outer peripheral side wall having a bore extending transverse to and radially inwardly towards the axis of the opening.

3. The implant of claim 2 wherein both the top and bottom surfaces of the body include said plurality of spaced concentric ridges.

4. The implant of claim 3 wherein the plurality of spaced concentric ridges on the top and bottom surfaces lie in parallel planes.

5. The implant of claim 2 wherein the spaced concentric ridges overlie substantially all of the at least one of the top or bottom surfaces.

6. The implant of claim 2 wherein each of the plurality of spaced concentric ridges has two inclined side walls defining an angular relation to each other and converging at a crest edge, each of the side walls of each of the ridges has substantially the same angular orientation to a reference plane.

7. The implant of claim 2 wherein the plurality of spaced concentric ridges have two inclined side walls converging at an edge, the edges of the plurality of spaced concentric ridges lying in a plane, the inclined side walls being substantially at the same angle to the plane.

8. A spinal bone implant comprising: a body made of bone; the body having top and bottom surfaces defining top and bottom planes, respectively, and opposing side walls, the body defining a substantially central opening in communication with the top and bottom surfaces and extending transverse to the planes, the opening defining a central longitudinal axis and having a radius from the central longitudinal axis, the top and bottom surfaces for bearing against respective adjacent vertebrae, the top and bottom surfaces each including a plurality of spaced parallel linear ridges, the ridges on the top surface having a first angular orientation relative to the longitudinal axis, the ridges on the bottom surface having a second angular orientation relative to the longitudinal axis different than the orientation of the ridges on the bottom surface.

9. The implant of claim 8 wherein the body defines a bore in communication with an outer peripheral surface of the body and extending inwardly towards the central longitudinal axis.

10. The implant of claim 9 wherein the ridges on the top and bottom surfaces are offset at about 90.degree. from each other.

11. The implant of claim 10 wherein the plurality of spaced ridges have two inclined side walls converging at an edge, the edges of the plurality of spaced ridges define a plane, the inclined side walls being at about a 60.degree. to the plane.

12. The implant of claim 10 wherein the plurality of spaced ridges each have first and second side walls converging at an edge, the edges defining a first plane, the ridges having the first side wall at about a 90.degree. to the first plane, the second side wall being about 60.degree. to the first plane.

13. The implant of claim 12 wherein at least one of the side walls defines at least two notches in spaced relation to each other, each notch having two internal side walls in angular relation to each other.

14. The implant of claim 13 wherein the internal side walls of each of the notches are at an acute angle to each other.

15. The implant of claim 13 wherein the two notches are coplanar.

16. The implant of claim 13 wherein the notches are triangular in cross section.

17. The implant of claim 10 wherein the implant has a perimeter that has a planar section contiguous with an arcuate section.

18. A chisel for preparing adjacent vertebrae for insertion of a spinal implant into the disc space defined by the vertebrae comprising: a shaft having a central longitudinal axis and distal and proximal ends, the shaft having a shaft head portion having a second longitudinal axis inclined relative to the shaft longitudinal axis and located at the shaft distal end; a handle coupled to the shaft at the shaft proximal end; and a cutting head coupled to the shaft head portion extending distally from the shaft head portion, the cutting head having a linear cutting edge extending inclined to the shaft longitudinal axis and normal to the shaft head portion second axis.

19. The chisel of claim 18 wherein the shaft head portion is at an angle to the longitudinal axis in the range of about 10.degree. to about 60.degree.

20. The chisel of claim 18 wherein the cutting head and shaft head portion are coaxial and are at an angle to the longitudinal axis of about 10.degree. to about 20.degree.

21. A curette for preparing adjacent vertebrae for insertion of a spinal implant into a disc space defined by the vertebrae comprising: a shaft defining a longitudinal axis and having distal and proximal ends; a handle coupled to the shaft at the shaft proximal end; a curette head coupled to the shaft at the shaft distal end, the curette head having a cutting surface that is oriented inclined to the longitudinal axis for scraping vertebral material, the cutting surface defining a perimeter of the curette head, the surface including spaced serrations.

22. The curette of claim 21 wherein the serrations include a plurality of teeth separated by notches, the teeth being defined by linear cutting edges.

23. The curette of claim 22 wherein the notches are in the form of slits normal to the cutting edges.

24. The curette of claim 22 wherein the teeth define coplanar cutting edges.

25. The curette of claim 22 wherein the shaft includes a distal shaft head portion terminating at the curette head, the distal shaft head portion extending inclined to the shaft longitudinal axis.

26. The curette of claim 25 wherein the distal shaft head portion is inclined in the range of about 50.degree. to 60.degree. to the longitudinal axis.

27. A curette for preparing adjacent vertebrae for insertion of a spinal implant into the disc space defined by the vertebrae comprising: a shaft having a longitudinal axis and distal and proximal ends; a handle coupled to the shaft at the shaft proximal end; a shaft head portion coupled to the distal end of the shaft and terminating with a curette head, the distal shaft head portion being inclined to the longitudinal axis in first and second different planes, the first plane being defined by the longitudinal axis, the second plane being normal to the first plane.

28. The curette of claim 28 wherein the shaft head portion is inclined to the longitudinal axis in the first plane at an angle of about 50.degree. to about 60.degree., the curette head lying in the first plane.

29. The curette of claim 28 wherein the shaft head portion and the curette head are each inclined to the longitudinal axis in the second plane at an angle of about 10.degree. to about 30.degree.

30. The curette of claim 28 wherein the curette head is a loop curette.

31. A lamina spreader for separating adjacent vertebrae for insertion and manipulation of a spinal implant in a disc space defined by the vertebrae comprising: an upper arm and a lower arm pivotably connected, both the upper and lower arms each having a handle portion at a proximal end in spaced relation to each other and resiliently biased to oppose one, both the upper and lower arms each having a jaw at a distal end in spaced relation to each other, both the upper and lower jaws terminating with a tip; and a locking device adjustably attached to the upper and lower handle portions and having a locking member for holding the handle portions in spaced relation against the bias to oppose one another.

32. The lamina spreader of claim 31 wherein the upper and lower jaw portions are substantially parallel.

33. The lamina spreader of claim 31 wherein the upper and lower jaw portions define a space therebetween in a quiescent position.

34. The lamina spreader of claim 31 wherein the upper and lower jaw portions define a space therebetween between of about 21 to 25 millimeters.

35. A rasp for preparing vertebrae and a disc space between adjacent vertebrae during a spinal implant surgical procedure comprising: a shaft having a central longitudinal axis and distal and proximal ends; a handle coupled to the shaft at a proximal end of the shaft; and a rasp head coupled to the shaft at the distal end of the shaft where the rasp head has top and bottom surfaces and a side wall, at least one of the top and bottom surfaces includes a plurality of spaced teeth extending angularly from the top or bottom surface toward the proximal end of the shaft.

36. The rasp of claim 35 wherein the teeth include a first side wall perpendicular to the top surface and a second side wall at about a sixty degree angle from the first side wall and the first and second side walls converge at the crest of the teeth.

37. The rasp of claim 36 wherein the apex is about 0.04 mm from the top or bottom surface at the roots of the teeth.

38. The rasp of claim 36 wherein the shaft includes a distal shaft head portion terminating in the rasp head, the distal shaft head portion being inclined along a first plane defined by and in relation to the longitudinal axis.

39. The rasp of claim 38 wherein the distal shaft head portion is inclined about 55.degree.

40. The rasp of claim 38 wherein the distal shaft head portion is inclined between 50 and 60.degree.

41. A tamp for manipulating and seating a spinal implant inserted into a disc space defined by two adjacent vertebrae, comprising: a shaft having a longitudinal axis and distal and proximal ends; a handle coupled to the shaft at the shaft proximal end, the handle having an impact receiving surface at its proximal end; a tamp head coupled to the distal end of the shaft, the tamp head having a planar distal end wall transverse to the longitudinal axis, the end wall forming an impact surface, a first side wall inclined to the longitudinal axis and a second side wall parallel to the longitudinal axis, the inclined wall being intermediate the second side wall and the shaft.

42. The tamp of claim 41 wherein the end wall and second side wall are planar and roughened with an implant gripping surface.

43. The tamp of claim 42 wherein the roughened gripping surface comprises teeth in a two dimensional array.

44. The tamp of claim 41 wherein the inclined side wall is contiguous with the shaft and the first side wall, the end wall and first side wall being normal to each other.

45. The tamp of claim 44 wherein the inclined side wall is at an angle of about 45.degree. to the shaft longitudinal axis.

46. The tamp of claim 41 wherein at least a portion of the end wall and side wall each include diamond shaped knurling.

47. The tamp of claim 41 wherein the shaft has a proximal portion and a distal portion, the distal portion being inclined relative to the proximal portion and terminating in the tamp head.

48. A trial for measuring in a disc space defined by two adjacent vertebrae the space between adjacent vertebrae to size a spinal implant, comprising: a shaft having a longitudinal axis and distal and proximal portions; a handle coupled to the shaft proximal portion; and a trial head coupled to the distal portion; the shaft distal portion being inclined relative to the shaft proximal portion.

49. The trial of claim 48 wherein the angle of the distal portion to the proximal shaft portion is between about 50.degree. to about 60.degree.

50. An implant insertion instrument for inserting a spinal implant into a disc space defined by adjacent vertebrae comprising: a first arm and a second arm each having proximal and distal ends and a longitudinal axis, the arms being pivotally inter-connected in a scissor-like arrangement; opposing handles in spaced relation to each other secured to the proximal end of the arms; a bias member for resiliently biasing the handles apart; a jaw at the distal end of each arm and opposing one another, the arms being arranged such that pivotally displacing the handle portions toward one another displaces the opposing jaws toward one another; each jaw terminating in an implant gripping tip, the tips each having a planar end wall normal to the longitudinal axis of the corresponding arm and a side wall inclined to the longitudinal axis in opposing mirror image relation to each other; and a locking device for locking the handle portions in spaced relation against the bias.

51. A method of inserting an implant into a disc space defined by adjacent vertebrae of a spine, comprising: inserting a first implant into the disc space through an opening in the perimeter of the disc space at the lateral side of the perimeter to a lateral side of the disc space in a transforaminal region of the disc space; and displacing the inserted first implant to a contralateral side of the disc space into a first orientation.

52. The method of claim 51 including rotating the implant after it is inserted to a second orientation during or prior to displacing the implant to the first orientation.

53. The method of claim 51 including inserting a second implant into said disc space through said opening and orienting said second implant in a predetermined orientation relative to the first orientation of the first implant.

54. The method of claim 53 wherein the first and second implants have the same configuration.

55. The method of claim 53 wherein the first and second implants each have a C-shape with a concave side wall, the concave side walls facing in the same direction.

56. The method of claim 53 wherein the first and second implants each have a C-shape with a concave side wall, the concave side walls facing in an opposite direction to each other.

57. The method of claim 53 wherein the disc space has an anterior-posterior axis, the first and second implants each have a C-shape with a concave side wall, the concave side walls facing in the same direction parallel to the anterior-posterior axis.

58. The method of claim 53 wherein the disc space has an anterior-posterior axis, the first and second implants each have a C-shape with a concave side wall, the concave side walls facing in an opposite direction to each other and parallel to the anterior-posterior axis.

59. The method of claim 53 wherein the disc space has an anterior-posterior axis, the first and second implants each have a C-shape with a concave side wall, the concave side walls facing in the a direction normal to the anterior-posterior axis.

60. The method of claim 53 wherein the disc space has an anterior-posterior axis, the first and second implants each have a C-shape with a concave side wall, the concave side walls facing in a direction toward each other and inclined relative to the anterior-posterior axis.

61. The method of claim 53 wherein the disc space has an anterior-posterior axis, the first and second implants each have a C-shape with a concave side wall, the concave side walls facing in an opposite direction to each other and normal to the anterior-posterior axis.

62. The method of claim 53 wherein the disc space has an anterior-posterior axis, the first and second implants each have a C-shape with a concave side wall, the concave side walls facing in a direction inclined to the anterior-posterior axis on the respective lateral and contralateral sides of the disc space.

63. A method of preparing intervertebral disc space for receiving at least one implant comprising the steps of: a) distracting the disc space; b) forming an opening in the perimeter of the disc space on a lateral or contralateral side of the disc space; c) preparing the disc space on the lateral and contralateral sides of the space through the opening using at least one of a cup curette and a loop curette including a curette with a shaft head portion inclined to the longitudinal axis of the shaft; d) inserting a rasp including a rasp with a shaft head portion inclined to the longitudinal axis of the shaft through the opening to further prepare the disc space first on one of and then on the other of the lateral and contralateral sides; e) measuring the size of the first and second of the sides with a trial including a trial with a shaft head portion inclined to the longitudinal axis of the trial shaft inserted through the opening; f) repeating steps d-e until the disc space matches the size of an implant for insertion into that disc space; and then h) inserting the at least one matched implant into the disc space.

64. The method of claim 63 including manipulating the at least one implant in the disc space to a final implant position by rotation and/or other displacement.

65. The method of claim 63 including orienting the at least one implant inclined to the anterior-posterior axis of the spine.

66. The method of claim 65 including facing a first side of the implant in either of two opposite directions transverse to the anterior-posterior axis.

67. The method of claim 65 including positioning the implant at an angle such that the at least one implant is along a posterior side of the disc space.

68. The method of claim 63 further including inserting a plurality of implants of substantially the same configuration and having corresponding first sides, the first sides facing in the same direction in the disc space.

69. The method of claim 63 further including inserting a plurality of implants of substantially the same configuration and having respective corresponding first sides, the manipulation for manipulating the first sides facing each other.

70. The method of claim 63 wherein the at least one implant comprises a plurality of implants of substantially the same configuration and having corresponding first sides, the manipulation for manipulating the first sides facing in opposite directions.

71. The method of claim 70 wherein the manipulation includes rotating or displacing the at least one implant with an L-shaped impact tool and impacting the implant into the final implant position.

72. The method of claim 70 wherein the manipulation includes positioning the at least one implant in the intervertebral disc space along an anterior wall of the disc space substantially perpendicular to the anterior-posterior axis.

73. The method of claim 70 wherein the manipulation includes positioning the at least one implant in the intervertebral disc space diagonally across the disc space.

74. The method of claim 63 wherein the manipulation includes positioning the at least one implant in the intervertebral disc space along a posterior-anterior axis.

75. A spinal implant comprising: a bone having opposing top and bottom surfaces for engaging and gripping adjacent vertebrae in a spinal disc space; and a plurality of parallel arrays of linear teeth on each said surfaces for engaging and gripping the vertebrae, the arrays on one surface extending in a first direction and the arrays on the other surface extending in a second direction, the first direction being transverse to the second direction.

76. The implant of claim 75 wherein the first direction is orthogonal to the second direction.

77. The implant of claim 75 wherein the implant has a perimeter defined by side walls terminating at said top and bottom surfaces, said side walls being planar and defining planes transverse to said top and bottom surfaces.

78. The implant of claim 77 wherein the implant has six planar side walls and is symmetrical.

79. An implant impact insertion instrument comprising: a handle; an elongated shaft defining a longitudinal axis and connected to the handle at one shaft end and terminating at a second shaft end remote from the handle; the shaft having a shaft portion defining a further longitudinal axis inclined relative to the longitudinal axis and terminating in a free end distal the shaft second end; and an impact head at the free end of the inclined shaft portion, the impact head having an impact end surface and a plurality of side walls terminating at and defining the impact surface, the end surface having a roughened surface for gripping an implant to be inserted, the end surface being inclined relative to said longitudinal axis and extending transverse to the further longitudinal axis.

80. The instrument of claim 79 wherein each of the side walls are planar and normal to said end surface, said end surface being normal to said further longitudinal axis, said end surface comprising a two dimensional array of symmetrical pyramidal teeth.
